SEQUENCE LISTING (5 sequences)

SEQ ID NO: 1 - protein, 195 aa, homo sapiens
MGRARDAILDALENLTAEELKKFKLKLLSVPLREGYGRIPRGALLSMDALDLTDKLVSFYLETYGAELTANVLRDMGLQEMAGQLQAATHQGSGAAPAGIQAPPQSAAKPGLHFIDQHRAALIARVTNVEWLLDALYGKVLTDEQYQAVRAEPTNPSKMRKLFSFTPAWNWTCKDLLLQALRESQSYLVEDLERS

SEQ ID NO: 2 - protein, 90 aa, homo sapiens
MGRARDAILDALENLTAEELKKFKLKLLSVPLREGYGRIPRGALLSMDALDLTDKLVSFYLETYGAELTANVLRDMGLQEMAGQLQAATH

SEQ ID NO: 3 - protein, 89 aa, homo sapiens
AAKPGLHFIDQHRAALIARVTNVEWLLDALYGKVLTDEQYQAVRAEPTNPSKMRKLFSFTPAWNWTCKDLLLQALRESQSYLVEDLERS

SEQ ID NO: 4 - protein, 176 aa, homo sapiens
MGRARDAILDALENLTAEELKKFKLKLLSVPLREGYGRIPRGALLSMDALDLTDKLVSFYLETYGAELTANVLRDMGLQEMAGQLQAATHQGLHFIDQHRAALIARVTNVEWLLDALYGKVLTDEQYQAVRAEPTNPSKMRKLFSFTPAWNWTCKDLLLQALRESQSYLVEDLERS

SEQ ID NO: 5 - protein, 135 aa, homo sapiens
MGRARDAILDALENLTAEELKKFKLQAATHQGSGAAPAGIQAPPQSAAKPGLHFIDQHRAALIARVTNVEWLLDALYGKVLTDEQYQAVRAEPTNPSKMRKLFSFTPAWNWTCKDLLLQALRESQSYLVEDLERS

(19) United States
(12) Patent Application Publication
(10) Pub. No.: US 2021/0079075 A1
(43) Pub. Date: Mar. 18, 2021

(54) NOVEL MEANS AND METHODS FOR TREATING NEURODEGENERATIVE DISEASES

(71) Applicant: Michael Heneka, Bonn (DE)

(72) Inventors: Michael Heneka, Bonn (DE); Eicke Latz, Bonn (DE); Carmen Venegas Maldonado, Bonn (DE); Jochen Walter, Sankt Augustin (DE); Sathish Kumar, Bonn (DE)

(21) Appl. No.: 16/956,255

(22) PCT Filed: Dec. 20, 2018
(86) PCT No.: PCT/EP2018/086441

§ 371 Date: Jun. 19, 2020

(30) Foreign Application Priority Data
Dec. 20, 2017 (EP) PCT/EP2017/083944

(51) Int. Cl. C07K 16/18; C12N 15/113; A61K 31/7105; A61P 25/28

(52) U.S. Cl. CPC C07K 16/18; C12N 15/113; A61K 45/06; A61P 25/28; A61K 31/7105

(57) ABSTRACT

The present invention provides novel means and methods for treating and diagnosing neurodegenerative diseases. In particular, said means and methods include ligands of the apoptosis-associated speck-like protein containing a CARD. Further provided herein are nucleic acids encoding such ligands, and vectors and host cells comprising the same. The present invention further relates to pharmaceutical compositions as well as kits and diagnostic kits.

The present invention relates to compounds, compositions and methods for the treatment of various neurodegenerative diseases, disorders and conditions, particularly those characterized or accompanied by innate immune activation, which may be triggered by the assembly of beta-amyloid peptides (AR) into larger aggregates and plaques. In particular, the present inventors discovered that the inflammasome-dependent recruitment and aggregation of apoptosis associated speck-like protein containing a CARD (ASC) play an important role in Aβ-related pathology.

Genetic and experimental evidence supports a pathogenic role of immune activation in neurodegenerative disorders¹. In Alzheimer's disease (AD), genetic^{2,3} and epigenetic⁴ studies, transcriptome analysis of human AD brains⁵ and expression quantitative trait experiments in monocytes⁶ all support a contributing role of innate immune mechanisms. However, the connection between AD-related immune activation to classical hallmarks of AD is less clear. Assembly of beta-amyloid peptides (Aβ) into pathological seeds and their subsequent aggregation represents one of the key pathologies of AD. A critical role of Aβ for AD manifestation is supported by mutations that lead to increased Aβ production and deposition in familial forms of AD (fAD)⁷. In sporadic AD (sAD), Aβ may play an initiating role and is linked to a complex network of pathological processes, which may converge over time before neurodegeneration prevails and clinical symptoms appear⁸. However, the precise mechanisms underlying Aβ aggregation and spreading of pathology are not fully understood⁹.

Importantly, deposition and spreading of Aβ pathology likely precede the appearance of clinical symptoms by decades¹⁰ and therefore the mechanisms involved in these processes are believed to hold therapeutic potential for AD. Once aggregated, Aβ is sensed by microglial pattern recognition receptors leading to pathological innate immune activation and subsequent production of inflammatory mediators¹¹. Activation of the NACHT, LRR, and PYD domains-containing protein 3 (NLRP3) inflammasome, a central sensor for danger signals, has recently been documented in the brains of AD patients and APP/PS1 transgenic mice¹². Genetic deficiency of NLRP3 or caspase-1 both protect aged APP/PS1 mice from microglial IL-1β production, Aβ-related pathology and development of cognitive decline¹². Previous findings, demonstrating a very early and focal immune activation of IL-1β-positive microglia in similar murine AD models, prompted the question, whether activation of the NLRP3 inflammasome contributes to the progression and spreading of Aβ pathology. After activation, NLRP3 recruits the adapter protein apoptosis associated speck-like protein containing a CARD (ASC) via pyrin (PYD) domain interactions, which triggers ASC helical fibrillar assembly¹³. ASC fibrils then recruit the effector caspase-1 via CARD interactions leading to autoproteolytic activation and subsequent assembly of ASC fibrils into a large paranuclear ASC ‘speck’¹⁴. In fact, prion-like polymerization is a conserved signalling mechanism in innate immunity and inflammation¹⁵. Indeed, besides causing pro-inflammatory IL-1β cytokine activation and release, NLRP3 inflammasome activity also results in the release of assembled ASC specks, which, once released into the intercellular space, can be taken up by neighbouring myeloid cells to sustain the ongoing immune response^{16,17}. ASC expression increases in APP/PS1 animals with age, but not in wild-type mice.

Alzheimer's Disease (AD) is the most common neurodegenerative disorder of aging and the fourth leading cause of death in industrialized societies, surpassed only by heart disease, stroke and cancer, AD affects 5-11% of the population over the age of 65 and 30% of those over the age of 85. However, like many other neurodegenerative diseases, Alzheimer's Disease is still incurable, and available treatment options are merely palliative. Novel therapies and diagnostic methods are urgently needed to enable early detection and treatment of these diseases.

In view of the above, it is the object of the present invention to overcome the drawbacks of current treatment and diagnostic options and to provide novel means and methods for treating, preventing and detecting neurodegenerative diseases such as AD.

This object is achieved by means of the subject-matter set out below and in the appended claims.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements.

Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%.

### DETAILED DESCRIPTION

The spreading of pathology within and between brain areas represents a hallmark of neurodegenerative diseases. The invention is based, in part, on the discovery that the inflammasome-driven formation of apoptosis-associated speck-like protein containing a CARD (ASC) “specks” contributes to β-amyloid (Aβ) pathology in Alzheimer's Disease (AD) and other neurodegenerative diseases. The present inventors discovered that ASC specks released by microglia rapidly bind to Aβ and increase Aβ oligomer and aggregate formation, acting as an inflammation-driven cross-seed for Aβ pathology. Intrahippocampal ASC speck injection resulted in spreading of Aβ pathology in APP/PS1 mice. In contrast, APP/PS1 brain homogenates failed to induce seeding and spreading of Aβ-pathology in ASC-deficient APP/PS1 mice. Surprisingly, the present inventors found that co-application of an ASC ligand blocked augmented Aβ pathology. These findings indicate that inflammasome activation is connected to seeding and spreading of Aβ pathology in neurodegenerative diseases such as Alzheimer's disease, and supports ASC ligands capable blocking ASC aggregation as viable options for treating and preventing such diseases.

The deposition and spreading of amyloid-β aggregates is a key characteristic of Alzheimer's Disease and is considered to be involved in other neurodegenerative diseases as well. Once aggregated, amyloid-β is sensed by microglial pattern-recognition receptors leading to pathological immune activation and activation of the NLRP3 inflammasome. The NLRP3 inflammasome, a multiprotein complex acting as a central sensor for danger signals, recruits ACS and ultimately triggers its assembly into larger aggregates (“specks”), which are released into the intracellular space. The present inventors discovered that released ASC aggregates (“specks”) bind rapidly to amyloid-β and increase the formation of amyloid-β oligomers and aggregates, thereby acting as an inflammation-driven cross-seed for amyloid-β pathology ultimately resulting in neurodegeneration.

ASC ligands according to the invention preferably act as inhibitors of ASC and reduce or abolish its capability of assembling into larger aggregates (“specks”). ASC ligands that prevent or reduce the formation of such ASC “specks” are preferably capable of preventing or reducing the formation of amyloid-β oligomers, aggregates and plaques. The inventive ASC ligands are therefore envisaged to be useful for preventing and treating neurodegenerative diseases, which are preferably characterized by or associated with the formation of ASC aggregates (“specks”) and/or amyloid-β pathology, in particular the formation and spreading of amyloid-β aggregates.

In a first aspect the present invention features a ligand of apoptosis-associated speck-like protein containing a CARD (ASC) for use in a method of treatment or prevention of neurodegenerative diseases.

The term “ligand” as used herein refers to (macro-)molecules capable of interacting with, preferably binding to, apoptosis-associated speck-like protein containing a CARD (ASC). Preferably, the ligand specifically interacts with, or binds to, ASC. “Specifically” interacting with or binding to means that the ligand more readily interacts with or binds to ASC than to other, non-target proteins. In another embodiment, the ligand does not interact with proteins acting upstream or downstream of a cascade involving ASC. In particular, a “ligand” may not interact with a pH-activated protease, e.g. a pH-activated protease being downstream of the NLRP3 inflammasome. Preferably, a “ligand” is not beta-amyloid. More preferably, the anti-ASC-speck antibody specifically prevents or reduces ASC speck-induced aggregation of Aβ.

A ligand is preferably capable of modulating the biological function or biological activity of its target. The term “biological function” (or “biological activity”) is used herein to refer to the desired or normal effect mediated by said target in a biological (for instance, without limitation, in its natural or native) environment. A ligand “modulates” a biological function of its target if it totally or partially prevents, reduces, inhibits, interferes with, blocks, enhances, activates, stimulates, increases, reinforces or supports said biological function.

A ligand may directly or indirectly interact with its target. Accordingly, the ASC ligand of the present invention may directly or indirectly interact with, preferably bind to, ASC. The ASC ligand of the present invention preferably directly interacts with ASC by (specifically) binding to ASC. However, it is also envisaged that the ASC ligand may indirectly interact with ASC, e.g. by acting upon other cellular or intercellular structures, components or molecules, which affect the biological functions or activities of ASC. The “ligand” may e.g. target ASC by interacting with ASC thereby blocking ASC's interaction with other proteins, e.g. NLRP3.

The term “ASC” refers to the human apoptosis-associated speck-like protein containing a CARD (UniProt Acc. No. Q9ULZ3, entry version #172 of 22 Nov. 2017, sequence version #2) encoded by the PYCARD gene or an allelic variant or ortholog thereof. It may also be referred to as “CARD5” or “TMS1”.

“ASC” preferably comprises or consists of an amino acid sequence corresponding to the amino acid sequence according to SEQ ID NO: 1. This sequence, often referred to as the “canonical” ASC sequence, is depicted below:

```
SEQ ID NO: 1
                10 20 30 40
MGRARDAILD ALENLTAEEL KKFKLKLLSV PLREGYGRIP
                50 60 70 80
RGALLSMDAL DLTDKLVSFY LETYGAELTA NVLRDMGLQE
                90 100 110 120
MAGQLQAATH QGSGAAPAGI QAPPQSAAKP GLHFIDQHRA
              130 140 150 160
ALIARVTNVE WLLDALYGKV LTDEQYQAVR AEPTNPSKMR
              170 180 190
KLFSFTPAWN WTCKDLLLQA LRESQSYLVE DLERS
```

The pyrin domain (PYD) (underlined in the above sequence) is located in the amino acid stretch ranging from amino acids 1-91 (SEQ ID NO: 2). It is considered to mediate homotypic interactions with pyrin domains of proteins such as of NLRP3, PYDC1, PYDC2 and AIM2. The CARD domain (bold in the above sequence) is located in the amino acid stretch ranging from amino acids 107-195 (SEQ ID NO: 3). It is considered to mediate interaction with CASP1 and NLRC4

The term “ASC” preferably also includes homologs, isoforms, variants and fragments of the ASC protein characterized by the amino acid according to SEQ ID NO: 1. These ASC homologs, isoforms, variants and fragments preferably comprise or consist of an amino acid sequence exhibiting a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, as compared to the “canonical” ASC amino acid sequence according to SEQ ID NO: 1.

Such ASC homologs, isoforms, variants and fragments are preferably functional, i.e. retain the biological functions or activities of the ASC protein characterized by the “canonical” ASC amino acid sequence depicted above. Accordingly, such ASC homologs, isoforms, variants and fragments may typically retain at least the minimum parts of the PYD and/or the CARD domain responsible for said biological functions or activities.

ASC is an adaptor protein exhibiting several biological functions or activities. In the context of the present invention, the following functions and activities are of particular interest (typically occurring chronologically from (1)-(5)): (1) capability of being recruited by the NLRP3 inflammasome, typically via pyrin (PYD) domain interactions, (2) helical fibrillar assembly upon NLRP3 recruitment, (3) recruitment of effector caspase-1, typically via CARD interactions, (4) to autoproteolytic activation and subsequent assembly of ASC fibrils into a large paranuclear ASC aggregates (“specks”) and (5) induction of amyloid-R oligomerization and aggregation.

Functional ASC homologs, isoforms, variants and fragments preferably exhibit at least the same biological functions and activities (1)-(5).

The term “amyloid-β” (or “Aβ”, “β-amyloid”, amyloid beta peptide) refers to any one of a group of peptides of 39-43 amino acid residues that are processed from APP. The term “APP” refers to the amyloid-beta A4 protein (Uniprot Ref No. P05067, entry version 8266 pf 22 Nov. 2017) encoded by the APP gene, or a homolog, isoform, variant or fragment thereof. APP is a glycosylated, single-membrane spanning protein expressed in a wide variety of cells in many mammalian tissues. Examples of APP variants which are currently known to exist in humans are the 695 amino acid polypeptide described by Kang et. al. (1987) Nature 325:733-736 (APP695); the 751 amino acid polypeptide described by Ponte et al. (1988) Nature 331:525-527 (1988) and Tanzi et al. (1988) Nature 331:528-530 (SEQ ID NOs:56-57) (APP751); and the 770-amino acid polypeptide described by Kitaguchi et. al. (1988) Nature 331:530-532 (SEQ ID NOs:54-55) (APP770). By convention, the codon numbering of the longest APP protein, APP770, may be used even when referring to codon positions of the shorter APP proteins. APP is processed by secretase cleavage to yield soluble APP or amyloid-R peptides.

The term “amyloid-β” thus refers any peptide resulting from beta secretase cleavage of APP. This includes peptides of 39, 40, 41, 42 and 43 amino acids, extending from the s-secretase cleavage site to 39, 40, 41, 42 and 43 amino acids C-terminal to the R-secretase cleavage site.

ASC ligands according to the present invention are particularly envisaged for treating neurodegenerative diseases in humans. Thus, the ASC ligand is preferably capable of (specifically) interacting with, more preferably binding to, human ASC or its isoforms, variants and fragments. However, it is also envisaged to use the inventive ASC ligand for the treatment of non-human animals. Accordingly, ASC ligands of the present invention may also bind to ASC homologs found in non-human animals.

ASC “homologs” include both “orthologs” and “paralogs”. ASC orthologs include ASC proteins encoded by genes in different species that evolved from a common ancestral gene by speciation (orthologs). Orthologs often retain the same function(s) in the course of evolution. Thus, functions may be lost or gained when comparing a pair of orthologs. ASC paralogs include ASC proteins encoded by genes that were produced via gene duplication within a genome. Paralogs typically evolve new functions or may eventually become pseudogenes.

Exemplary ASC “homologs” include ASC proteins of Gorilla gorilla gorilla (Western lowland gorilla), Nomascus leucogenys (Northern white-cheeked gibbon) (Hylobates leucogenys), Macaca mulatta (Rhesus macaque), Papio anubis (Olive baboon), Cercocebus atys (Sooty mangabey) (Cercocebus torquatus atys), Macaca nemestrina (Pig-tai led macaque), Pan troglodytes (Chimpanzee), Mandrillus leucophaeus (Drill) (Papio leucophaeus), Pongo abelii (Sumatran orangutan) (Pongo pygmaeus abelii) or Colobus angolensis palliates.

ASC ligands according to the present invention may or may not exhibit cross-reactivity to different ASC homologs, i.e. the capability of interacting with or binding to ASC homologs found in two or more different species.

ASC “isoforms” include ASC proteins which differ from the “canonical” ASC protein in terms of their post-translational modifications. Post-translational modifications (PTMs) may result in covalent or non-covalent modifications of a given protein. Common post-translational modifications include glycosylation, phosphorylation, ubiquitinylation, S-nitrosylation, methylation, N-acetylation, lipidation, disulfide bond formation, sulfation, acylation, deamination etc.. Post-translational proteolytic processing may alter the amino acid sequence of a given protein. Different PTMs may result, e.g., in different chemistries, activities, localizations, interactions or conformations, and optionally in different amino acid sequences.

ASC “variants” include ASC protein “sequence variants”, i.e. proteins comprising an amino acid sequence that differs in at least one amino acid residue from a reference (or “parent”) amino acid sequence of a reference (or “parent”) ASC protein. Said reference amino acid sequence may preferably be the canonical amino acid sequence according to SEQ ID NO: 1. ASC variants may thus preferably comprise, in their amino acid sequence, at least one amino acid mutation, substitution, insertion or deletion as compared to the respective reference sequence. Substitutions may be conservative, where wherein amino acids, originating from the same class, are exchanged for one another, or non-conservative. ASC variants include naturally occurring variants, e.g. ASC preproproteins, proproteins, and ASC proteins that have been subjected to post-translational proteolytic processing (this may involve removal of the N-terminal methionine, signal peptide, and/or the conversion of an inactive or non-functional protein to an active or functional one), and naturally occurring mutant ASC proteins. ASC variants further include “transcript variants” (or: “splice variants”). Transcript variants are produced from messenger RNAs that are initially transcribed from the same gene, but are subsequently subjected to alternative (or differential) splicing, where particular exons of a gene may be included within or excluded from the final, processed messenger RNA (mRNA). ASC variants further include engineered ASC variants. It will be noted that ASC “variants” may essentially be defined by an amino acid sequence differing from the amino acid sequence of a reference protein. There may thus be a certain overlap between the terms “variant” and “homolog”, “isoform” (when referring to post-translational modifications altering the amino acid sequence), and “fragment”. A “variant” as defined herein can be derived from, isolated from, related to, based on or homologous to the respective reference protein.

Exemplary ASC“variants” include ASC proteins comprising or consisting of an amino acid sequence corresponding to the amino acid sequence according to SEQ ID NO: 4 or SEQ ID NO: 5.

ASC “fragments” include ASC proteins or (poly-)peptides that consists of a continuous subsequence of the full-length amino acid sequence of a reference (or “parent”) ASC protein. Said reference amino acid sequence may preferably be the canonical amino acid sequence according to SEQ ID NO: 1. A “fragment” is thus, with regard to its amino acid sequence, N-terminally, C-terminally and/or intrasequentially truncated compared to the amino acid sequence of said reference protein. A truncation may occur either on the amino acid level or on the nucleic acid level, respectively. In other words, an ASC protein “fragment” may typically be a shorter portion of a full-length ASC protein amino acid sequence. Accordingly, a fragment, typically, consists of a sequence that is identical to the corresponding stretch within the full-length amino acid sequence. The term includes naturally occurring ASC protein “fragments” (such as fragments resulting from naturally occurring in vivo protease activity) as well as engineered ASC protein fragments.

Preferably, ASC protein “fragments” may consists of a continuous stretch of amino acids corresponding to a continuous stretch of amino acids in the ASC protein amino acid sequence serving as a reference, which represents at least 20%, preferably at least 30%, more preferably at least 40%, more preferably at least 50%, even more preferably at least 60%, even more preferably at least 70%6, and most preferably at least 80% of the reference amino acid sequence. ASC protein “fragments” may comprise or consist of an amino acid sequence of at least 5 contiguous amino acid residues, at least 10 contiguous amino acid residues, at least 15 contiguous amino acid residues, at least 20 contiguous amino acid residues, at least contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of an ASC protein.

ASC homologs, isoforms, variants and fragments according to the invention may preferably exhibit a sequence identity of at least 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99%, preferably of at least 70%, more preferably of at least 80%, even more preferably at least 85%, even more preferably of at least 90% and most preferably of at least 95% or even 97%, with the respective reference amino acid sequence, which is preferably the canonical ASC amino acid sequence according to SEQ ID NO: 1.

The ASC ligand is thus preferably capable of specifically interacting with or binding to an ASC protein as described herein. More preferably, the ASC ligand may be capable of specifically interacting with or binding to an ASC protein characterized by the “canonical” amino acid sequence according to SEQ ID NO: 1, or a homolog, isoform, variant or fragment thereof.

By interacting with or binding to ASC, the ASC ligand according to the present invention is preferably capable of modulating, preferably of totally or partially preventing, reducing, inhibiting, interfering with or blocking the biological functions or activities set out above, i.e. (1) capability of being recruited by the NLRP3 inflammasome, typically via pyrin (PYD) domain interactions, (2) helical fibrillar assembly upon NLRP3 recruitment, (3) recruitment of effector caspase-1, typically via CARD interactions, (4) autoproteolytic activation and subsequent assembly of ASC fibrils into a large paranuclear ASC aggregates (“specks”) and (5) induction of amyloid-β oligomerization and aggregation.

In other words, the ASC ligand according to the present invention preferably acts as an inhibitor of ASC. More preferably, the ASC ligand according to the present invention prevents, reduces, inhibits, interferes with or blocks the capability of ASC to form aggregates (or “specks”) and/or its capability of inducing or promoting amyloid-β aggregation. As used herein, the expression “formation of ASC aggregates” includes the helical fibrillar assembly of ASC (# (2) above) and the assembly of ASC fibrils into a large paranuclear ASC aggregates (# (4) above. The ASC ligand may exert its desired inhibitory action by preventing, reducing, inhibiting, interfering with or blocking any one of the steps of the above-defined functional cascade ultimately inducing the formation of amyloid-β aggregates (#(1)-(5), preferably #(2) and/or #(4) and #(5)). The inhibitory action of an ASC ligand may be assessed by employing the methods described in the appended examples, in particular the Aβ aggregation assay (Example 1).

Accordingly, ASC ligands of the invention may for instance interact with or bind to the PYD or the CARD domain of ASC, in particular an epitope located within the PYD or the CARD domain of ASC. The present inventions report that mutations in the PYD domain, as opposed to mutations in the CARD domain (which are both capable of inhibit ASC helical fibrillar assembly (# (2)) completely prevented the promoting effect of ASC aggregates on amyloid-β aggregation. Without wishing to be bound by specific theory, it is therefore envisaged that ASC ligands according to the invention may (specifically) interact with or bind to the ASC PYD domain, or an epitope located within said domain. Said epitope may include amino acids K21, K22 and/or K26 of the “canonical” ASC amino acid sequence (SEQ ID NO: 1). However, it is likewise conceivable that the ASC ligand interacts with or binds to other parts of the ASC protein, e.g. located in the CARD domain or elsewhere. Such ASC ligands may for instance exert their inhibitory function via sterical interference with PYD interactions, or otherwise.

An “ASC ligand” may be any type of molecule and may preferably be selected from an antibody or a nucleic acid encoding such an antibody, a nucleic acid, a protein, a peptide, an aptamer or a small molecule organic compound. ASC ligands may readily be identified using high-throughput screening or in silico modelling.

Antibody Ligands:

The ASC ligand according to the present invention may an antibody, or a variant, fragment or derivative thereof. The terms “immunoglobulin” (Ig) and “antibody” are used interchangeably herein. The term “antibody” (Ab) as used herein includes monoclonal antibodies, polyclonal antibodies, mono- and multispecific antibodies (e.g., bispecific antibodies), and antibody variants, fragments and derivatives so long as they exhibit the desired biological function, which is typically their binding affinity towards an intended target.

“Binding affinity” or “affinity” is the strength of the binding interaction between a biomolecule (here: ASC) to its ligand/binding partner (here: antibody). Binding affinity is typically measured and reported by the equilibrium dissociation constant (K_{D}). K_{D} is the ratio of k_{off}/kₒₙ, between the antibody and its target. K_{D} and affinity are inversely related. Binding affinity is influenced by non-covalent intermolecular interactions such as hydrogen bonding, electrostatic interactions, hydrophobic and Van der Waals forces between the two molecules. There are many ways to measure binding affinity and dissociation constants, such as ELISA, gel-shift assays, pull-down assays, equilibrium dialysis, analytical ultracentrifugation, SPR, and spectroscopic assays. Isothermal titration calorimetry (ITC). Antibody ASC ligands may exhibit binding affinities in the micromolar (mM), nanomolar (nM), picomolar (pM) or femtomolar fM) range. Antibody ASC ligands may preferably exhibit a high binding affinity towards their intended target. That is, antibody ASC ligands may bind with affinities of at least about 10⁷ M⁻¹, at least about 10⁸ M⁻¹, at least about 10⁹ M⁻¹, at least about 10⁻¹⁰ M⁻¹, at least about 10⁻¹¹ M⁻¹, or at least about 10⁻¹² M⁻¹.

Antibody ASC ligands are preferably capable of specifically interacting with or binding to their intended target. As defined elsewhere herein, the term “specifically binding” means that the antibody binds more readily to its intended target than to a different, non-intended target. An antibody is preferably understood to “specifically bind” or exhibit “binding specificity” or “specific affinity” to its target if it preferentially binds or recognizes the target even in the presence of non-targets as measurable by a quantifiable assay (such as radioactive ligand binding Assays, ELISA, fluorescence based techniques (e.g. Fluorescence Polarization (FP), Fluorescence Resonance Energy Transfer (FRET)), or surface plasmon resonance). An antibody that “specifically binds” to its target may or may not exhibit cross-reactivity to (homologous) targets derived from different species.

The basic, naturally occurring antibody is a heterotetrameric glycoprotein composed of two identical light (L) chains and two identical heavy (H) chains. Some antibodies may contain additional polypeptide chains, such as the J chain in IgM and IgA antibodies. Each L chain is linked to an H chain by one covalent disulfide bond, while the two H chains are linked to each other by one or more disulfide bonds depending on the H chain isotype. Each H and L chain also comprises intrachain disulfide bridges. Each H chain comprises an N-terminal variable domain (V_{H}), followed by three constant domains (C_{H}) for each of the α and γ chains and four C_{H} domains for μ and ε isotypes. Each L chain has at the N-terminus, a variable domain (V) followed by a constant domain at its other end. The V is aligned with the V_{H} and the C_{L} is aligned with the first constant domain of the heavy chain (C_{H}1). Particular amino acid residues are believed to form an interface between the light chain and heavy chain variable domains.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG and IgM, having heavy chains designated α, β, ε, γ and μ, respectively. The γ and μ classes are further divided into subclasses on the basis of relatively minor differences in the CH sequence and function, e.g., humans express the following subclasses: IgG1, IgG2, IgG3, IgG4, IgA1 and IgA2.

The pairing of a V_{H} and V_{L} together forms a single antigen-binding site. The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, the V regions consist of relatively invariant stretches called framework regions (FRs) of about 15-30 amino acid residues separated by shorter regions of extreme variability called “hypervariable regions” also called “complementarity determining regions” (CDRs) that are each approximately 9-12 amino acid residues in length. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen binding site of antibodies. The constant domains are not involved directly in binding an antibody to an antigen, but exhibit various effector functions, such as participation of the antibody dependent cellular cytotoxicity (ADCC). The term “hypervariable region” (also known as “complementarity determining regions” or CDRs) when used herein refers to the amino acid residues of an antibody which are (usually three or four short regions of extreme sequence variability) within the V-region domain of an immunoglobulin which form the antigen-binding site and are the main determinants of antigen binding specificity. CDR residues may be identified based on cross-species sequence variability or crystallographic studies of antigen-antibody complexes.

The term “antibody” as used herein thus preferably refers to immunoglobulin molecules, or variants, fragments or derivatives thereof, which are capable of specifically binding to a target epitope via at least one complementarity determining region. The term includes mono-, and polyclonal antibodies, mono-, bi- and multispecific antibodies, antibodies of any isotype, including IgM, IgD, IgG, IgA and IgE antibodies, and antibodies obtained by any means, including naturally occurring antibodies, antibodies generated by immunization in a host organism, antibodies which were isolated and identified from naturally occurring antibodies or antibodies generated by immunization in a host organism and recombinantly produced by biomolecular methods known in the art, monoclonal and polyclonal antibodies as well as chimeric antibodies, human antibodies, humanized antibodies, intrabodies, i.e. antibodies expressed in cells and optionally localized in specific cell compartments, as well as variants, fragments and derivatives of any of these antibodies.

The term “monoclonal antibody” (mab) as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to “polyclonal” antibody preparations which include different antibodies directed against different epitopes, each monoclonal antibody is directed against a single epitope on the antigen. In addition to their specificity, the monoclonal antibodies are advantageous in that they may be synthesized uncontaminated by other antibodies. The adjective “monoclonal” is not to be construed as requiring production of the antibody by any particular method. For example, the monoclonal antibodies useful in the present invention may be prepared by the hybridoma methodology first described by Kohler et al., Nature 256: 495 (1975), or they may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352: 624-628 (1991) and Marks et al., J. Mol. Biol. 222: 581-597 (1991), for example.

An “antibody variant” or “antibody mutant” refers to an antibody comprising or consisting of an amino acid sequence wherein one or more of the amino acid residues have been modified as compared to a reference or “parent” antibody. Such antibody variants may thus exhibit, in increasing order of preference, at least about 5%, 10%, 20%, 30%, 40%, 50%, 60%, preferably at least about 70%, 80%, 85%, 86%, 87%, 88%, 89%, more preferably at least about 90%, 91%, 92%, 93%, 94%, most preferably at least about 95%, 96%, 97%, 98%, or 99% sequence identity to a reference or “parent” antibody, or to its light or heavy chain. Conceivable amino acid mutations include deletions, insertions or alterations of one or more amino acid residue(s). The mutations may be located in the constant region or in the antigen binding region (e.g., hypervariable or variable region). Conservative amino acid mutations, which change an amino acid to a different amino acid with similar biochemical properties (e.g. charge, hydrophobicity and size), may be preferred.

Antibody variants include “chimeric” and “humanized” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass. “Humanized” antibodies comprising variable domain antigen-binding sequences (partly or fully) derived from a non-human animal, e.g. a mouse or a non-human primate (e.g., Old World Monkey, Ape, etc.), and human constant region sequences, which are preferably capable of effectively mediating Fc effector functions, and/or exhibit reduced immunogenicity when introduced into the human body. “Humanized” antibodies may be prepared by creating a “chimeric” antibody (non-human Fab grafted onto human Fc) as an initial step and selective mutation of the (non-CDR) amino acids in the Fab portion of the molecule. Alternatively, “humanized” antibodies can be obtain directly by grafting appropriate “donor” CDR coding segments derived from a non-human animal onto a human antibody “acceptor” scaffold, and optionally mutating (non-CDR) amino acids for optimized binding.

An “antibody fragment” comprises a portion of an intact antibody (i.e. an antibody comprising an antigen-binding site as well as a C_{L} and at least the heavy chain domains, C_{H}1, C_{H}2 and C_{H}3), preferably the antigen binding and/or the variable region of the intact antibody. Examples of antibody fragments include Fab, Fab′, F(ab′)₂ and Fv fragments.

Papain digestion of antibodies produced two identical antigen-binding fragments, called “Fab” (fragment, antigen-binding) fragments, and a residual “Fc” (fragment, crystallisable) fragment. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen, and a pFc′ fragment. The F(ab′)₂ fragment can be split into two Fab′ fragments. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other antibody fragments and chemical fragments thereof are also known. The Fab/c or Fabc antibody fragment lacks one Fab region. Fd fragments correspond to the heavy chain portion of the Fab and contain a C-terminal constant (C_{H}) and N-terminal variable (V_{H}) domain.

The “Fc” fragment comprises the carboxy-terminal portions of both H chains held together by disulphides. The effector functions of antibodies are determined by sequences in the Fc region, the region which is also recognized by Fc receptors (FcR) found on certain types of cells.

“Fv” is the minimum antibody fragment which contains a complete antigen-binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

An “antibody derivative” is a modified antibody variant that includes a new or additional biological property or functionality. Antibody derivatives may be chemically or biologically modified to introduce desired biological functionalities (e.g. by introducing or removing moieties or domains that confer, enhance, reduce or abolish target binding affinity or specificity or enzymatic activities), manufacturing properties (e.g. by introducing moieties which confer an increased solubility or enhanced excretion, or allow for purification) or pharmacokinetic/pharmacodynamics properties for medical use (e.g. by introducing moieties which confer increased stability, bioavailability, absorption; distribution and/or reduced clearance). For instance, antibody derivatives may be modified to comprise altered glycosylation patterns, or may be conjugated to moieties capable of increasing serum half-life and stability and/or to reduce immunogenicity, such as polyethylene glycol (PEG), dextrans, polysialic acids (PSAs), hyaluronic acid (HA), dextrin, hydroxyethyl-starch (HES), poly(2-ethyl 2-oxazoline) (PEOZ), polypeptides (XTEN technology, PASylation), fatty acids (lipidation) or (additional) antibody Fc parts. Further antibody derivatives include an additional therapeutic moiety, such as a drug, a toxic agent, an enzyme or an adaptor domain. The term “derivative” thus further includes antibody-drug conjugates. Further antibody derivatives include fusion products of antigen-binding antibody regions (CDR and optionally FR regions or antibody V_{L} regions) and other protein domains. An exemplary fusion product is a chimeric antigen receptor (CAR). Further antibody derivatives comprise several antibody fragments typically coupled by a suitable peptide linker. An antibody “derivative” may thus be derived from (and thus optionally include) a naturally occurring (wild-type) antibody, or variants or fragments thereof. Exemplary antibody “derivatives” include diabodies, linear antibodies, single-chain antibodies, and bi- or multispecific antibodies derived from antibody fragments, CARs and antibody-drug conjugates. Combinations of the described modifications are also envisaged herein.

“Single-chain Fv” also abbreviated as “sFv” or “scFv” are antibody derivatives that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding.

The term “diabodies” (also referred to as divalent (or bivalent) single-chain variable fragments, “di-scFvs”, “bi-scFvs”) refers to antibody derivatives prepared by linking two scFv fragments (see preceding paragraph), typically with short linkers (about 5-10) residues) between the V_{H} and V_{L} domains such that inter-chain but not intra-chain pairing of the V domains is achieved. Another possibility is to construct a single peptide chain with two V_{H} and two V_{L} regions (“tandem scFv). The resulting bivalent derivatives have two antigen-binding sites. Likewise, trivalent scFv trimers (also referred to as “triabodies” or “tribodies”) and tetravalent scFv tetramers (“tetrabodies”) can be produced. Di- or multivalent antibody derivatives may be monospecific, i.e. each antigen binding site may be directed against the same target. Such monospecific di- or multivalent antibodies or antibody fragment derivatives preferably exhibit high binding affinities. Alternatively, the antigen binding sites of di- or multivalent antibody derivatives may be directed against different targets, forming bi- or multispecific antibody derivatives.

“Bi- or multispecific” antibody derivatives comprise more than one specific antigen-binding region, each capable of specifically binding to a different target. “Bispecific” derivatives are typically heterodimers of two “crossover” scFv fragments in which the V_{H} and V_{L} domains of two antibodies are present on different polypeptide chains. Bi- or multispecific derivatives may act as adaptor molecules between an effector and a respective target, thereby recruiting effectors (e.g. toxins, drugs, and cytokines or effector cells such as CTL, NK cells, macrophages, and granulocytes) to an antigen of interest, typically expressed by a target cell. Thereby, “bi- or multispecific” derivatives preferably bring the effector molecules or cells and the desired target into close proximity and/or mediate an interaction between effector and target. Bispecific tandem di-scFvs, known as bi-specific T-cell engagers (BiTE antibody constructs) are one example of bivalent and bispecific antibody derivatives.

The structure and properties of antibodies is well-known in the art and described, inter alia, in Janeway's Immunobiology, 9^{th} ed. (rev.), Kenneth Murphy and Casey Weaver (eds), Taylor & Francis Ltd. 2008.

Exemplary ASC ligand antibodies in the context of the present invention may be selected from 653902 clone TMS-1 (BioLegend, San Diego, Calif., U.S.A.); AL177 (AdipoGen, AG-25B-0006-C100, Liestal, Switzerland), LS-C331318-50 (LifeSpan BioSciences); AF3805 (R&D Systems); NBP1-78977 (Novus Biologicals); 600-401-Y67 (Rockland Immunochemicals, Inc.); AF3805-SP (R&D Systems); orb160033 (Biorbyt); orb223237 (Biorbyt); 676502 (BioLegend); 653902 (BioLegend); MBS150936 (MyBioSource.com); MBS420732 (MyBioSource.com); MBS9401386 (MyBioSource.com); MBS9404874 (MyBioSource.com); MBS8504703 (MyBioSource.com); MBS841111 (MyBioSource.com); AB3607 (Merck); 04-147 clone 2EI-7 (Merck); NB300-1056 (Novus Biologicals); NB100-56075 (Novus Biologicals); NBP1-78978 (Novus Biologicals); NBP1-78977SS (Novus Biologicals); NBP1-78978SS (Novus Biologicals); NBP1-77297 (Novus Biologicals); AP07343PU-N (OriGene Technologies); AP06792PU-N (OriGene Technologies); AM26452AF-N (OriGene Technologies); AP32825PU-N (OriGene Technologies); AP23602PU-N (OriGene Technologies); TA306044 (OriGene Technologies); 3291-100 (BioVision); 3291-30T (BioVision); STJ25245 (St John's Laboratory); STJ91730 (St John's Laboratory); LS-C180180-100 (LifeSpan BioSciences); LS-C48292-100 (LifeSpan BioSciences); STJ70108 (St John's Laboratory); STJ113135 (St John's Laboratory); LS-C155196-100 (LifeSpan BioSciences); GTX22236 (GeneTex); GTX102474 (GeneTex); GTX28394 (GeneTex); D086-3 (MBL International); 13833S (Cell Signaling Technology); CAE04552 (Biomatik); ADI-905-173-100 (Enzo Life Sciences, Inc.); 40618 (Signalway Antibody LLC); E-AB-30582 (Elabscience Biotechnology Inc.); ab180799 (Abcam); 168-10230 (Raybiotech, Inc.); ER-03-0001 (Raybiotech, Inc.); A3598-05B-100ug (United States Biological); A3598-05N-50ug (United States Biological); AP5631 (ECM Biosciences); ABIN1001824 (antibodies-online); 2287 (ProSci, Inc); 70R-11744 (Fitzgerald Industries International); AHP1606 (Bio-Rad); PA1-41405 (Invitrogen Antibodies); PA5-19957 (Invitrogen Antibodies); PA5-27715 (Invitrogen Antibodies); PA1-9010 (Invitrogen Antibodies); 10500-1-AP (Proteintech Group Inc); sc-514414 (Santa Cruz Biotechnology, Inc.); and sc-514559 (Santa Cruz Biotechnology, Inc.).

The ASC ligand of the present invention may be chosen from any one of the above-mentioned antibodies, or a variant (such as a humanized or otherwise engineered variant), fragment (such as a Fab or Fv fragment) or derivative (such as scFvs or diabodies) thereof. Means and methods for providing such variants, fragments or derivatives are known in the art and are interalia described in Kontermann, Roland; Dbel, Stefan (Eds.) Antibody Engineering Series: Springer Lab Manuals 2001, ISBN: 978-3-540-41354-7. Fragments or derivatives prepared from humanized antibody variants are particularly envisaged herein.

Protein or Peptide ASC Ligands:

The ASC ligand according to the present invention may be selected from a protein or peptide. Protein or peptide ASC ligands are preferably binding proteins or peptides other than antibodies or their variants, fragments or derivatives, which exhibit a specific affinity towards ASC.

Protein or peptide ligands typically comprise a binding domain mediating the (specific) interaction with or binding to ASC. Such binding domains may comprise or be derived from FN3 (fibronectin type III domain), β-sheet frameworks, Kunitz domains, PDZ domain (PSD-95/Discs-large/ZO-1-domains), human A-domains, repeat domains (such as ankyrin repeat domains) or staphylococcal protein A (SPA), as reviewed in Grönwall S and Stahl. Journal of Biotechnology. 140 (2009): 254-269.

Protein or peptide ASC ligands include naturally occurring proteins and peptides as well as engineered variants and derivatives thereof.

The terms “specific affinity”, “variant” and “derivative” are explained in the context of antibody ASC ligands and are, mutatis mutandis, equally applicable to protein or peptide ASC ligands. As such, derivatives include for instance chimeric fusions including a first amino acid sequence (protein) fused to (optionally via a suitable peptide linker) a second amino acid sequence defining a domain foreign to and not substantially homologous with any domain of the first protein. The domains may or may not be derived from different species.

Exemplary protein or peptide ASC ligands include soluble receptors, adnectins, anticalins, DARPins (designed ankyrin repeat proteins), avimers, affibodies, peptide aptamers or variants, fragments or derivatives thereof.

Nucleic Acid ASC Ligands:

The ASC ligand according to the present invention may be selected from a nucleic acid.

Nucleic acid ASC ligands may be single-stranded or double-stranded or mixtures thereof, and include DNA and RNA molecules. Nucleic acid ASC ligands may be of any length. They may or may not include modified nucleosides, nucleotides or phosphodiester linkages. Nucleic acid ASC ligands may be coding or non-coding.

Nucleic acid ASC ligands may be binding nucleic acids, which exhibit a specific affinity towards ASC. The term “specific affinity” is explained in the context of antibody ASC ligands and is, mutatis mutandis, equally applicable to nucleic acid ASC ligands. Such nucleic acid ASC ligands may be selected from aptamers.

“Aptamers” or “oligonucleotide aptamers” are small nucleic acid ligands composed of RNA or single-stranded DNA oligonucleotides which fold into three-dimensional (3D) structures. Aptamers interact with and bind to their targets through structural recognition, a process similar to that of an antigen-antibody reaction. The term “aptamer” as used herein includes mono-, bi- and polyvalent aptamers, mono-, bi- and multispecifc aptamers, aptamer-drug conjugates (ApDC) comprising aptamers covalently coupled to a drug, optionally via a suitable linker, aptamers coupled to high molecular weight polymers (e.g. PEG), aptamer-tethered DNA nanotrains (aptNTrs), aptamers associated with carriers (e.g. copolymers, liposomes metal nanoparticles or virus-like particules), aptamer-Fc conjugates and aptamer-siRNA or aptamer-miRNA chimeras. (cf. Sun et al. Molecular Therapy Nucleic Acids (2014) 3, e182 for review).

Alternatively, nucleic acid ASC ligands may indirectly interact with ASC function and activities by e.g. modulating ASC expression. Such nucleic acid ASC ligands may be selected from microRNAs, siRNAs, shRNAs or antisense RNAs.

“MicroRNAs” or “miRNAs” are small (˜20-24 nucleotide) non-coding double-stranded RNAs (dsRNAs) capable of recruiting the AGO-2 RISC complex to a complementary target transcript, thereby preferably inducing the miRNA-mediated RNAi pathway. MicroRNAs are typically processed from pri-microRNA to short stem-loop structures called pre-microRNA and finally to mature miRNA. Both strands of the stem of the pre-microRNA may be processed to a mature microRNA. After processing, the mature single-stranded microRNAs, associated with Argonaute 2 (AGO2) in the RNA-induced silencing complex (RISC), typically bind to the 3′ UTRs of their cytosolic mRNA targets, resulting in either reduced translation or deadenylation and degradation of the mRNA transcript. The predominant function of microRNAs is thus to (negatively) regulate protein translation by binding to complementary sequences of target mRNAs. The term “microRNA” includes miRNAs, mature single stranded miRNAs, precursor miRNAs (pre-miRNA), primary miRNA transcripts (pri-miRNA), duplex miRNAs and variants thereof. MicroRNAs are particularly envisaged to be capable of binding to a target site within a 3′, untranslated region of a target nucleic acid.

“Small interfering RNAs” or “siRNAs” are small (˜12-35 nucleotide) non-coding RNA molecules capable of inducing RNAi. siRNAs comprise an RNA duplex (double-stranded region) formed by complement base pairing with phosphorylated 5′-ends and hydroxylated 3′-ends, optionally with one or two single-stranded overhanging nucleotides. The duplex portion typically comprises between 17 and 29 nucleotides. siRNA may be generated from two RNA molecules that hybridize together or may alternatively be generated from a single RNA molecule that includes a self-hybridizing portion (shRNA). The duplex portion of an siRNA may, but typically does not, include one or more bulges containing one or more unpaired and/or mismatched nucleotides in one or both strands of the duplex or may contain one or more non-complementary nucleotide pairs. One strand of a siRNA (referred to as the antisense strand) includes a portion that hybridizes with a target transcript (e.g. a target mRNA). The antisense strand may be precisely complementary with a complementary region of the target transcript (i.e. the siRNA antisense strand may hybridize to the target sequence without a single mismatch, wobble base pairing or nucleotide bulge) or one or more mismatches, wobble (G:U) base pairings and/or nucleotide bulges between the siRNA antisense strand and the complementary region of the target transcript may exist.

“Short hairpin RNAs” or “shRNAs” are single-strand RNA molecules comprising at least two complementary portions hybridized or capable of hybridizing to form a double-stranded (duplex) structure sufficiently long to mediate RNAi. These complementary portions are generally between 17-29 nucleotides in length, typically at least 19 base pairs in length. shRNAs further comprise at least one single-stranded portion, typically between 1-10 nucleotides in length that forms a loop connecting the complementary strands forming the duplex portion. The duplex portion may, but typically does not, contain one or more bulges consisting of one or more unpaired nucleotides. As described above, shRNAs are thought to be processed into siRNAs (see above) by the RNAi machinery. shRNAs are therefore siRNA precursors and are thought to induce gene silencing via the siRNA-mediated RNAi pathway.

“Antisense RNAs” or “asRNAs” are single or double-stranded RNA molecules exhibiting preferably at least 90%, more preferably 95% and especially 100% (of the nucleotides of a dsRNA) sequence identity to a section of a naturally occurring mRNA sequence. In the context of the present invention, such naturally occurring mRNA sequence may be coding for ASC. Antisense RNAs typically exhibit complementarity either to a coding or a non-coding section, however, in some cases wobble base (G:U) pairing, nucleotide bulges and/or mismatches may occur as long as they do not abolish the capability of the antisense RNA to bind to its target.

Small Molecule ASC Ligands:

The ASC ligand according to the present invention may be selected from a small organic molecule. Said small organic molecule may preferably exhibit a specific affinity towards ASC. The term “specific affinity” is explained in the context of antibody ASC ligands and is, mutatis mutandis, equally applicable to small organic molecule ASC ligands.

The term “small organic molecule ASC ligand” includes any small organic molecule compound capable of directly or indirectly interacting with ASC, and pharmaceutically acceptable salts, esters, derivatives, analogues and mimetic compounds thereof.

Preferably, the ASC interacting compound is not an ester compound, more preferably not caffeic acid phenylester (CAPE), CAPEN, DHC or DMC, in particular not CAPE.

### Nucleic Acid Molecules Encoding ASC Ligands

In a further aspect, the present invention provides nucleic acid molecules encoding ASC ligands—such as antibody, protein, peptide or nucleic acid ligands—described herein. A nucleic acid molecule “encoding” an ASC ligand is capable of being expressed to provide said ligand under appropriate conditions. Nucleic acid molecules may be single-stranded or double-stranded or mixtures thereof, and include DNA and RNA molecules. Exemplary nucleic acid molecules may be selected from constructs, genomic DNA including sense and antisense DNA, complementary DNA (cDNA), heterogeneous nuclear RNA (hnRNA), precursor mRNA (pre-mRNA), (mature) messenger RNA (mRNA), DNA:RNA hybrid molecules, mini-genes, and gene fragments.

The nucleic acid molecule of the invention may be of any length. The nucleic acid molecule of the invention may comprise natural nucleosides (e.g., adenosine, thymidine, guanosine, cytidine, uridine, deoxyadenosine, deoxythymidine, deoxyguanosine, and deoxycytidine), nucleoside analogues (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), and/or nucleosides comprising chemically or biologically modified bases, (e.g., methylated bases), intercalated bases, and/or modified sugars (e.g., 2′-fluororibose, ribose, 2′-deoxyribose, arabinose, and hexose). The nucleic acid molecule of the invention may comprise phosphodiester linkages, or any other type of linkage such as phosphorothioate and 5′-N-phosphoramidite linkages. Nucleic acid molecules comprising non-naturally occurring nucleosides or nucleotides, sequences, backbones or internucleotide linkages are also referred to as “modified” nucleic acid molecules herein.

Nucleic acid molecules of the invention may be obtained by using biological means (e.g., enzymatically) in vivo or in vitro, or may be chemically synthesized.

The nucleic acid molecule according to the invention is characterized by its polynucleotide sequence. Said sequence preferably comprises a “coding region” or “coding sequence” (cds) encoding the ASC ligand of interest. As used herein, the term “encoding” means being capable of being expressed to provide a desired expression product (such as a protein, peptide or nucleic acid) in an appropriate environment, such as a suitable host cell or under suitable conditions in vitro. The polynucleotide sequence of the open reading frame encoding the ASC may be readily isolated from a genomic DNA source, a cDNA source, or may be synthesized (e.g., via PCR).

The nucleic acid molecule of the invention may thus comprise or consist of a cds encoding a (proteinaceous) ASC ligand described herein, and optionally regulatory elements operably linked thereto.

The term “operably linked” refers to the linkage of a polynucleotide sequence to another polynucleotide sequence in such a way as to allow the sequences to function in their intended manner. A protein-encoding polynucleotide sequence is for example “operably linked” to a regulatory element when it is connected to said element in a functional manner which allows the expression of said polynucleotide sequence to yield the encoded protein.

The terms “regulatory element” and “regulatory sequence” are used interchangeably and refer to polynucleotide sequences capable of modulating the biological function or activity of an operably linked polynucleotide sequence in a host cell. Regulatory elements for instance include sequences capable of directing or modulating (e.g. increasing) the expression of a protein from a protein-encoding polynucleotide sequences. The term thus covers elements that promote or regulate transcription, including promoters, core elements required for basic interaction of RNA polymerase and transcription factors, splicing signals, polyadenylation signals, upstream elements, enhancers, and response elements. Regulatory elements that are capable of directing expression in prokaryotes include promoters, operator sequences and ribosome binding sites. Regulatory elements may be of genomic (e.g. viral or eukaryotic) origin or may be synthetically generated. Regulatory elements may be derived from libraries or databases and chemically synthesized. Regulatory elements may be introduced into the nucleic acid molecules of the invention to optimize transcription, mRNA processing and stabilization and translation into the encoded amino acid sequence.

Regulatory elements may be linked to polynucleotide sequences of interest by ligation at suitable restriction sites or via adapters or linkers inserted into the sequence using restriction endonucleases known to one of skill in the art.

“Promoters” or “promoter sequences” are nucleotide sequences located at the transcription initiation site (typically upstream or 5′ of the site of transcription initiation) and initiate transcription of a particular polynucleotide sequence of interest. Promoters may either be constitutive or inducible. Inducible promoters initiate the transcription of operably linked cds only under certain physiological conditions and may be controlled depending upon the host cell, the desired level of expression, the nature of the host cell, and the like.

Promoters include eukaryotic promoters, viral promoters and synthetic promoters, e.g. the β-actin promoter, SV40 early and late promoters, immunoglobulin promoter, human cytomegalovirus (CMV) promoter, retrovirus promoters, and others. The promoter may or may not be associated with enhancers, wherein the enhancers may be naturally associated with the particular promoter or associated with a different promoter.

The term “enhancer” refers to a cis-acting nucleotide sequence, which enhances the transcription of an operably linked polynucleotide sequence and functions in an orientation- and position-independent manner. The enhancer may function in any location, either upstream or downstream relative to the transcription initiation site. The enhancer may comprise or consist of any nucleotide sequence capable of increasing the level of transcription from the promoter when the enhancer is operably linked to the promoter. Exemplary enhancers include the RSV LTR enhancer, baculovirus HR1, HR2 or HR3 enhancers or the CMV immediate early gene product enhancer.

A “marker” may be introduced into the nucleic acid molecule of the invention in order to enable the detection or selection of host cells that have been successfully transformed with (i.e. comprise) the nucleic acid molecule and/or vector of the invention. A marker is typically a gene, which, upon being introduced into the host cell, expresses a dominant phenotype permitting positive selection or detection of cells carrying the gene. Genes of this type are known in the art, and include, inter a/ia, green fluorescent protein (GFP), yellow fluorescent protein (YFP), red fluorescent protein (RFP), luciferase, beta-galactosidase (beta-Gal), beta-glucuronidase, hygromycin-B phosphotransferase gene (hph), the aminoglycoside phosphotransferase gene (neo or aph), the dihydrofolate reductase (DHFR) gene, the adenosine daminase gene (ADA), and the multi-drug resistance (MDR) gene.

Further regulatory elements of interest include an “origin of replication” (“ori”), which confers the ability to replicate in a desired host cell. Optionally, the nucleic acid molecule may comprise regulatory elements, which effect ligation or insertion into a desired host cell.

### Vector

In a further aspect, the present invention provides a vector comprising the nucleic acid molecule according to the invention. In other words, the present invention provides a vector comprising a polynucleotide sequence encoding an ASC ligand—such as an antibody—as described herein.

A “vector” (also referred to herein as a “vehicle,” or “construct”) is a nucleic acid molecule serving as a vehicle for the transfer, expression, replication, multiplication, integration and/or storage of a polynucleotide sequence of interest.

Vectors according to the present invention may be selected from viral or non-viral vector.

Non-viral vectors include plasmids (integrating or non-integrating), plasmid mini-circles, transposons, cosmids and artificial chromosomes, such as bacterial artificial chromosomes (BACs) and yeast artificial chromosomes (YACs). Such non-viral vectors may be complexed with polymers or lipids or can be provided in the form of “naked” RNA or DNA.

Viral vectors include retroviruses, herpes viruses, lentiviruses, adenoviruses and adeno-associated viruses. Retroviruses, lentiviruses and adeno-associated viruses integrate into host cell DNA and therefore have potential for long term expression in the host. Retroviruses may be selected from murine leukaemia virus (MLV), mouse mammary tumour virus (MMTV), Rouse sarcoma virus (RSV), Moloney murine leukaemia virus (Mo MLV), Fujinami sarcoma virus (FuSV), Moloney murine sarcoma virus (Mo-MSV), Abelson murine leukaemia virus (A-MLV) and Avian erythroblastoma virus (AEV). Lentiviruses may be selected from human immunodeficiency virus (HIV), simian immunodeficiency virus (SIV), feline immunodeficiency virus (FIV), equine infectious anaemia virus (EIAV), caprine arthritis encephalitis virus (CAEV), bovine immunodeficiency virus (BIV) and Jembrana disease virus (JDV) based vectors. Adenoviruses may be selected from adenovirus type 5 first and second generation and gutless vectors. Adeno-associated viruses may be selected from all adeno-associated serotypes.

The vector according to the present invention may be integrated into the host cell's genome or exist as an independent genetic element (e.g., episome, plasmid). The vector may exist as a single nucleic acid molecule or as two or more separate nucleic acid molecules. The vector may be a single copy vector or a multicopy vector (indicating the number of copies of the vector typically maintained in the host cell). Vectors are typically recombinant, i.e. artificial molecules which do not occur in nature. The vector may be present in linear and/or in circular form. Some circular nucleic acid vectors may intentionally be linearized prior to delivery into a cell.

The polynucleotide sequence encoding the inventive ASC ligand may be inserted into the vector “backbone” using known methods in the art (cf. Sambrook j et al., 2012 (4th ed.), Molecular cloning: a laboratory manual. Cold Spring Harbor Laboratory, Cold Spring Harbor, N.Y.). These methods may include in vitro recombinant DNA and synthetic techniques and genetic recombination. The resulting vector is referred to as a “recombinant” vector because it comprises novel combinations of nucleic acid sequences from the donor genome with the vector nucleic acid sequence. Recombinant vectors comprising the desired polynucleotide sequence may be identified by known techniques including (a) sequencing (b) nucleic acid hybridization; (c) presence or absence of “marker” gene functions; and (d) expression of the inserted polynuleoctide sequences.

The vector may comprise additional regulatory elements in its “backbone”, e.g. an origin of replication, enhancers, restriction sites, or regulatory elements as described elsewhere herein. The vector may comprise regulatory sequences directing its ligation and integration into the host cell genome etc.

Vectors according to the present invention may be selected from storage vectors, cloning vectors, transfer or shuttle vectors, expression vectors, gene therapy vectors and other vectors. As will be readily understood, the above definitions may overlap to a certain degree, e.g. some transfer vectors can also function as expression vectors.

Preferably, the vector according to the invention may be a gene therapy vector or an expression vector.

An “expression vector” is a vector that is capable of effecting the expression of an encoded expression product. “Expression vectors” are typically recombinant nucleic acid molecules comprising one or more polynucleotide sequences encoding an expression product of interest in the form of an “expression cassette”. An “expression cassette” comprises said polynucleotide sequence(s) and appropriate regulatory elements promoting the efficient transcription of said polynucleotide sequence(s). It is typically inserted into a multiple cloning site in the vector backbone Suitable regulatory elements may include transcriptional promoters and optionally enhancers, translational signals, and transcriptional and translational termination signals. The choice of expression vector will be influenced by the choice of the host expression system. Expression vectors that are used for stable transformation typically have a selectable marker which allows selection and maintenance of the transformed cells. In some cases, an origin of replication can be used to amplify the copy number of the vector.

A “gene therapy vector” is a vector that can be transferred to a subject to be treated where it effects the expression of polynucleotide sequence.

### Host Cell

In a further aspect, the present invention provides a host cell comprising the ASC ligand, the nucleic acid molecule and/or the vector according to the invention.

The choice of suitable host cells depends on their desired use and function.

The present invention inter alia envisages host cells for expressing the polynucleotide sequences of the nucleic acid molecules and/or vectors encoding ASC ligands according to the present invention. A variety of host-vector systems can be used to express the polynucleotide sequence encoding the ASC ligand. These include mammalian cell systems infected with virus (e.g. vaccinia virus, adenovirus and other viruses); insect cell systems infected with virus (e.g. baculovirus); microorganisms such as yeast containing yeast vectors; or bacteria transformed with bacteriophage, DNA, plasmid DNA, or cosmid DNA.

More specifically, host cells may be selected from prokaryotic cells, yeast cells, insect cells, plant cells or mammalian cells.

Prokaryotic cells, such as E. coli, may be used for producing large amounts of (proteinaceous) ASC ligands. Transformation of E. coli is simple and rapid technique well known to those of skill in the art. Expression vectors for E. coli may contain inducible promoters, e.g. for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated APL promoter.

(Proteinaceous) ASC ligands may be expressed in the cytoplasmic environment of E. coli. Alternatively, a leader sequence may be fused to the desired protein product in order to direct the protein into the oxidizing periplasmatic space. The leader is typically removed by signal peptidases inside the periplasm. Examples of periplasmic-targeting leader sequences include the pelB leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Reducing agents such as dithiothreitol and β-mercaptoethanol and denaturants, such as guanidine-HCl and urea may be used to increase solubility of the expressed protein products. Temperature of induction and growth also can influence expression levels and solubility, typically temperatures between 25° C. and 37° C. are used. Typically, bacteria produce aglycosylated proteins. Thus, if proteins require glycosylation for function, glycosylation can be added in vitro after purification from host cells.

Yeast cells such as Saccharomyces cerevisae, Schizosaccharomyces pombe, Yarrowia lipolytica, Kluyveromyces lactis and Pichia pastoris are well known yeast expression hosts that can be used for production of proteins, such the (proteinaceous) ASC ligands described herein. Yeast cells may be transformed with episomal replicating vectors or by stable chromosomal integration by homologous recombination. Typically, inducible promoters are used to regulate gene expression. Examples of such promoters include GAL1, GAL7 and GAL5 and metallothionein promoters, such as CUP1, AOX1 or other Pichia or other yeast promoters. Expression vectors may include a selectable marker such as LEU2, TRP1, HIS3 and URA3 for selection and maintenance of the transformed DNA. Proteins expressed in yeast are often soluble. Co-expression with chaperonins such as Bip and protein disulfide isomerase may improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site such as for the Kex-2 protease, may be engineered to remove the fused sequences from the expressed polypeptides as they exit the secretion pathway. Yeast also is capable of glycosylation at Asn-X-Ser/Thr motifs.

Insect cell expression systems express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter of baculovirus. Commonly used baculovirus systems include the baculoviruses such as Autographa californica nuclear polyhedrosis virus (AcNPV), and the Bombyx mori nuclear polyhedrosis virus (BmNPV) and an insect cell line such as Sf9 derived from Spodoptera frugiperda, Pseudaletia unipuncta (A7S) and Danaus plexippus (DpN1). For high-level expression, the nucleotide sequence of the molecule to be expressed may be fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schneider 2 (S2) and Kc cells (Drosophila melanogaster) and C7 cells (Aedes albopictus) may be used for expression. The Drosophila metallothionein promoter can be used to induce high levels of expression in the presence of heavy metal induction with cadmium or copper. Expression vectors are typically maintained by the use of selectable markers such as neomycin and hygromycin.

Expression vectors may be transferred to mammalian cells by viral infection such as adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. IRES elements also can be added to permit bicistronic expression with another gene, such as a selectable marker. Such vectors often include transcriptional promoter-enhancers for high-level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase 1, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase (DHFR) and thymidine kinase. For example, expression can be performed in the presence of methotrexate to select for only those cells expressing the DHFR gene. Fusion with cell surface signaling molecules such as TCR-ζ and FcεRI-γ can direct expression of the proteins in an active state on the cell surface.

Many cell lines are available for mammalian expression including mouse, rat human, monkey, chicken and hamster cells. Exemplary cell lines include but are not limited to CHO, Balb/3T3, HeLa, MT2, mouse NSO (nonsecreting) and other myeloma cell lines, hybridoma and heterohybridoma cell liries, lymphocytes, fibroblasts, Sp2/0, COS, NIH3T3, HEK293, 293S, 2B8, and HKB cells. Cell lines also are available adapted to serum-free media which facilitates purification of secreted proteins from the cell culture media. Examples include CHO-S cells (Invitrogen, Carlsbad, Calif., cat #11619-012) and the serum free EBNA-1 cell line (Pham et al., (2003) Biotechnol. Bioeng. 84:332-342). Cell lines also are available that are adapted to grow in special mediums optimized for maximal expression. For example, DG44 CHO cells are adapted to grow in suspension culture in a chemically defined, animal product-free medium.

Transgenic plant cells and plants can be used to express proteins such as any described herein. Expression vectors are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline synthetase promoter, the ribose bisphosphate carboxylase promoter and the ubiquitin and UBQ3 promoters. Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce hyaluronidase polypeptides. Because plants have different glycosylation patterns than mammalian cells, this can influence the choice of protein produced in these hosts.

The (proteinaceous) ASC ligand may be purified from host cells using any suitable technique known in the art. Secreted proteins are typically purified from the culture media after removing the cells. Intracellularly expressed proteins are typically purified from cellular extracts after host cell lysis. Purification techniques may involve SDS-PAGE, size fraction and size exclusion chromatography, ammonium sulfate precipitation and ionic exchange chromatography, such as anion exchange. Affinity purification techniques may also be utilized to purify antibodies or other proteins or peptides. Said antibodies or proteins or peptides may also be engineered to add an affinity tag such as a myc epitope, GST fusion or Hiss and enable affinity purification with myc antibody, glutathione resin and Ni-resin, respectively. Purity may be assessed by any method known in the art including gel electrophoresis and staining and spectrophotometric techniques.

### (Pharmaceutical) Composition

In a further aspect, the present invention provides a composition comprising at least one of the ASC ligands, nucleic acids, vectors or host cells described herein, or a combination thereof, and optionally at least one pharmaceutically acceptable excipient. The composition may preferably be a pharmaceutical composition. Pharmaceutical compositions are typically prepared in view of approvals for a regulatory agency or other agency prepared in accordance with generally recognized pharmacopeia for use in animals and in humans.

Excipients may be added for the purpose of production enhancement, patient acceptability, improving stability, controlling release etc. Typically excipients are the major components of a pharmaceutical composition, with the active agent only present in relatively small amounts. Sometimes, excipients are also referred to as “inactive” or “inert” components. However, some excipients may also have an impact on the pharmacokinetics or pharmacodynamics, and in particular on absorption, distribution, metabolism and elimination (ADME) processes of the co-administered active agent.

Excipients are typically classified based on their role in the pharmaceutical formulation and on their interactions influencing drug delivery, based on their chemical and physico-chemical properties. Main classes of excipients include antioxidants, coating materials, emulgents, taste- and smell-improvers, ointment bases, conserving agents, consistency-improvers, distintegrating materials, diluents, fillers, bulking material, carriers, binders, lubricants, glidants, solvents and co-solvents, buffering agents, wetting agents, anti-foaming agents, thickening agents and humectants. Some excipients may serve multiple purposes; for example, methylcellulose is a coating material, is applied in the preparation of suspensions, to increase viscosity, as a disintegrating agent or binder in tablets.

The term “pharmaceutically acceptable” refers to a compound that is compatible with the one or more active agent(s) and does not interfere with and/or substantially reduces its/their pharmaceutical effect. Pharmaceutically acceptable excipients preferably have sufficiently high purity and sufficiently low toxicity to make them suitable for co-administration with the active agent(s) to a subject.

The choice of suitable pharmaceutically acceptable excipients is typically determined by the chosen route of administration and formulation of the pharmaceutical composition.

Pharmaceutical compositions according to the invention may be administered via subcutaneous, intravenous, intramuscular, intraarterial, intradermal, intraperitoneal, intravascular (i.v.), intranasal, transdermal, intralesional, intratumoral, intracranial, intrapulmonal, intracardial, sublingual, rectal, buccal or vaginal administration routes. Formulations suited for such routes are known to one of skill in the art. Administration may be local or systemic. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant. Systemic administration can be achieved by oral administration or by injection, which may be needle-free injection (jet injection) and/or needle injection.

Pharmaceutical compositions may be formulated as tablets, capsules, pills, powders, granules, suppositories, sterile parenteral solutions or suspensions, oral solutions or suspensions, oil water emulsions and sustained release formulations. The formulation should suit the mode of administration. Pharmaceutical compositions according to the invention may also be provided as lyophilized powders, which can be reconstituted for administration as solutions, emulsions and other mixtures. They may also be reconstituted and formulated as solids or gels.

Pharmaceutical compositions for topical administration may be formulated as creams, gels, ointments, emulsions, solutions, elixirs, lotions, suspensions, tinctures, pastes, foams, aerosols, irrigations, sprays, suppositories, bandages, dermal patches, aerosols or any other formulations suitable for topical administration. Pharmaceutical compositions for rectal administration may be formulated as rectal suppositories, capsules and tablets. Pharmaceutical compositions for oral administration may be formulated as tablets or capsules. Pharmaceutical compositions may also be administered by controlled release formulations and/or delivery devices. (Pharmaceutical) compositions according to the invention may be formulated in liquid, solid or semisolid form.

Pharmaceutical compositions according to the invention may be provided in unit dosage forms or multiple dosage forms. Each unit dose typically contains an effective amount of the active agent(s), together with the required pharmaceutically acceptable excipient. Examples of unit dose forms include ampoules and syringes and individually packaged tablets or capsules. Unit dose parenteral formulations can be packaged in, for example, an ampoule, a cartridge, a vial or a syringe with a needle. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, a multiple dose form is a multiple of unit doses that are not segregated in packaging.

It may be preferred to administer the inventive ASC ligands parenterally.

Parenteral administration may be accomplished by injection or infusion, e.g. subcutaneous, intramuscular, intravenous or intradermal injection or infusion. Alternatively, parenteral administration can be achieved by inhalation. Pharmaceutical compositions for parenteral administration may be prepared as liquid solutions or suspensions, emulsions, or in solid forms capable of being reconstituted in a suitable liquid medium prior to administration. Pharmaceutical compositions for parenteral administration are typically stored in vials, IV bags, ampoules, cartridges, or prefilled syringes.

Pharmaceutical compositions for parenteral administration include preferably sterile solutions, suspensions or emulsions ready for administration, or concentrated forms thereof which have to be diluted in a suitable solvent prior to use. Solutions, suspensions and emulsions may be either aqueous or nonaqueous.

Examples of aqueous vehicles include Sodium Chloride Injection, Ringers Injection, Isotonic Dextrose Injection, Sterile Water Injection, Dextrose and Lactated Ringers Injection. Nonaqueous vehicles include fixed oils of vegetable origin, almond oil, oily esters, cottonseed oil, corn oil, sesame oil and peanut oil. Liquid pharmaceutical compositions may further comprise buffering agents, wetting agents, emulsifying agents, stabilizers, solubility enhancers, antimicrobial agents, isotonic agents, antioxidants, local anesthetics, suspending and dispersing agents, emulsifying agents, sequestering or chelating agents. Antimicrobial agents in bacteriostatic or fungistatic concentrations include phenols or cresols, mercurials, benzyl alcohol, chlorobutanol, methyl and propyl p-hydroxybenzoic acid esters, thimerosal, benzalkonium chloride, sorbic acid and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN 80). A sequestering or chelating agent of metal ions include EDTA or cyclodextrins. Thickening and solubilizing agents include glucose, polyethylene glycol, and polypropylene glycol. Suspending agents include sorbitol syrup, cellulose derivatives or hydrogenated edible fats. Emulsifying agents include lecithin or acacia. Further excipients of interest include polyethylene glycol and propylene glycol for water miscible vehicles; sodium hydroxide, hydrochloric acid, citric acid or lactic acid for pH adjustment.

Liquid pharmaceutical compositions for intravenous administration are preferably sterile and isotonic. Suitable excipients include preferably sterile and isotonic aqueous vehicles such as physiological saline or phosphate buffered saline (PBS) as carriers and thickening or solubilizing agents such as glucose, polyethylene glycol, and polypropylene glycol.

Pharmaceutical compositions may comprise delivery systems such as liposomes, lipid nanoparticles, lipoplexes, microparticles or microcapsules.

Pharmaceutical compositions may further comprise additional active agents useful for treating or preventing the neurodegenerative diseases defined herein. Such additional active agents may be selected from nootropic agents, neuroprotectants, antiparkinsonian drugs, amyloid protein deposition inhibitors, beta amyloid synthesis inhibitors, antidepressants, anxiolytic drugs, antipsychotic drugs and anti-multiple sclerosis drugs, or combinations thereof.

### Kit

In a further aspect, the present invention relates to a kit or kit-of-parts comprising the ASC ligand, nucleic acid, vector, host cell, pharmaceutical composition according to the invention, or any combination thereof. Optionally, the kit may additionally comprise pharmaceutically acceptable excipients or further active agents as described in the section “(Pharmaceutical) composition”.

The ASC ligand, nucleic acid, vector, host cell or pharmaceutical composition may be provided in any suitable form, e.g. in liquid or lyophilized form.

The kit or kit-of-parts may be a kit of two or more parts and typically comprises its components in suitable containers. For example, each container may be in the form of vials, bottles, squeeze bottles, jars, sealed sleeves, envelopes or pouches, tubes or blister packages or any other suitable form provided the container is configured so as to prevent premature mixing of components. Each of the different components may be provided separately, or some of the different components may be provided together (i.e. in the same container).

A container may also be a compartment or a chamber within a vial, a tube, a jar, or an envelope, or a sleeve, or a blister package or a bottle, provided that the contents of one compartment are not able to associate physically with the contents of another compartment prior to their deliberate mixing by a pharmacist or physician.

The kit or kit-of-parts may furthermore contain technical instructions with information on the use, administration and dosage of any of its components.

### Medical Use and Treatment

In a further aspect, the present invention relates to a method of treating a neurodegenerative disease comprising administering an effective amount of the ASC ligand, the nucleic acid molecule, the vector, the host cell, or the pharmaceutical composition, according to the invention, or any combination thereof, to a subject in need thereof.

Such methods may comprise an optional first step of preparing the inventive ASC ligand, nucleic acid molecule, vector, host cell, or pharmaceutical composition, prior to administering an effective amount thereof to the subject.

Neurodegenerative Diseases:

The present invention provides ASC ligands for treating or preventing neurodegenerative diseases. Neurodegenerative diseases are typically chronic, progressive disorders characterized by the gradual loss of neurons in discrete areas of the central nervous system (CNS), such as the brain.

Neurodegenerative diseases envisaged to be treated or prevented by the use of the inventive ASC ligands may preferably be characterized and/or accompanied by dementia. “Dementia” is a general term for a decline in mental ability severe enough to interfere with daily life. Dementia may include decline or loss of memory, communication and language, ability to focus and pay attention, reasoning and judgment, visual perception, or a combination thereof. It may be caused by neurodegeneration in a variety of neurodegenerative diseases.

The present inventors discovered that ASC ligands capable of blocking its aggregation during the course of innate immune inflammatory events are useful in preventing or reducing the formation of A-plaques in the brain. Therefore, ASC ligands according to the invention are particularly envisaged for use in treating neurodegenerative diseases that are characterized and/or accompanied by Aβ-related pathology. The term “Aβ-related pathology” refers to the abnormal production, deposition and aggregation of amyloid-β in the brain.

Preferably, the neurodegenerative disease is selected from Alzheimer's Disease, Parkinsons's Disease, Huntington's disease, Multiple System Atrophy, Amyotrophic Lateral Sclerosis, Sinocerebellar ataxia, Frontotemporal Dementia, Frontotemporal Lobar Degeneration, Mild Cognitive Impairment, Parkinson-plus syndromes, Pick disease, Progressive isolated aphasia, Grey-matter degeneration [Alpers], Subacute necrotizing encephalopathy, or Lewy body dementia, with Alzheimer's Disease being particularly preferred.

“Alzheimer's Disease” (“AD”) is a neurodegenerative brain disease that is a major cause of dementia among the elderly. Symptoms of AD may include progressive loss of learning and memory functions, personality changes, neuromuscular changes, seizures and occasionally psychotic behaviour. Alzheimer's disease is characterized by the deposition of amyloid-β plaques in areas of the brain that are critical for memory and other cognitive functions. It is believed that the deposition of amyloid-β plaques, in these critical areas of the brain, interferes with brain functions.

However, the use of ASC ligands according to the invention does not necessarily have to be limited to neurodegenerative diseases characterized by the formation of Aβ-plaques. ASC is an adaptor protein that fulfils a variety of biological functions. Thus, other neurodegenerative diseases are in line for treatment or prevention with the inventive ASC ligands as well.

Further neurodegenerative diseases envisaged for treatment or prevention according to the present invention include hereditary ataxia, congenital nonprogressive ataxia, early-onset cerebellar ataxia, late-onset cerebellar ataxia, cerebellar ataxia with defective DNA repair, hereditary spastic paraplegia, infantile spinal muscular atrophy, type I [Werdnig-Hoffman], inherited spinal muscular atrophy, systemic atrophies primarily affecting the central nervous system, paraneoplastic neuromyopathy and neuropathy, postpolio syndrome, Degenerative diseases of basal ganglia, Hallervorden-Spatz disease, progressive supranuclear ophthalmoplegia [Steele-Richardson-Olszewski], Neurogenic orthostatic hypotension [Shy-Drager], dystonia, tremor, chorea, Restless legs syndrome, Stiff-man syndrome, extrapyramidal and movement disorders, Multiple sclerosis, acute disseminated demyelination, Neuromyelitis optica [Devic], Acute and subacute haemorrhagic leukoencephalitis [Hurst], Periaxial encephalitis, Schilder disease, Central demyelination of corpus callosum, Central pontine myelinolysis, Acute transverse myelitis in demyelinating disease of central nervous system, Subacute necrotizing myelitis, Concentric sclerosis, Epilepsy, Localization-related (focal)(partial) idiopathic epilepsy and epileptic syndromes with seizures of localized onset, Localization-related (focal)(partial) symptomatic epilepsy and epileptic syndromes with simple partial seizures, Localization-related (focal)(partial) symptomatic epilepsy and epileptic syndromes with complex partial seizures, myoclonic epilepsy in infancy, neonatal convulsions (familial), Childhood absence epilepsy [pyknolepsy], absence epilepsy, myoclonic epilepsy [impulsive petit mal], epilepsy with myoclonic absences, myoclonic-astatic seizures, Infantile spasms, Lennox-Gastaut syndrome, Salaam attacks, Symptomatic early myoclonic encephalopathy, West syndrome, Epilepsia partialis continua [Kozhevnikof], Grand mal seizures, Petit mal, Status epilepticus, Grand mal status epilepticus, Petit mal status epilepticus, Complex partial status epilepticus, Migraine, Cluster headache syndrome, Vascular headache, Tension-type headache, Chronic post-traumatic headache, Narcolepsy and cataplexy, Kleine-Levin syndrome

“Treatment” or “treating” include the following goals: (1) preventing undesirable symptoms or pathological states from occurring in a subject who has not yet been diagnosed as having them; (2) inhibiting undesirable symptoms or pathological states, i.e., arresting their development; or (3) ameliorating or relieving undesirable symptoms or pathological states, i.e., causing regression of the undesirable symptoms or pathological states.

The ASC ligand, the nucleic acid molecule, the vector, the host cell, and (pharmaceutical) composition of the invention may be used for human and also for veterinary medical purposes, preferably for human medical purposes. The term “subject”, “patient” or “individual” as used herein thus generally includes humans and non-human animals and preferably mammals (e.g., non-human primates, including marmosets, tamarins, spider monkeys, owl monkeys, vervet monkeys, squirrel monkeys, and baboons, macaques, chimpanzees, orangutans, gorillas; cows; horses; sheep; pigs; chicken; cats; dogs; mice; rat; rabbits; guinea pigs; etc.), including chimeric and transgenic animals and disease models. In the context of the present invention, the term “subject” preferably refers a non-human primate or a human, most preferably a human.

An “effective amount” means an amount of the active agent(s) or composition that is sufficient to elicit a desired biological or medicinal response in a tissue, system, animal or human that is being sought. An “effective amount” is thus preferably sufficient for inducing a positive modification of the disease to be treated, i.e. for alleviation of the symptoms of the disease being treated, reduction of disease progression, or prophylaxis of the symptoms of the disease being prevented. At the same time, however, an “effective amount” is preferably safe, i.e. small enough to avoid serious side-effects, that is to say to permit a sensible relationship between advantage and risk. Typically, an “effective amount” may vary in connection with the particular condition to be treated and also with the age, physical condition, body weight, sex and diet of the patient to be treated, the severity of the condition, the duration of the treatment, the nature of the co-therapy, of the particular pharmaceutically acceptable excipient used, the treatment regimen and similar factors. The “effective amount” may be determined by standard pharmaceutical procedures in cell cultures or experimental animals, e.g., for determining the LD50 (the dose lethal to 50% of the population) and the ED50 (the dose therapeutically effective in 50% of the population). Exemplary animal models suitable for determining an “effective amount” include, without implying any limitation, rabbit, sheep, mouse, rat, dog and non-human primate models. The dose ratio between toxic and therapeutic effects is the therapeutic index and can be expressed as the ratio LD50ED50. Active agents or compositions which exhibit large therapeutic indices are generally preferred. The data obtained from the cell culture assays and animal studies can be used in formulating a range of dosage for use in humans. The dosage of such compounds lies preferably within a range of circulating concentrations that include the ED50 with little or no toxicity. In the context of the present invention, an “effective amount” may range from about 0.001 mg to 10 mg, from about 0.01 mg to 5 mg, from about 0.1 mg to 2 mg per dosage unit or from about 0.01 nmol to 1 mmol per dosage unit, such as from 1 nmol to 1 mmol per dosage unit, or from 1 μmol to 1 mmol per dosage unit. An “effective amount” may also range (per kg body weight) from about 0.01 mg/kg to 10 g/kg, from about 0.05 mg/kg to 5 g/kg, or from about 0.1 mg/kg to 2.5 g/kg.

Administration may be accomplished via subcutaneous, intravenous, intramuscular, intraarterial, intradermal, intraperitoneal, intravascular (i.v.), intranasal, transdermal, intralesional, intratumoral, intracranial, intrapulmonal, intracardial, sublingual, rectal, buccal or vaginal administration routes. Administration may be local or systemic. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant. Systemic administration may be achieved by oral administration or by injection, which may be needle-free injection (jet injection) and/or needle injection.

The ASC ligand, nucleic acid, vector, host cell or (pharmaceutical) composition may be administered to a subject in need thereof several times a day, daily, every other day, weekly, or monthly.

The ASC ligand, nucleic acid, vector, host cell or (pharmaceutical) composition and optionally other active agents described in the section “Pharmaceutical composition” either sequentially (at different times via the same or different administration routes) or simultaneously (at the same time via the same or different administration routes) or in the same pharmaceutical composition. The sequential administration scheme is also referred to as “time-staggered” administration. Time-staggered administration includes regimens where a first dose of ASC ligand, nucleic acid, vector, host cell or (pharmaceutical) composition is administrated e.g. prior, concurrent or subsequent to a second dose of the same ASC ligand, nucleic acid, vector, host cell or (pharmaceutical) composition, or a dose of another active agent (which may be an ASC ligand, nucleic acid, vector, host cell or (pharmaceutical) composition of the invention or another active agent).

Nucleic acids or vectors encoding ASC ligands according to the invention may also be used in gene therapy. “Gene therapy” generally refers to the manipulation of a genome for therapeutic purposes and includes the use of genome-editing technologies for correction of mutations that cause disease, the addition of therapeutic genes to the genome, the removal of deleterious genes or genome sequences, and the modulation of gene expression. Gene therapy may involve in vivo or ex vivo transformation of the subject's cells. For instance, nucleic acids or vectors encoding ASC ligands according to the invention may be administered to a subject suffering from a neurodegenerative disease, where they are expressed to yield the encoded ASC ligand. Typically, nucleic acids may be delivered to the subject in the form of suitable vectors enabling the transfer and expression of the encoded ASC ligand. Such vectors are described elsewhere herein and include, e.g. viral vectors. Alternatively, nucleic acids may be delivered in “naked” form, or be complexed with lipids, polymers or other suitable complexing agents.

### Diagnostic Methods and Kits

The present invention further relates to diagnostic methods exploiting the presence of autoantibodies against ASC aggregates. The diagnostic uses and methods described herein may be conducted in vivo or in vitro using an isolated sample of the subject to be diagnosed.

Accordingly, in a further aspect the present invention relates to apoptosis-associated speck-like protein containing a CARD (ASC) for use in a method of diagnosing a neurodegenerative disease or the risk of developing a neurodegenerative disease in a subject, said method comprising (i) contacting said sample with an ASC protein comprising or consisting of an amino acid sequence corresponding to SEQ ID NO: 1, or a homolog, isoform, variant, fragment, derivative or aggregate thereof, and (iii) detecting the binding of an analyte to said ASC protein, or a homolog, isoform, variant, fragment, derivative or aggregate thereof.

Further, the invention also relates to a method of diagnosing a neurodegenerative disease or the risk of developing a neurodegenerative disease in a subject, said method comprising (i) optionally collecting a sample from a subject who is suspected to be afflicted with or at the risk of developing said disease, (ii) contacting said sample with an ASC protein comprising or consisting of an amino acid sequence corresponding to SEQ ID NO: 1, or a homolog, isoform, variant, fragment, derivative or aggregate thereof, and (iii) detecting the binding an analyte to said ASC protein, or a homolog, isoform, variant, fragment, derivative or aggregate thereof.

The diagnostic uses and methods may involve the provision of said ASC binding protein or its homolog, isoform, variant, fragment, derivative or aggregate on a solid support. Analyte detection may be accomplished using well-known techniques including immunodiffusion, immunoblotting techniques, immunofluorescence, enzyme immunoassays and flow cytometry for multiplex bead-based assays.

The analyte may preferably be an autoantibody. An autoantibody is an antibody which recognized or binds to an antigen of the host producing said antibody. The present inventors suggest that human anti-ASC aggregate antibodies could prevent cross-seeding of amyloid-β peptides in the brain. However, compromised antibody generation and immune surveillance during aging-associated immune senescence may lead to the production of reduced levels of autoantibodies directed against ASC aggregates, and therefore to a higher risk of amyloid-β aggregation. Endogenous anti ASC aggregate antibody titers may thus be used as possible markers of disease progression, in particular during the clinically silent pre-stages of neurodegenerative disease such as Alzheimer's disease.

Accordingly, the diagnostic uses and methods may comprise a further step of quantifying the analyte in the sample and optionally comparing said quantity to a reference.

The reference may be a value such as an antibody titer obtained by subjecting a healthy subject or a sample derived from said healthy subject to the same diagnostic method. The reference may be derived from a subject different from the subject to be diagnosed or may have been derived from the same subject to be diagnosed at an earlier time point. The reference may also be a value such as an antibody titer derived from a plurality of healthy subjects, e.g. a median value.

A reduced quantity of the analyte in the subject to be diagnosed or the sample derived from said subject to be diagnosed as compared to the reference is indicative of a neurodegenerative disease or the risk of risk of developing said disease. The reduced immune surveillance and production of autoantibodies during aging is thought to increase the risk of amyloid-β aggregation.

The diagnostic uses and methods described herein may thus preferably be characterized or accompanied by the presence of ASC aggregation and/or amyloid-β aggregation.

Preferably, said neurodegenerative disease may be selected from Alzheimer's Disease, Parkinsons's Disease, Huntington's disease, Multiple System Atrophy, Amyotrophic Lateral Sclerosis, Sinocerebellar ataxia, Frontotemporal Dementia, Frontotemporal Lobar Degeneration, Mild Cognitive Impairment, Parkinson-plus syndromes, Pick disease, Progressive isolated aphasia, Grey-matter degeneration [Alpers], Subacute necrotizing encephalopathy, or Lewy body dementia, with Alzheimer's Disease being particularly preferred.

The present invention further provides a diagnostic kit for carrying out the diagnostic methods and uses described herein, comprising an ASC protein or ASC aggregate and detection means for detecting the binding of autoantibodies to said protein or aggregate.

### Combination Therapy

The inventive ASC ligand, nucleic acid molecule, vector, host cell, or pharmaceutical composition also be used in combination therapy. To that end, any therapeutic or prophylactic means useful for treating or preventing the neurodegenerative diseases may be used in combination with the treatment according to the present invention.

Thus, a subject afflicted by a neurodegenerative disease may be treated with the inventive ASC ligand, nucleic acid molecule, vector, host cell, or pharmaceutical composition, and additionally receive one or more of the following compounds or active agents: cholinesterase inhibitors such as donepezil, galantamine, rivastigmine or tacrine; MDA (N-methyl-D-aspartate) receptor antagonists such as memantine; vitamin E; vitamin A; alpha-tocopherol; selenium; zinc; folic acid; vitamin 812; omega-3 fatty acids; docosahexaenoic acid (DHA); or combinations thereof. These compounds and active agents may be administered simultaneously or sequentially in a time-staggered administration scheme as compared to the inventive ASC ligand, nucleic acid molecule, vector, host cell, or pharmaceutical composition.

### In Vitro Methods

In further aspects, the present invention also provides an in vitro method for determining if a candidate ligand is capable of interacting with, preferably binding to, an ASC protein comprising or consisting of an amino acid sequence corresponding to SEQ ID NO: 1, or a homolog, isoform, variant, fragment or derivative thereof, comprising: (i) contacting the candidate ligand with an ASC protein comprising or consisting of an amino acid sequence corresponding to SEQ ID NO: 1, or a homolog, isoform, variant, fragment or derivative thereof; and (ii) detecting the binding of the candidate ligand.

The method may further comprise a step of evaluating, whether the candidate ligand inhibits a) ASC aggregation and/or b) amyloid-β aggregation in vitro. This additional method step may be accomplished using the methods described in the appended examples.

In further aspects, the present invention provides an in vitroscreening method for ASC ligands, said method comprising the steps of: (a) providing an ASC protein comprising or consisting of an amino acid sequence corresponding to SEQ ID NO: 1, or a homolog, isoform, variant, fragment or derivative thereof, (b) contacting said ASC protein with a candidate ligand; and (c) detecting the specific binding of said candidate ligand to said ASC protein.

The invention further relates to ASC ligands obtainable by said method, said ASC ligand being selected from an antibody, a protein, a peptide, a nucleic acid or a small molecule organic compound.

In a further aspect, the present invention relates to in vitro methods for determining the presence of ASC aggregates in a sample, comprising the steps of: i) contacting a sample obtained from a subject with an ASC ligand as described herein, and ii) detecting the specific binding of said ASC ligand; wherein detectable binding of said ASC ligand is indicative of the presence of ASC aggregates in the subject.

The sample may for instance be a brain biopsy. The detectable binding of said ASC ligand may be indicative of a neurodegenerative disease or the risk of developing the same, which is characterized or accompanied by the presence of ASC aggregation and/or amyloid-β aggregation. Said neurodegenerative disease may be selected from any of the neurodegenerative diseases described herein, and may preferably be Alzheimer's Disease.

### FIGURES

In the following a brief description of the appended figures will be given. The figures are intended to illustrate the present invention in more detail. However, they are not intended to limit the subject matter of the invention in any way.

FIG. 1 Microglia released ASC specks bind to and cross-seed β-amyloid peptides (a) ASC specks detected in (Con-in, AD-in) and outside (Con-ex, AD-ex) of microglia in hippocampal sections of AD brains and age-matched non-demented controls (Con), (n=10 biologically independent human cases, mean±SEM, one-way ANOVA, Tukey test, ***p<0.0001) (b) Microglia containing ASC specks and free ASC specks in the hippocampus of APP/PS1 mice and quantification at various ages (n=5 biologically independent animals, mean±SEM, one-way ANOVA, Tukey test, ***p=0.0002) (a,b: Bar=10 μm). (c) Flow cytometry of Alexa-488-labelled-ASC specks released by LPS-primed murine microglia upon exposure to nigericin (10 μM) or ATP (5 mM) (d) Quantification of ASC-Alexa 488+ specks per pI of cell-free supernatants of resting or activated microglia (n=3 technical replicates, mean±SD) and confocal imaging of LPS-primed, ATP-activated microglia including a magnification of an extracellular ASC speck (Bar=6 μm). (e) Extracellular TAMRA-Aβ₁₋₄₂-binding to THP-1-cells released GFP-linked ASC specks. (f) Experimental schematic of Aβ binding experiments employing ASC release by immunostimulated primary microglia. (g) Flow cytometry analysis of supernatants derived from wt or ASC^{-/-} and control (Con) and immunostimulated (activated) primary microglia in the presence and absence of Aβ₁₋₄₂ and (h) quantification of the number of ASC/Aβ₁₋₄₂ events in wt and ASC^{-/-} supernatants (n=4 biologically independent experiments, mean±SEM, one-way ANOVA, Tukey test, ***p<0.0001). (i) Thioflavin-T fluorescence assay of ASC speck and Aβ₁₋₄₂ co-incubation (n=2 biologically independent samples, mean±SEM) (j) Confirmation of ASC speck-enhanced Aβ₁₋₄₂ oligomer formation by immunoblot detection and (k) quantification at single time points (n=4 biologically independent experiments, mean±SEM, two-tailed Student's t-test, 2 h **p=0.0089, 4 h **p=0.0093, 6 h ***p=0.001, 24 h ***p<0.0001). Experiments shown in d, i, j were independently replicated twice.

FIG. 2 ASC specks cosediment with Aβ and form the core of murine and human Aβ plaques. (a) Schematic of ASC speck-Aβ co-sedimentation experiments at 0 and 6 h. (b) Supernatants (sup) and pellets (pellet) of in vitro incubations of either (1) Aβ₁₋₄₂ or Aβ₁₋₄₀ alone, (2) Aβ₁₋₄₂ or Aβ₁₋₄₀ together with ASC specks or (3) ASC specks alone. Densitometry of ASC speck levels at 0 and 6 h after coincubation with either Aβ₁₋₄₀ or Aβ₁₋₄₂ given as percentage (n=4 biologically independent samples, mean±SEM) (c) Co-immunoprecipitation experiments demonstrating Aβ to ASC specks binding using A-antibody 82E1 for detection in wild-type (wt) and APP/PS1 (tg) brain homogenates and (d) quantification at 3, 8, and 12 months of age (n=4 biologically independent animals, mean±SEM, one-way ANOVA, Tukey test, ***p<0.0001). (e) Dot blot analysis of the fluffy fiber and core compartment of murine Aβ deposits by sucrose gradient centrifugation from APP/PS1 (tg) and wild-type (wt) mice at 8 months. (f) Co-immunohistochemistry of an early Aβ deposit in an APP/PS1 mouse at 4 months (4 m) using ASC (AL177) and Aβ (6E10) antibodies (bar=10 μm). (g) Immunoprecipitation experiments detecting ASC bound Aβ in human brain homogenates from age-matched, non-demented controls (Con) and Alzheimer patients (AD) and (h) quantification (n=27 biologically independent human cases, mean±SEM, two-tailed Student's t-test, ***p<0.0001). (i) Dot blot analysis of the fluffy fiber and core compartment of Aβ deposits from age-matched non-demented controls (Con), patients suffering from mild cognitive impairment due to AD (MCI) and AD by sucrose gradient centrifugation. (j) Co-immunohistochemistry of a deposit in the hippocampus of an AD patient using ASC (AL177) and Aβ (6E10) antibodies. Arrows indicate AL177 (red) and 6E10 (green) immunopositivity (bar=10 μm). Experiments shown in c, e, g and h have been independently replicated 3 times. Experiments shown in f, h have been independently replicated 5 times.

FIG. 3 ASC knockout reduced Aβ pathology and spatial memory deficits in APP/PS1 mice. APP/PS1/ASC^{-/-} mice and respective controls were analyzed for Aβ load and spatial memory dysfunction. (a) Representative micrographs of hippocampi (Bar=500 μm) stained for Aβ using antibody 6E10. (b) Total Aβ immunostained area and number of Aβ-immunopositive deposits (n=8 biologically independent animals, mean±SEM, two-tailed Student's t-test, ***p<0.0001). (c) Spatial memory was assessed in the Morris water maze (mean±SEM). Time needed to reach the hidden platform (latency) in wt, ASC^{-/-}, APP/PS1, and APP/PS1/ASC^{-/-} mice. (d) Integrated time travelled (AUC=area under the curve) (mean of n=12 for wt, n=19 for ASC^{-/-}, n=14 for APP/PS1, and n=21 for APP/PS1/ASC^{-/-} biologically independent animals ±SEM; one-way ANOVA, Tukey test, APP/PS1 vs APP/PS1/ASC^{-/-}: ***p=0.0009, other: ***p<0.0001). (e) Spatial probe trial day 9, where platform was removed and time spent in quadrants was recorded. Q1: platform location at day 1-8. The values for time spent in all other quadrants were averaged (o.a.) (mean of n=12 for wt, n=19 for ASC^{-/-}, n=14 for APP/PS1, and n=21 for APP/PS1/ASC^{-/-} biologically independent animals ±SEM; one way ANOVA, Tukey test, APP/PS1 ***p=0.0002, APP/PS1/ASC^{-/-}*p=0.0236). (f) Representative runs of single mice. (g) APP/PS1 and APP/PS1/ASC^{-/-} mice received bilateral hippocampal injections with lysates (lys) from either APP/PS1 or wt mice at 3 months of age. Brains were analyzed at 8 months. Representative micrographs of injected hippocampi (Bar=500 μm). APP/PS1 brain lysate injection increased Aβ pathology compared to wt brain lysate in APP/PS1 mice (APP/PS1-lys vs wt-lys), but not in APP/PS1/ASC^{-/-} animals as detected for (h) total Aβ immunostained area (total area) or number of Aβ deposits (n=8 biologically independent samples, mean±SEM, one-way ANOVA, Tukey test, *p=0.0252, ***p<0.0001). (i) ELISA analysis of dissected hippocampi from an independent group of animals confirmed the histological evaluation (n=10 biologically independent samples, mean±SEM, one-way ANOVA, Tukey test, ***p<0.0001). (j) Levels of amyloid precursor protein (APP) and c-terminal cleavage fragments (α-CTF, β-CTF) remained unchanged. Non-injected APP/PS1 or APP/PS1/ASC^{-/-} brains (non-inj.) as well as wt brains served as a control, (k) densitometric quantification (n=8 biologically independent samples, mean±SEM, one-way ANOVA, Tukey test, ***p<0.0001). Recombinant Aβ₁₋₄₂ peptide served as a positive control (Pos. con). Experiments shown in a, g were independently replicated twice, experiments shown in j were independently replicated 4 times.

FIG. 4 Reduced spreading of Aβ pathology by ASC deficient APP/PS1 brain lysate or anti-ASC antibody co-injection. APP/PS1 mice received bilateral intrahippocampal injections of brain lysates either derived from APP/PS1 or APP/PS1/ASC^{-/-} animals at 3 months. As deposition was quantified at 8 months by Aβ immunostaining using antibody 6E10. (a) Representative micrographs of injected hippocampi (Bar=500 μm). (b) Total Aβ immunostained area and number of Aβ plaques (n=5 biologically independent samples, mean±SEM, one-way ANOVA Tukey test, total area: *p=0.0105, ***p=0.0003, number of Aβ deposits: APP/PS1 vs APP/PS1/ASC^{-/-} **p=0.0011, APP/PS1 vs non-inj. **p=0.0081). (c) Brain lysates were immunoblotted for APP, α- and β C-terminal fragments (α-CTF and β-CTF), total Aβ and quantified for (d) cerebral Aβ monomer and oligomer (>20 kDa) levels (n=5 biologically independent samples, mean±SEM, one-way ANOVA, Tukey test, monomer: *p=0.0497, **p=0.0097, oligomer: APP/PS1 vs APP/PS1/ASC^{-/-}***p=0.0001, APP/PS1 vs non-inj. p=0.0008). (e) Thioflavin-T (ThT) assays of Aβ₁₋₄₀ co-incubation with ASC specks and increasing concentrations of anti-ASC speck antibody or isotype-specific IgG (iso-IgG) controls (IgG1/2). (f) APP/PS1 mice injected bilaterally with APP/PS1 brain lysate with anti-ASC speck antibody or iso-IgG. Representative micrographs of hippocampi injected with iso-IgG or anti-ASC speck antibody (Bar=500 μm). (h) Total Aβ immunostained area and number of Aβ plaques (n=5 biologically independent samples, mean±SEM, one-way ANOVA Tukey test, **p=0.0058, ***p<0.0001). (g) Brain lysates were immunoblotted as described in (c) and quantified for (i) cerebral Aβ monomer and oligomer (>20 kDa) levels (n=5 biologically independent samples, mean±SEM, one-way ANOVA, Tukey test, monomer: APP/PS1+Iso-IgG vs APP/PS1+anti-ASC ***p=0.0002, APP/PS1+Iso-IgG vs non-inj. ***p<0.0001, oligomer: *p=0.0418, **p=0.0053). Experiments shown in a, f were performed twice, experiments shown in c, g were independently replicated 4 times, Experiments shown in e were independently replicated 3 times.

FIG. 5 Characteristics of microglial ASC speck formation in mice and men. Immunohistochemistry for the microglial marker CD11b and ASC in sections derived from (a) brains of Alzheimer patients (AD) or (b) non-demented controls (Con) omitting either 1^{st} or 2^{nd} antibodies (Bar=15 μm). (c) Percentage of ASC specks detected by immunohistochemistry in- and outside of microglial cells in sections derived from AD patient brains (AD-in, AD-ex) and non-demented, age-matched controls (Con-in, Con-ex) (n=10 biologically independent human cases, mean±SEM, one-way ANOVA, Tukey test, ***p<0.0001) or hippocampus of (d) APP/PS1 mice at the indicated ages given in months (m) (n=10 biologically independent animals, mean±SEM, two-tailed Student's t-test, ***p<0.0001). (e) Number of ASC specks bound to Aβ deposits/visual field observed (n=5 biologically independent animals, mean±SEM, two-tailed Student's t-test, *p=0.0216) (f) ASC expression in brain lysates derived from wild type (WT) and APP/PS1 transgenic mice at 4, 8, 12 and 24 months of age (g) Hippocampal sections of 8 months old wild type (wt), ASC^{-/-}, APP/PS1 and APP/PS1/ASC^{-/-} mice were stained for the microglial marker CD11b and ASC in the presence of the 1^{st} and 2^{nd} antibodies (left panel) or in the absence of the respective 1^{st} antibody (right panel), (Bar=15 μm). (h) Hippocampal sections of 8 month old wt, ASC^{-/-}, APP/PS1 and APP/PS1/ASC^{-/-} mice were stained for the microglial marker CD11b and ASC in the presence of the 1^{st} and 2^{nd} antibodies (left panel) or in the absence of the respective 2^{nd} antibody (right panel), (Bar=15 μm). Experiments shown in a, b, g, h have been independently replicated three times, experiments shown c, d, e have been performed once. Experiments depicted in f have been independently replicated twice.

FIG. 6 Experimental ASC speck formation in primary murine microglia and human THP1 cells. (a) Flow cytometry analysis of conditioned media from primary murine microglia using 2 and 6 μm fluorescent beads for gating ASC specks. (b) Confocal imaging of primary murine microglia exposed to either control solvent (Con), LPS alone, or LPS followed by nigericin (LPS+Nig), or ATP (LPS+ATP). Cells were stained with anti-ASC antibody followed by an A488 conjugate. Arrows show extracellular ASC specks (Bars=24 μm; insets are 4× (bottom left) and 8× (bottom right) magnifications of the areas shown in the squares) (c) Gating strategy for the detection of ASC specks in cell-free supernatants of untreated (−) or LPS-primed, nigericin-activated (10 μM) (LPS+Nig) ASC-mCerulean-expressing THP-1 cells. (d) Confocal imaging of LPS-primed, nigericin-treated THP-1s showing green fluorescent ASC specks in the extracellular space (Bars=38 μm, 8 μm). (e) Quantification of extracellular specks in cell-free supernatants from (n=3 technical replicates, mean±SD), representative of 2 independent experiments. Images of THP-1 cells (in the absence of TAMRA-Aβ₁₋₄₂, (g) showing TAMRA-Aβ surface binding and early incorporation, (h) subsequent upregulation of ASC (green), (i) early ASC speck formation in a cell, which has incorporated TAMRA-Aβ₁₋₄₂ and (j) ASC specks formed within a cell. Experiments shown in a, b, c and f-j have been been independently replicated three times, experiments shown d, e have been performed twice.

FIG. 7 Qualtitative and quantitative description of Aβ-ASC binding. (a) Experimental design and timeline: 3 h LPS and 1 h Nigericin induces a highly inflammatory form of programmed cell death (pyroptosis) causing ASC speck release. Supernatants containing ASC specks were subsequently incubated with Aβ₁₋₄₂ for 6 hand thereafter analyzed by flow cytometry. (b) Upper panel: Immunoprecipitation and immunoblot detection of ASC in unstimulated, immunoactivated wildtype (wt) and ASC knockout (ASC^{-/-}) macrophages. ASC monomer detection is restricted to supernatants of immunoactivated, ASC-competent wt cells and absent in unstimulated wt cells or ASC macrophages. Lower panel: Immunoprecipitation of ASC followed by immunoblot detection of Aβ under the same experimental conditions as described for the upper panel. ASC bound Aβ is exclusively detected in supernatants derived from immunoactivated wt macrophages but not from unstimulated wt or ASC^{-/-} cells. (c) Upper panel: Immunoprecipitation of ASC and immunoblot detection of ASC in unstimulated, immunoactivated wt and ASC^{-/-} microglia. ASC monomer detection is restricted to supernatants of immunoactivated ASC competent wt cells and absent in unstimulated wt cells or ASC^{-/-} macrophages. Lower panel: Immunoprecipitation of ASC followed by immunoblot detection of Aβ under the same experimental conditions as described for the upper panel. ASC bound Aβ is exclusively detected in supernatants derived from immunoactivated wt microglia but not from unstimulated wt or ASC^{-/-} cells. (d) Gating strategy and control group: Gated on debris to exclude remaining cells and larger particles. Recombinant ASC labeled with CFP and Aβ₁₋₄₂ labeled with TAMRA signal in independent quadrants Q1 and Q3. When incubated together, the molecules accumulate and signal in Q2. (n=3 biologically independent samples, mean±SEM, one-way ANOVA, Tukey test, ***p<0.0001). (e) Experimental groups: ASC-mCerulean-expressing, immortalized macrophages show similar results. ASC^{-/-} macrophages show no ASC speck formation and no Aβ₁₋₄₂ accumulation (n=3 biologically independent samples, mean±SEM, one-way ANOVA, Tukey test, activ.+Aβ₁₋₄₂ vs Con ***p=0.0002, activ.+Aβ₁₋₄₂ vs Con+Aβ₁₋₄₂ ***p=0.0009, activ.+Aβ₁₋₄₂ vs activ. ***p=0.0002). (Flow cytometry quantification of ASC-TAMRA-labelled Aβ₁₋₄₀ after immunostimulation of microglia. Experimental design and timeline: 3 h LPS and 1 h ATP induce a highly inflammatory form of programmed cell death (pyroptosis) by which ASC specks are released. Supernatants containing ASC specks were subsequently incubated with Aβ1-40 for 3 h and thereafter analysed by FACS. Experiments depicted in b have been independently replicated three times.

FIG. 8 An immunoprecipitation and enzymatic cleavage-based method for the generation of highly pure ASC specks. (a) Schematic of ASC speck formation upon inflammasome assembly and purification of ASC specks via immunoprecipitation and enzymatic cleavage. Immortalized, ASC-deficient macrophages were transduced with a construct containing ASC-mCerulean with a Flag-tag, and a precision site for the Tobacco Etch Virus protease (TEV) between ASC and mCerulean. Inflammasome activation in these cells, results in ASC aggregation and formation of an ASC speck. The ASC speck-containing the mCerulean and the Flag-tag can be immunopurified, followed by proteolytic cleavage of the mCerulean-Flag-tag by the TEV protease to generate pure ASC specks. (b) Immunoblotting analysis of ASC specks isolated from ASC-mCerulean-Flag macrophages before (−), or after immunoprecipitation using anti-GFP antibodies followed by enzymatic cleavage of the TEV protease. (c) Confocal imaging following immunostaining of ASC and GFP in untreated vs IP+TEV treated ASC specks (bar=3.8 μm (top row), 6.3 μm (middle row), 9 μm (bottom row)). (d) Flow cytometry analysis of anti-ASC-Alexa Fluor 488 and anti-GFP-Alexa Fluor 647 double-stained ASC specks isolated from ASC-mCerulean-Flag macrophages. Anti-mouse IgGs conjugated to Alexa Fluor 488 or 647 were used as controls (bottom panels). Experiments depicted in b-d have been independently replicated four times.

FIG. 9 ASC specks increase the propensity of Aβ peptides to aggregate in a time- and concentration-dependent manner. (a) Thioflavin-T fluorescence assay of ASC specks and Aβ₁₋₄₀ co-incubation showing cross-seeding potency of ASC specks in a time-dependent manner. (b) Western blot detection of time dependent, ASC speck-induced aggregation of Aβ₁₋₄₀. Co-incubation of Aβ₁₋₄₀ with ASC specks increases the propensity to aggregate and increased the formation of high molecular weight Aβ oligomers and protofibrils. (c) Quantification at the indicated time points (n=4 biologically independent samples, mean±SEM, two-tailed Student's t-test, 6 h: ***p=0.0002, 4 and 24 h ***p<0.0001), (d) Western blot analysis of Aβ₁₋₄₂ coincubated with increasing concentrations of ASC specks (0.0-1.75 μM) at 0 and 24 h. (e) Western blot analysis of Aβ₁₋₄₀ coincubated with increasing concentrations of ASC specks (0.0-1.75 μM) at 0 and 24 hr. For both Aβ peptides, co-incubation with ASC specks increased the propensity to aggregate and increased the formation of high molecular oligomers. Note that for Aβ₁₋₄₂ the increase in oligomer formation is paralleled by a reduction of the Aβ monomer and dimer levels. (f) Electron microscopy of Aβ₁₋₄₂, ASC and ASC-Aβ₁₋₄₂ aggregation after 96 h of incubation (Bar=200 nm). (g) Confirmation of ASC speck-enhanced Aβ₁₋₄₀ and Aβ₁₋₄₀ aggregation by turbidity assay (n=3 biologically independent samples, mean±SEM). (h) Thioflavin-T fluorescence assay of ASC specks and Aβ₁₋₄₂ co-incubation showing no cross-seeding potential of ASC specks for the reversed peptide. (i) Thioflavin-T fluorescence assay of Aβ₁₋₄₀ co-incubation with ASC specks and two different concentrations with bovine serum albumin. While ASC specks cross-seed Aβ₁₋₄₀ in a time-dependent manner, neither 0.22 μM nor 0.66 μM BSA affected Aβ₁₋₄₀ aggregation. Experiments depicted in a, b, d, and e were independently replicated four times, experiments shown in f, h, i were independently replicated three times.

The publication by Franklin et al. (Nature Immunology, 2014, 15 (8): 727-737) describes ASC and its extracellular and prionoid activities propagating inflammation. Therein, the authors describe ASC antibodies opsonizing ASC specks and thereby increasing inflammation. However, inflammation resulting therefrom is secondary to the etiology of Alzheimer's disease. Rather, the present finding is crucial to the etiology of Alzheimer, e.g. inflammatory events triggered by Aβ aggregation. The in vivo experiments of FIG. 9 show that such Aβ aggregation is triggered by ASC specks.

FIG. 10 The ASC PYD domain is critical for Aβ cross-seeding. (a) Immunoblots were probed for Aβ using antibody 82E1 revealing time-dependent aggregation of Aβ₁₋₄₀. Co-incubation of Aβ₁₋₄₀ with recombinant ASC specks (recASC) promotes aggregation and increases the formation of high molecular weight Aβ oligomers. Notably, formation of intermediate Aβ oligomers (from 28-62 kDa bands) is observed and increased with incubation time. (b) Immunoblot for ASC revealing time-dependent auto-aggregation. (c) Immunoblots were probed for Aβ using antibody 82E1 revealing time-dependent aggregation of Aβ₁₋₄₂. Co-incubation of Aβ₁₋₄₂ with recombinant ASC specks (recASC) promotes aggregation and increases the formation of high molecular weight Aβ oligomers. Notably, formation of intermediate Aβ oligomers (from 28-62 kDa bands) is observed and increased with incubation time. (d) Immunoblots were probed for ASC revealing time-dependent auto-aggregation. (e) Recombinant mutant ASC was generated by introducing point mutations at residues K21A, K22A and K26A in the ASC-PYD domain. Purified, recombinant, mutant ASC specks were used for the Aβ aggregation assay. Immunoblots were probed for A using antibody 82E1 revealing time-dependent aggregation of Aβ. Co-incubation of recombinant mutant ASC specks (recASC; K21A, K22A and K26A) failed to increase high molecular weight Aβ oligomer levels. No intermediate Aβ oligomers (from 28-62 kDa bands) are seen in Aβ supplemented with recombinant mutant ASC specks. (f) Immunoblots were stained for ASC revealing no auto-aggregation of recombinant mutant ASC specks. (g) Purified recombinant mutant ASC generated by introducing point mutations at residues D134R and Y187E in the ASC-CARD domain were used for the Aβ aggregation assay. Immunoblot was probed for Aβ using antibody 82E1 revealing time-dependent aggregation of Aβ₁₋₄₀. Increased levels of high molecular weight Aβ oligomers are evident after 2 hours of incubation in Aβ samples upon addition of recombinant mutant ASC (recASC; D134R and Y187E) specks. The levels of Aβ oligomers increased with incubation time. Formation of intermediate Aβ oligomers (from 28-62 kDa bands) is also apparent and increased with incubation time. (h) Immunoblot stained for ASC revealing auto-aggregation of recombinant ASC-CARD mutant ASC (D134R and Y187E) specks. (i) Quantification at the indicated time points (n=3 biologically independent samples, mean±SEM, two-tailed Student's t-test, 2 h: **p=0.0012, 4 h: **p=0.0052, 6 h: **p=0.0032, 12 h: **p=0.0033, 48 h: ***p=0.0003, 24 and 72 h: ***p<0.0001). (j) Quantification at the indicated time points (n=3 biologically independent samples, mean±SEM, two-tailed Student's t-test, 2 h: *p=0.0212, 4 h: **p=0.0012, 6 h: *p=0.0240, 12 h: **p=0.0018, 24 h: **p=0.0069, 48 h **p=0.0031, 72 h: ***p=0.0002). (k) Ribbon diagrams displaying the positions of the respective mutations in the PYD- and CARD-domains of ASC. Experiments depicted in a-h have been independently replicated three times.

FIG. 11 Thioflavin t fluorescence scans and concomitant cosedimentation assay of Aβ peptides and ASC specks. Thioflavin t (ThT) fluorescence spectra of (a) supernatant and (b) pellet fractions of Aβ₁₋₄₀ (Aβ₁₋₄₀ alone or in combination with ASC (Aβ₁₋₄₀+ASC)) at 0 and 6 h post incubation monitored at λem between 460 and 605 nm with excitation at 446 nm (mean±SEM). Excitation and Emission slit was set at 10 nm. (c) Quantification of the maximal emission values (485 nm) and statistical analysis (n=3 biologically independent samples ±SEM, two-tailed Student's t-test, **p=0.0011, p=***0.0003). ThT fluorescence spectra of (d) supernatant and (e) pellet fractions of Aβ₁₋₄₂ (Aβ₁₋₄₂ alone or in combination with ASC (Aβ₁₋₄₂+ASC)) at 0 and 6 hours post incubation obtained under the identical conditions as above (f) Quantification of the λua max values (485 nm) and statistical analysis (n=3 biologically independent samples ±SEM, two-tailed Student's t-test, **p=0.0023, ***p<0.0001). (g) Aβ₁₋₄₀ or (h) Aβ₁₋₄₂ in the presence or absence of ASC specks with anti-Aβ antibody (82E1) (1=A alone, 2=Aβ+ASC, 3=ASC). Experiments depicted in g and h were independently replicated three times.

FIG. 12 ASC immunopositivity is found in the centre of Aβ deposits of APP/PS1 mice and AD patients. (a) The identical samples from mouse fiber or core preparations as analyzed in FIG. 3 were probed only with the secondary antibody used for ASC detection. (b) The identical samples from human fiber or core preparations as analyzed in FIG. 2 were probed only with the secondary antibody used for ASC detection. (c) Recombinant ASC and synthetic Aβ₁₋₄₂ were sequentially diluted and immunoprobed using ASC (AL177) or Aβ (6E10) antibodies. Further methodological reading^{32xx31} (d) Immunostaining for Aβ (6E10) and ASC (AL177) in sections derived from APP/PS1 mice with and without 1^{st} and 2^{nd} antibodies (bar=15 μm). (e) Control section from ASC^{-/-} animals stained for Aβ or ASC (bar=20 μm). Immunoprecipitation of ASC followed by immunoblot detection of ASC (f) or immunoblot detection of Aβ (g) in brain samples from non-demented controls (Con) and AD patients (AD). A further control shows the same detection of in vitro coincubation of Aβ₁₋₄₂, ASC and Aβ₁₋₄₂+ASC. (h) Immunostaining for Aβ (green) and ASC (red) in sections derived from AD brains (AD) and age-matched, non-demented controls (Con), and omission of both 1^{st} antibodies as a negative control (bar=15 μm). Immunoprecipitation experiment showing (i) immunoprecipitation of ASC followed by Western blot detection of ASC in brain samples from patients suffering from vascular dementia (VD), frontotemporal dementia (FTD), corticobasal degeneration (CBD) and Alzheimer's disease (AD). (j) Immunoprecipitation of ASC followed by Western blot detection of Aβ in the same brain samples. ASC-bound Aβ was only detected in AD patients. Experiments depicted in a-c and f, g, i, have been independently replicated three times. Experiments depicted in d, e, h have been independently replicated five times.

FIG. 13 Aβ levels and spatial navigation memory in APP/PS1/ASC^{-/-} mice at 8 and 12 month of age. (a) ELISA quantification from SDS and FA fractions for Aβ₁₋₃₈, Aβ₁₋₄₀ and Aβ₁₋₄₂ from 8-month old APP/PS1 and APP/PS1/ASC^{-/-} mice (n=5 biologically independent animals ±SEM; two-tailed Student's t-test, SDS: Aβ₁₋₃₈ **p=0.0016, Aβ₁₋₄₀ **p=0.0025, Aβ₁₋₄₂ ***p=0.0008, FA: Aβ₁₋₃₈ **p=0.0021, Aβ₁₋₄₀ **=0.0040. Aβ₁₋₄₂ *p=0.0106). (b) Spatial memory was assessed in the Morris water maze. Distance travelled to platform in wt, ASC^{-/-}, APP/PS1 and APP/PS1/ASC mice (mean±SEM). Quantification was performed by integrating distance travelled (area under the curve) (n=12 for wt, n=19 for ASC^{-/-}, n=14 for APP/PS1, and n=21 for APP/PS1/ASC^{-/-} biologically independent animals, mean±SEM; one-way ANOVA, Tukey test, APP/PS1 vs APP/PS1/ASC^{-/-} ***p=0.0005, other: ***p<0.0001). (c) ELISA quantification from SDS and FA fractions for Aβ₁₋₃₈, Aβ₁₋₄₀ and Aβ₁₋₄₂ from 12-month old APP/PS1 and APP/PS1/ASC^{-/-} mice (n=5 biologically independent animals, mean±SEM; two-tailed Student's t-test, SDS: Aβ₁₋₃₈ ***p<0.0001, Aβ₁₋₄₀ **p=0.0015, Aβ₁₋₄₂ ***p=0.0002, FA: Aβ₁₋₃₈ ***p=0.0009, Aβ₁₋₄₀ **p=0.0084, Aβ₁₋₄₂ **p=0.0010). (d) Hippocampal sections from wt, ASC^{-/-}, APP/PS1 and APP/PS1/ASC^{-/-} animals at 12 months of age (bar=500 μm) and quantification of total area and the number of Aβ deposits (n=6 biologically independent animals, mean±SEM, two-tailed Student's t-test, ***p<0.0001). Spatial memory was assessed by Morris water maze testing. (e) Time needed to reach the platform (latency) in wild type (wt), ASC^{-/-}, APP/PS1, and APP/PS1/ASC^{-/-} mice (mean±SEM) and integrated time travelled (AUC=area under the curve) (n=11 for wt, n=11 for ASC^{-/-}, n=17 for APP/PS1, and n=15 for APP/PS1/ASC^{-/-} biologically independent animals, mean±SEM; one-way ANOVA, Tukey test, wt vs APP/PS1 ***p<0.0001, ASC^{-/-} vs APP/PS1 ***p=0.0003, APP/PS1 vs APP/PS1/ASC^{-/-}**p=0.0022). (f) Distance travelled to platform (Distance to platform) in wt, ASC^{-/-}, APP/PS1, and APP/PS1/ASC^{-/-} mice (mean±SEM) and integrated distance travelled (n=11 for wt, n=11 for ASC^{-/-}, n=17 for APP/PS1, and n=15 for APP/PS1/ASC^{-/-} biologically independent animals, mean±SEM; one-way ANOVA, Tukey test, APP/PS1 vs APP/PS1/ASC^{-/-} ***p=0.0004, other: ***p<0.0001). At day 9, 24 h after the last training session, a spatial probe trial was conducted, where the platform was removed and the time animals spent in the quadrants was recorded. (g) Q1: platform location at day 1-8. The values for the time spent in all other quadrants were averaged (o.a.) (n=12 for wt, n=19 for ASC^{-/-}, n=14 for APP/PS1, and n=21 for APP/PS1/ASC^{-/-} biologically independent animals, mean±SEM; one-way ANOVA, Tukey test, ASC^{-/-}*p=0.0329). (h) Representative runs of a single mouse are depicted. Experiments shown in d were independently replicated twice.

FIG. 14 Age-dependent modulation of cortical Aβ levels by ASC in APP/PS1 mice and analysis of caspase-1 cleavage, NEP AND IDE. (a) Immunohistochemistry of cortical sections from wt, ASC^{-/-}, APP/PS1 and APP/PS1/ASC^{-/-} animals at 3, 8 and 12 months of age using antibody 6E10 (bar=500 μm). (b) Quantification of Aβ deposition given as total Aβ covered area (total area) and as number of Aβ deposits in the respective cortical section of APP/PS1 and APP/PS1/ASC^{-/-} mice at 8 (n=3 biologically independent animals, mean±SEM, two-tailed Student's t-test, number of Aβ deposits ***p=0.0009, total area ***p<0.0001) and 12 months of age (n=6 biologically independent animals, mean±SEM, two-tailed Student's t-test, number of Aβ deposits ***p=0.0003, total area ***p=0.0002). (c-f) Analysis of experiment I-IV for caspase-1, neprilysin and insulin-degrading enzyme levels in animals undergoing the respective experimental protocol (see also Extended Data FIG. 11b,c: EXPI). Detection of β-actin levels served as a loading control. Positive controls represent wt mouse brain lysate spiked with caspase-1, NEP, and IDE. (R=right hemisphere lysate, L=left hemisphere lysate). Given are the genetic background of the injected animals (EXPI: APP/PS1 mice; EXPII: APP/PS1, APP/PS1/ASC^{-/-} mice; EXPIII: APP/PS1 mice; EXP IV: APP/PS1 mice) and respective controls as well as the injected material/brain lysate. Experiments shown in a were independently replicated twice. Experiments shown in c-f were independently replicated three times.

FIG. 15 Microglial Aβ phagocytosis in 8 month old APP/PS1 and APP/PS1/ASC^{-/-} mice and experimental schematics of Aβ in vivo seeding experiments (a) Representative scatter plots of mice analyzed for microglial amyloid content after intraperitoneal (i.p) injection of methoxy-XO4 (MxO4) and isolation of microglia at 8 months of age and quantification of amyloid content revealing no differences between groups (n=11 for APP/PS1, n=10 for APP/PS1/ASC^{-/-} biologically independent animals, mean±SEM, two-tailed Student's t-test). (b) Design of in vivo experiments EXP I-IV showing the genetic background of host mice and injected materials. (c) Time schedule of Exp I. (d) Time schedule of experiments II-IV. (e) Brain lysates were generated as described by Fritschi et al. Acta Neuropathol. 2014 October; 128(4):477-84 and also Meyer-Luehmann et al. Science. 2006 Sep. 22; 313(5794):1781-4. Scheme of the preparation of the injection material from mouse forebrain. Aliquots of brain homogenates from APP/PS1 and APP/PS1//ASC^{-/-} mice were analyzed for Aβ content by immunoblot using antibody 82E1 and anti-actin antibody to normalize for protein loading. (f) Site of bi-hippocampal injection and sections analyzed with an equal distance of 120 μm to each other. Experiments shown in a, e were independently replicated twice.

FIG. 16 ASC specks cause rostro-caudal spreading of Aβ pathology in APP/PS1 mice without affecting microglial phagocytosis. (a) Representative micrographs of injected hippocampi (bar=500 μm) and (b) Aβ immunostained area (total area) and number of Aβ-immunopositive deposits (n=8 biologically independent samples (APP/PS1 mice Con- (Con.sol.), ASC speck-injected (ASC specks)), n=4 biologically independent samples (non-injected (non-inj.) APP/PS1 mice), mean±SEM, one-way ANOVA, Tukey test, total area ASC speck vs con.sol ***p<0.0001. ASC specks vs non.-inj. ***p=0.0006, number of Aβ deposits ASC speck vs con. sol ***p=0.0003, ASC specks vs non.-inj. ***p<0.0001). (c) Immunoblots for APP, α and β c-terminal fragments (α-CTF, β-CTF) and Aβ from injected hemispheres. Brain lysates from non-injected (non-inj.) 6-month old APP/PS1 animals or wt mice as controls. (d) Quantification of the Aβ monomers (n=5 biologically independent samples, mean±SEM, one-way ANOVA, Tukey test, ASC speck vs con.sol ***p<0.0001, ASC specks vs non.-inj. ***p=0.0005). Determination of the rostrocaudal ASC speck-induced spreading of Aβ pathology. (e-h) Number of Aβ positive (+) deposits displayed for each section (e) Exp-I (c,f) Exp-II (g) Exp-III and (h) Exp-IV (EXP-1: n=7 biologically independent samples, EXP-II n=3 biologically independent samples, EXP-III: n=3 biologically independent samples, EXP-IV: n=5 biologically independent samples, mean±SEM, one-tailed Student's t-test, (levels from −4 to +4) EXP-I: −2 **p=0.0028, 1 *p=0.0194, 2 **p=0.061, 4 ***p=0.0007, EXP-II: −3 *p=0.0175, −2 *p=0.0216, 1 **p=0.0090, 2 *p=0.0312, EXP-III: −4 *p=0.0181, −2 *p=0.0194, 2 *p=0.0195, 3 **p=0.0072, EXP-IV: −4 ***p=0.0008, −3 *p=0.0037, −2 *p=0.0414, −1 *p=0.0144, 1 ***p<0.0001, 2 **p=0.0088, 3 **p=0.0012). (i) Representative scatter plots of animals analyzed for microglial amyloid content after intraperitoneal (i.p) injection of methoxy-XO4 (MxO4) and isolation of microglia at one month after injection. Wild-type mice (wt) isolation of microglial cell population without immunostaining for Cd11b/CD45 (upper panel) and after i.p. administration of methoxy-XO4 (lower panel). APP/PS1 mice receiving intrahippocampal injections with control solvent (upper panel) and ASC specks (lower panel) immunostained for CD11b/CD45/methoxy-XO4. Quantification of phagocytosis revealing no differences between groups (n=3 biologically independent animals, mean±SEM, two-tailed student's t-test). Experiments shown in a have been independently replicated twice, experiments shown in c five times. Experiments shown in i have been performed once.

FIG. 17 Lack of IDE and phagocytosis modulation in vivo seeding experiments. Representative scatter plots of animals analyzed for microglial amyloid content after intraperitoneal (i.p) injection of methoxy-XO4 (MxO4) and isolation of microglia one month after injection. (a) Analysis of microglial cell population (upper panel) from wild-type mice (wt) before and after i.p. administration of MxO4 (lower panel). APP/PS1 or APP/PS1/ASC^{-/-} mice (host animals:red) injected with either APP/PS1 or WT mouse brain homogenate (injection material: green). (b) Quantification of amyloid content revealed no differences between groups (n=3 biologically independent samples, mean±SEM, one-way ANOVA, Tukey test). (c) Enzymatic IDE activity was analyzed from mouse brain homogenates derived from EXP-IV using the FRET substrate (5-FAM/QXL520) and given as relative fluorescence units (RFU) per mg brain tissue. (EXP-I: n=7 biologically independent samples (APP/PS1 mice Con- (Con.sol.), ASC speck-injected (ASC specks)), n=4 biologically independent samples (non-injected (non-inj.) APP/PS1 mice), EXP-II n=4 biologically independent samples, EXP-III: 5=3 biologically independent samples, EXP-IV: n=6 biologically independent samples, mean±SEM, one-way ANOVA, Tukey test). Experiments shown in a were performed once.

FIG. 18 Cell viability study upon administration of increasing volumina of ASC specks. FIG. 18A depicts the experimental results (control versus increasing volumina of ASC specks). Resulting from the viability analysis (FIG. 18B), it may be seen that there was a concentration-dependent decrease of surviving neurons. It is thus concluded that primary neurons experience cell demise upon exposure to ASC specks. By using an anti ASC-speck antibody therapeutic approach, the neuro-protective effect is thus enhanced, as the amount of ASC specks being capable of aggregating Aβ is reduced. Loss of cell viability is thus reduced by anti-ASC speck antibodies.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Material and Methods

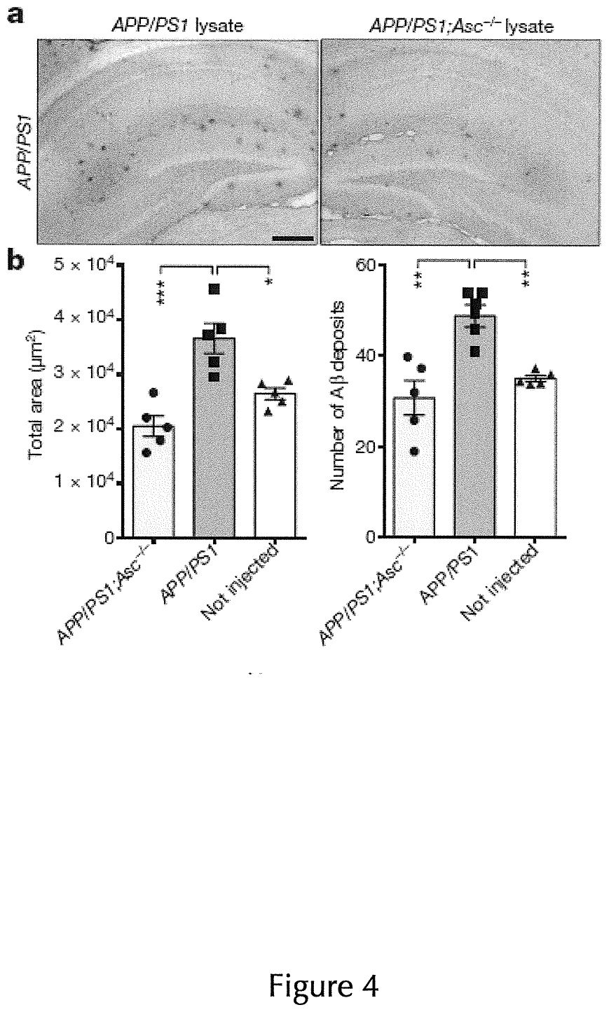

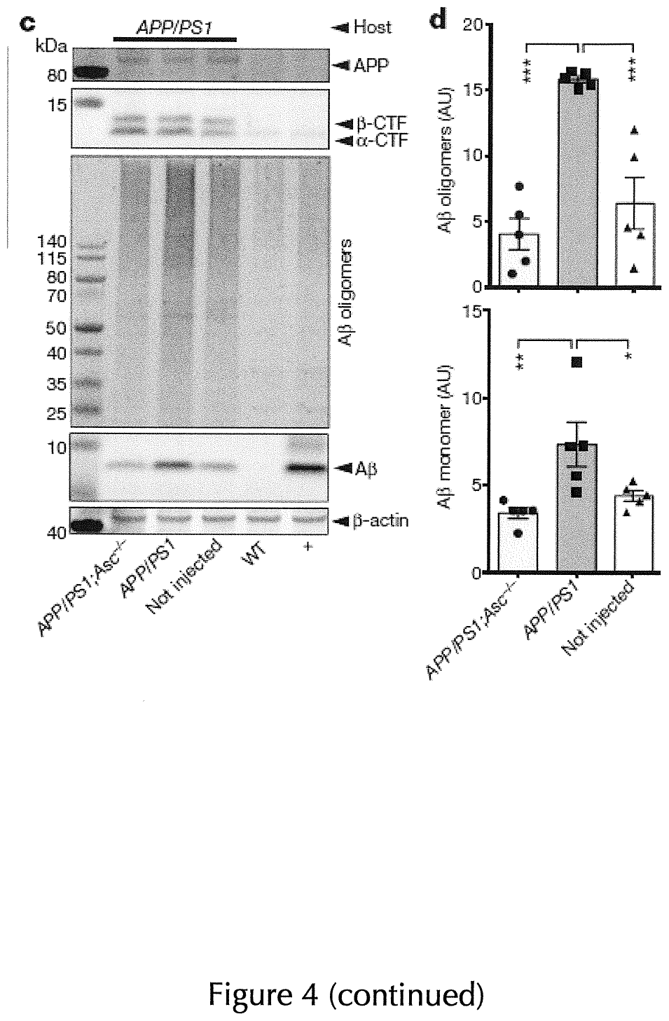

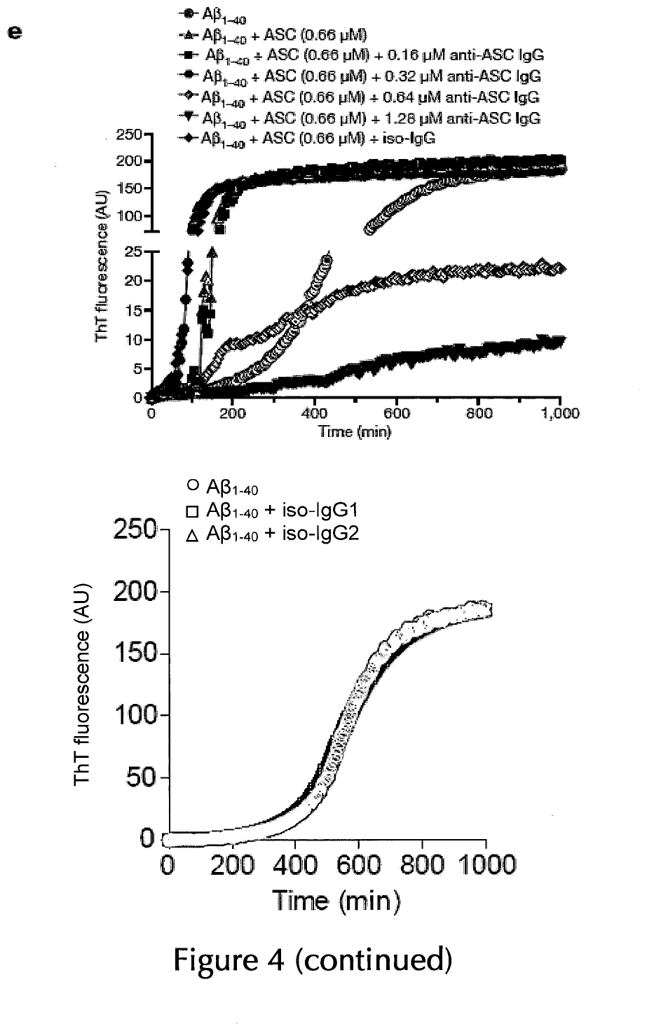

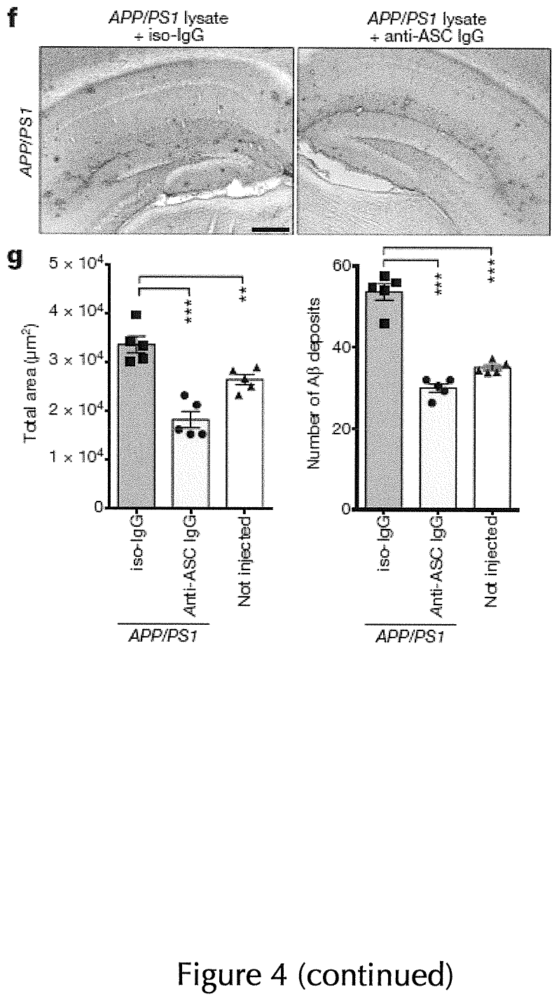

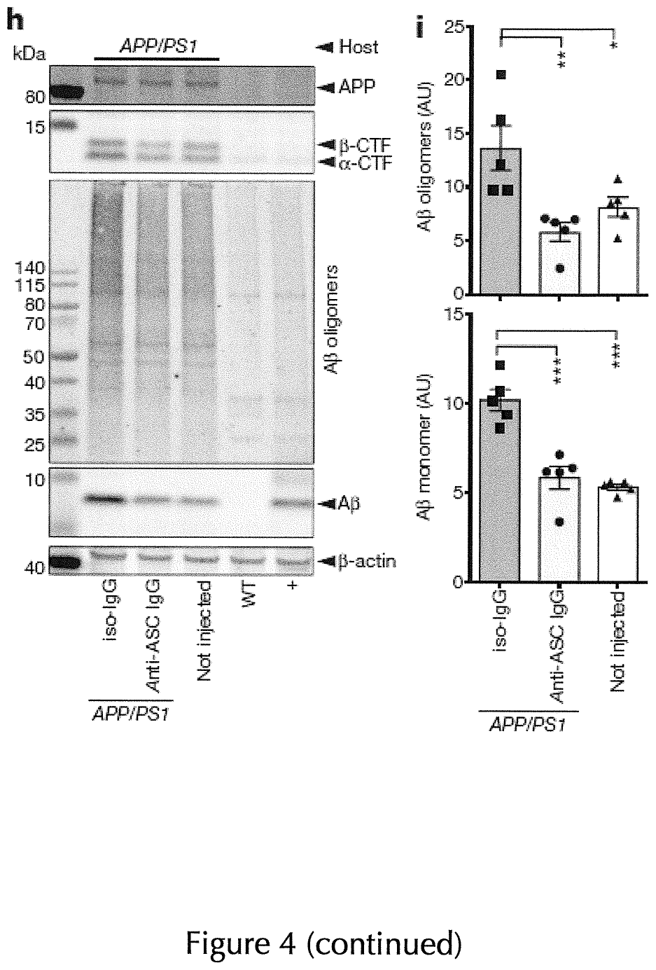

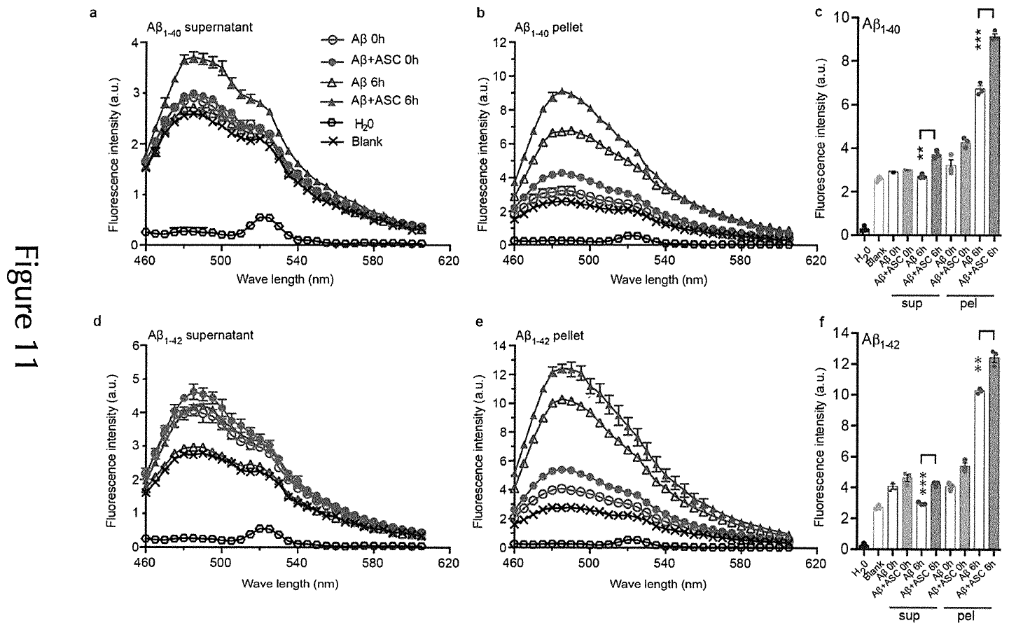

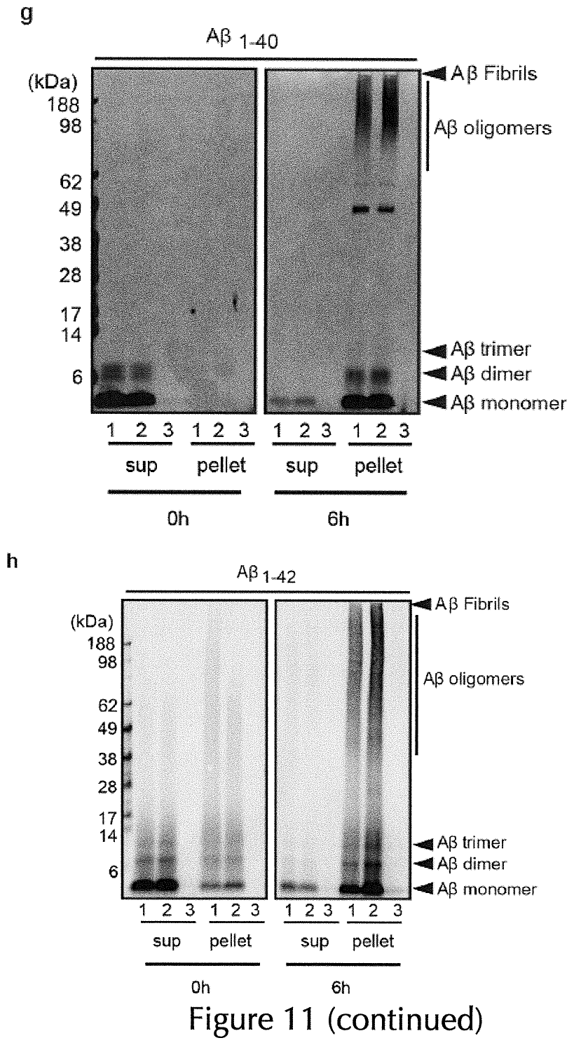

Reagents: Ultrapure LPS (E. coli 0111:B4) was from Invivogen (San Diego, Calif., U.S.A.); nigericin was from Invitrogen (Carlsbad, Calif., U.S.A.) and ATP was from Sigma-Aldrich (Munich, Germany). Antibodies to ASC were from BioLegend (San Diego, Calif., U.S.A., mAb, 653902, clone TMS-1, 1:500) and AdipoGen (ASC, AL177, AG-25B-0006-C100, Liestal, Switzerland). Purified mouse IgG1 (Invitrogen, 02-6100) and normal rabbit IgG (Santa Cruz Biotechnology, sc-2027, Heidelberg, Germany) were used as isotype control antibodies for the BioLegend ASC antibody and the AdipoGen ASC antibody, respectively.
Animals: APP/PS1 transgenic animals (The Jackson Laboratory, Bar Harbor, Me., U.S.A., strain #005864), and ASC^{-/-} animals (Millennium Pharmaceuticals, Cambridge, Mass., U.S.A.) were both on the C57B/6 genetic background. Mice were housed under standard conditions at 22° C. and a 12 h light-dark cycle with free access to food and water. Animal care and handling was performed according to the Declaration of Helsinki and approved by the local ethical committees (LANUV NRW #84-02.04.2017.A226). Only female animals were included in this analysis. Tissues of the following animal groups were analyzed: WT, ASC^{-/-}, APP/PS1, APP/PS1/ASC¹. Tissue from 8 m old APP/PS1 and APP/PS1/ASC^{-/-} mice served as non-injected controls for EXP-II, III and IV (Extended Data FIG. 11b). All animal experiments were performed by researchers blinded for the genotype of the animals. Power analysis were used to predetermine the sample size in case of in vivo studies. In the latter, animals were randomly assigned to the experimental conduct.
Human tissue samples: Post mortem brain material from histologically confirmed AD, vascular dementia (VD), frontotemporal dementia (FTD) and Corticobasal degeneration (CBD) cases as well as age-matched controls that had died from non-neurological disease, were derived from the Neurological Tissue Bank of the Biobank of the Hospital Clinic-IDIBAPS. All patients had signed an informed consent and agreed to the use of their brain material for medical research. Ages as well as post-mortem times were similar between controls and AD cases. Postmortem times varied from 3.5-5 hrs. After explantation, brain specimens were immediately snap frozen and stored at −80° C. until further use. Patients and controls were 75*6 yrs old.
Immunohistochemistry (ASC/CD11b/A3) in mice and men: Free-floating 40-μm thick serial sections were cut on a vibratome (Leica, Wetzlar, Germany). Sections obtained were stored in 0.1% NaN₃, PBS. For immunohistochemistry, sections were treated with 50% methanol for 15 min then washed 3 times for 5 min in PBS and blocked in 3% BSA, 0.1% Triton X-100, PBS (blocking buffer) for 30 min followed by overnight incubation with the primary antibody in blocking buffer. Sections were washed 3 times in 0.1% Triton X-100, PBS and incubated with Alexa 488 or Alexa 594 antibody conjugates (1:500, Invitrogen, Eugene, Oreg., USA) for 90 min, washed 3 times with 0.1% Triton X-100, PBS for 5 min. Sections were mounted using Immu-Mount (9990402, Thermo Scientific, Cheshire, UK). The following primary antibodies were used with respective concentrations: rat anti-mouse CD11b (1:200, MCA711, Serotec, Oxford, UK), rabbit anti-mouse ASC (1:200, AL177, AG-25B-0006-C100, AdipoGen, Liestal, Switzerland) and Aβ anti-human (1:400, 6E10, SIG-39320, Covance, Munster, Germany).
Quantification of intra- and extracellular ASC specks: For human subjects, 10 controls and 10 AD cases were analyzed. From each patient, 6 hippocampal brain sections with a defined distance to each other were evaluated. Intra- and extracellular ASC specks were counted in 10 randomly chosen fields per section at a 40× magnification. Similarly, hippocampal sections of WT and APP/PS1 mice were analyzed at 2, 4 and 8 months of age. The proportion of intra- or extracellular ASC specks was given as intracellular or extracellular ASC speck per microglia or percentage of all ASC specks detected.
Cell culture: Primary microglial cell cultures were prepared as previously described in detail²⁶. Briefly, mixed glial cultures were prepared from newborn wt mice and cultured in DMEM (31966, ThermoFisher, Darmstadt, Germany) supplemented with 10% FCS (10270, ThermoFisher, Darmstadt, Germany) and 100 U/ml penicillin/streptomycin (15070, ThermoFisher, Darmstadt, Germany). Microglial cells were used after 14 days of primary cultivation. They were harvested by shake off, re-plated and allowed to attach to the substrate for 30-60 min. To assess the release of ASC specks, unstimulated, or LPS-primed microglia were left untreated, or activated with nigericin (10 μM) or ATP (5 mM). Cells were fixed, washed and stained with anti-ASC (1:100 BioLegend, clone HASC-71), or purified IgG1 (02-6100, ThermoFisher, Darmstadt, Germany), followed by staining with goat anti-Mouse-Alexa Fluor 488 (A-11017, ThermoFisher, Darmstadt, Germany). The monocytic cell line THP-1 stably transduced with constructs for the expression of mCerulean-ASC has been described¹⁶. Cells were cultured in RPMI 1640 supplemented with 10% FBS and penicillin/streptomycin. For stimulation assays, cells were treated with 100 nM of phorbol 12-myristate 13-acetate (PMA, Sigma-Aldrich, Munich, Germany) overnight, primed with 1 μg/ml of LPS for 3 h and further activated with 10 μM of nigericin for 90 min. Mycoplasma contamination has been excluded by regular testing.
FACS analysis of ASC speck release: The quantification of ASC specks in cell-free supernatants of microglia was carried out on a MACSQuant analyzer (Miltenyi Biotec, Bergisch Gladbach, Germany), after gating on debris-sized events using micro sized beads of 0.7-0.9 μm (Spherotech, Lake Forest, Ill., U.S.A.) or 6.0 μm (BD Biosciences, Heidelberg, Germany) as reference for their distribution on a FSC vs. SSC scatter. Cell-free supernatants were stained with anti-ASC (clone TMS-1, 1:500, BioLegend, San Diego, Calif., U.S.A.), or an equivalent amount of purified IgG1 isotype (02-6100, ThermoFisher, Darmstadt, Germany) directly conjugated to Alexa Fluor 488 dye. Debri-sized A488⁺ events were counted as ASC specks. Data were analyzed with FlowJo X 10.0.7 (Ashland, Oreg., U.S.A.).
Confocal laser scanning microscopy: Microglia or THP-1 cells were imaged in a Leica TCS SP5 SMD confocal system (Leica Microsystems, Wetzlar, Germany). Images were acquired using a 63× objective, with a numerical aperture of 1.2, and analyzed using the Volocity 6.01 software (PerkinElmer, Waltham, Mass., USA).
Association of ASC specks with Aβ: To image the association of ASC specks with A01-42 in vitro, PMA treated (100 nM), LPS-primed (1 μg/mL) ASC-mCerulean expressing THP-1 were activated with nigericin (10 μM) for 90 min in the presence of soluble TAMRA-Aβ (PSL, Heidelberg, Germany). Cells were imaged at 37° C. with 5% CO₂ using an environmental control chamber (Life Imaging Services and Solent Scientific). Images were acquired using a 63× objective, with a numerical aperture of 1.2, and analyzed using the LAS AF version 2.2.1 (Leica Microsystems) or Volocity 6.01 software.
Generation and isolation of ASC specks. Generation and isolation of ASC specks were performed essentially as described previously^{16,27,28}. Inflammasome reporter macrophages were cultured in 15 cm dishes until they reached 80% confluence. Cells were harvested with a cell scraper in 5 ml PBS and pelleted by centrifuging (400×g/5 min). To remove residual medium, they were resuspended in 1 ml PBS and transferred to 1.5 ml Eppendorf tubes and centrifuged again at 1500 rpm/5 min at 4° C. Supernatants were removed and the pellets put to −80° C. for at least 15 min to destabilize the cytoplasmic membranes. Afterwards cells were resuspended in 2× volume of CHAPS buffer and lysed using a 2 ml syringe with a 20 G needle. To remove cellular debris the samples were centrifuged (14,000 rpm/8 min/4° C.) and supernatants transferred to sterile 1 ml polycarbonate ultracentrifuge tubes (Beckmann) and spun down at 100,000 g in a Beckman Optima TLX benchtop ultracentrifuge for 30 min to obtain S100 supernatants. These supernatants were transferred to 0.5 ml PVDF 0.22 μm filter tubes and filtered by centrifugation (14000 rpm/5 min/4° C.). The flow through was incubated at 37° C. for 60-90 min to induce the assembly of ASC specks. ASC specks were treated with TEV for 1 h at 4° C., and washed twice in PBS before used in experiments (Extended data FIG. 4).
Preparation of recombinant ASC from E. coli The cDNA encoding full-length human ASC followed by a TEV protease cleavage site and mCherry was cloned into the pET-23a expression vector providing a C-terminal hexa-histidine tag (pET23a-ASC-Tev-mCherry-His). The plasmid was transformed into Escherichia coli BL21 (DE3) cells. Transformed E. coli cells were grown at 37° C. and expression was induced at an OD₆₀₀ of 0.8 by 1 mM isopropyl R-D-1-thiogalactopyranoside for 4 h. The cells were harvested by centrifugation and sonicated in a buffer containing 20 mM Tris (pH 8.0), 500 mM NaCl, 5 mM imidazole (buffer A). The cell lysate was centrifuged for 30 min at 20,000 rpm at 4° C. The cell pellet was resuspended in buffer A supplemented with 2 M guanidine-HCl and centrifuged and the supernatant was dialysed (visking dialysis tubing, cellulose, type 36132, MWCO 14,000 Daltons; Carl Roth, Karlsruhe, Germany) against buffer A at 4° C. The sample was again centrifuged and the supernatant was administered onto a pre-equilibrated HisTrap column using an Akta Prime FPLC system (GE Healthcare). The column was washed with 10 column volumes of 20 mM Tris (pH 8.0), 500 mM NaCl, 20 mM imidazole, and the protein was eluted in the same buffer containing 200 mM imidazole. The purified protein was dialysed against a buffer containing 20 mM Tris (pH 8.0), 300 mM NaCl. To induce fibrillation of the ASC-mCherry chimeric protein, the solution was centrifuged at 100,000 g for 1 h at 4° C. and subsequently incubated for 1 h at 37° C. Samples were kept on ice and immediately subjected to further analyses avoiding freeze/thaw cycles. Besides the wild-type ASC protein, five mutants were generated. These mutants were designed to break the homomeric oligomerization interface in either the PYD or the CARD only, or in both domains. Mutant sites were identified based on structural analyses of domain fibrillation^{1,2}. K to E mutations of the PYD-PYD assembly interface (K21E, K22E, K26E), K to A of the same interface (K21A, K22A, K26A), D to R and Y to E of the putative CARD assembly interface (D134R, Y187E), and the two combinations K to E/D to RN to E (K21E, K22E, K26E, D134R, Y187E) and K to A/D to RN to E (K21E, K22E, K26E, D134R, Y187E). All protein expression constructs were confirmed by sequence analysis. Protein expression, purification, and preparation and the protocol applied for fibrillation was the same as for the wild-type protein.
FACS analysis of Aβ and ASC specks from supernatants of immunostimulated murine microglia and macrophages. Primary murine microglia and immortalized ASC-mCerulean and macrophages with and without genetic deficiency for ASC were primed with 200 ng/ml of LPS for 3 h in 100 pI complete media. Subsequently, the NLRP3 inflammasome was activated by adding 5 mM of ATP for 60 minutes. The supernatants were removed and incubated with TAMRA-labeled Aβ for 6 h at 37° C. and subsequently stained with Alexa Fluor 647 anti-ASC (ThermoFisher, Darmstadt, Germany) overnight at 4° C. Thereafter, FACS analysis was performed with a MACSQuant (Miltenyi Biotec).
Immunoblot of Aβ oligomer formation: Synthetic Aβ₁₋₄₂ was procured from Peptide Specialty Laboratories (PSL, Heidelberg, Germany). Lyophilized peptide was solubilized in 10 mM NaOH to a final concentration of 1 mg/ml (221 μM), sonicated for 5 min in a water bath and stored at −80° C. Aβ₁₋₄₂ was diluted to 100 μM in 50 mM Sorenson's phosphate buffer, pH 7.0. Aβ₁₋₄₂ was incubated with and without ASC specks (0.53 μM) at 37° C. for 24 h. Samples were collected at 0, 1, 2, 4, 6 and 24 h. Samples were separated on a 4-12% NuPAGE by electrophoresis and transferred onto nitrocellulose membrane. The membrane was blocked with 5% milk in PBS, 0.05% Tween 20 (blocking solution) and incubated overnight at 4° C. with 6E10 antibody (SIG-39320, Covance, Munster, Germany) in blocking solution. The membrane was incubated with the antibody conjugates and the immunoreactivity was detected using the Odyssey Clx imaging system (Li-COR, Bad Homburg, Germany).
Immunoblotting of murine brain lysates. Samples were separated by 4-12% NuPAGE (Invitrogen, Karlsruhe, Germany) using MES or MOPS buffer and transferred to nitrocellulose membranes. For caspase-1 blots, positive controls were generated by precipitating supernatants from wild-type immortalized murine macrophages, which were treated with 200 ng/ml LPS for 3 h, followed by 10 μM nigericin for 1 h. APP and Aβ were detected using antibody 6E10 (Covance, Munster, Germany) and the c-terminal APP antibody 140²⁹ (CT15). IDE was blotted using antibody PC730 (Calbiochem, Darmstadt, Germany), caspase-1 using antibodies casp-1 clone 4B4.2.1 (gift from Genentech, San Francisco, Calif.) and a caspase-1 antibody raised in rabbit (gift from Gabriel Nuhez), neprilysin using antibody 56C6 (Santa Cruz, Heidelberg, Germany), and β-actin using A2228 (Sigma, Munich, Germany) and 926-42212 (LI-COR Biosciences, Bad Homburg, Germany). Immunoreactivity was detected by enhanced chemiluminescence reaction (Millipore, Darmstadt, Germany) or near-infrared detection (Odyssey, LI-COR). Chemiluminescence intensities were analyzed using Chemidoc XRS documentation system (Biorad, Munich, Germany). Positive controls for NEP (recombinant Mouse NEP protein; 1126-ZN) and IDE (recombinant IDE protein; 2496-ZN) were from R&D systems (R&D System, Inc. Minneapolis, Minn., USA).

Thioflavin T fluorescence assay: Synthetic Aβ₁₋₄₀ and Aβ₁₋₄₂ peptides were procured from Peptide Specialty Laboratories (PSL, Heidelberg, Germany). Lyophilized peptides were solubilized in 10 mM NaOH to a final concentration of 1 mg/ml, sonicated for 5 min in a water bath (Brandelin Sonopuls, Berlin, Germany) and stored at −80° C. until further use. For monitoring Aβ-fibrillization, Thioflavin T (ThT) binding assay was performed as described previously³⁰. Briefly, Aβ stock solution was diluted to final Aβ concentration of 50 μM in ThT fluorescence assay buffer (50 mM sodium phosphate buffer (pH 7.4), 50 mM NaCl, 20 μM ThT, and 0.01% sodium azide). Real time ThT fluorescence measurements were carried out using a Varian Cary Eclipse fluorescence spectrophotometer (Agilent, Waldbronn, Germany). Samples were incubated at 37° C. with stirring. The ThT fluorescence was measured every 5 min for 25 hours at excitation and emission wavelengths of 446 nm and 482 nm, respectively, with a slit width of 5 nm. To assess cross-seeding of Aβ fibrillization, freshly diluted Aβ₁₋₄₀ and Aβ₁₋₄₂ (50 μM) were incubated with ASC specks purified from ASC expressing cells (0.22 and 1.75 μM) at 37° C. with stirring. Real time ThT fluorescence measurements were carried out as described above. The cross-seeding effect of ASC specks was also assessed on TAMRA-labeled Aβ₁₋₄₂ and Aβ₄₂₋₁ peptides.

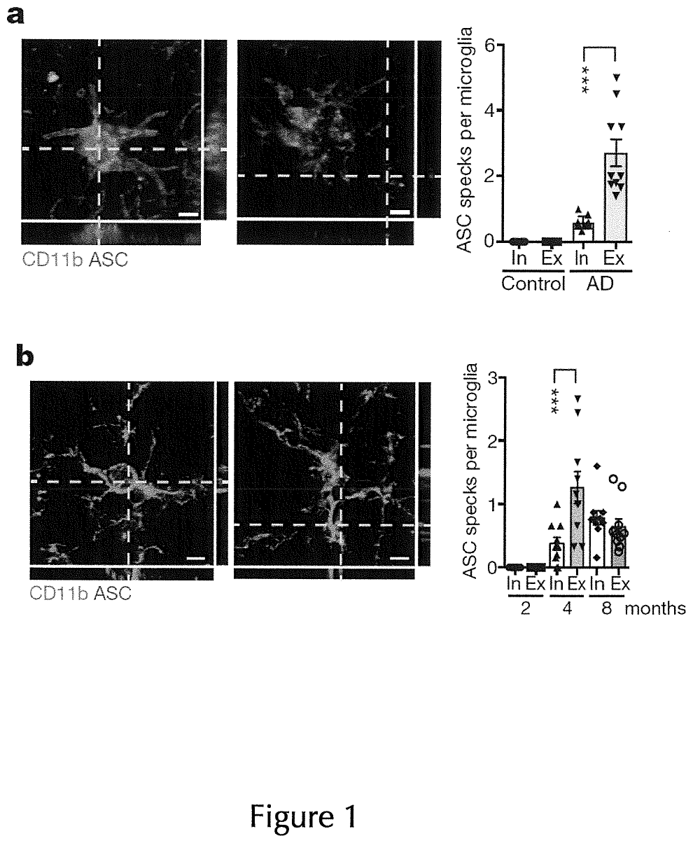

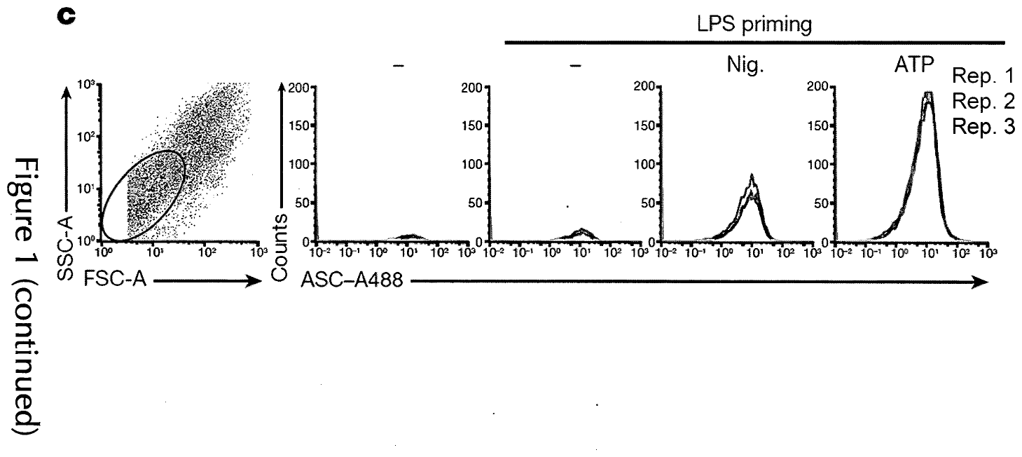

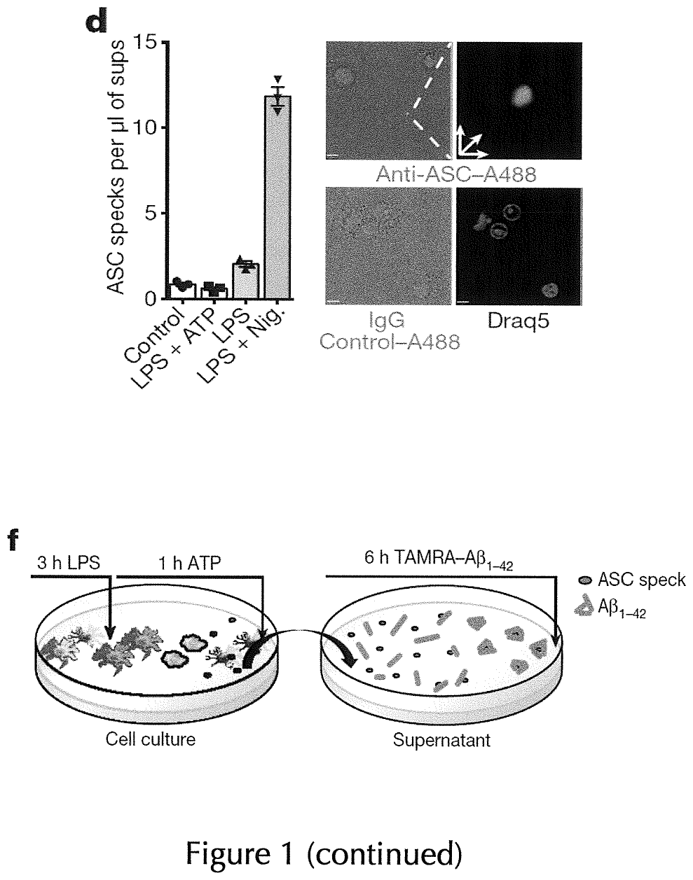

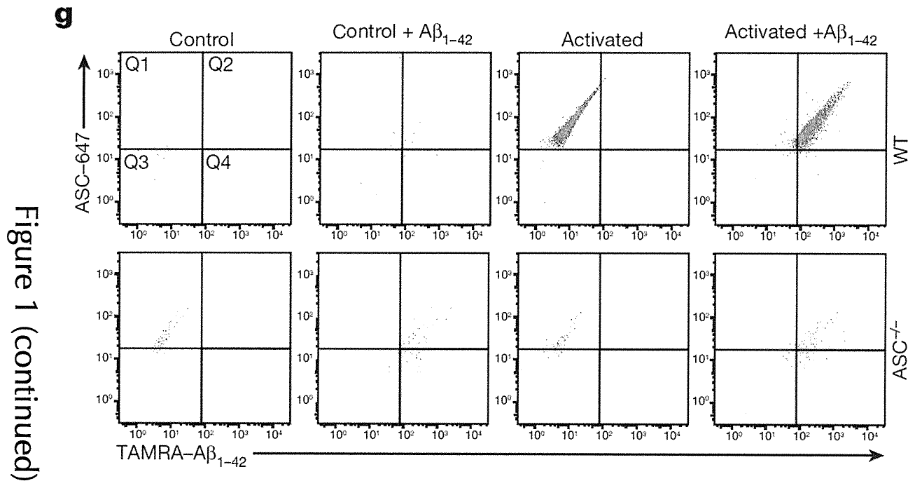

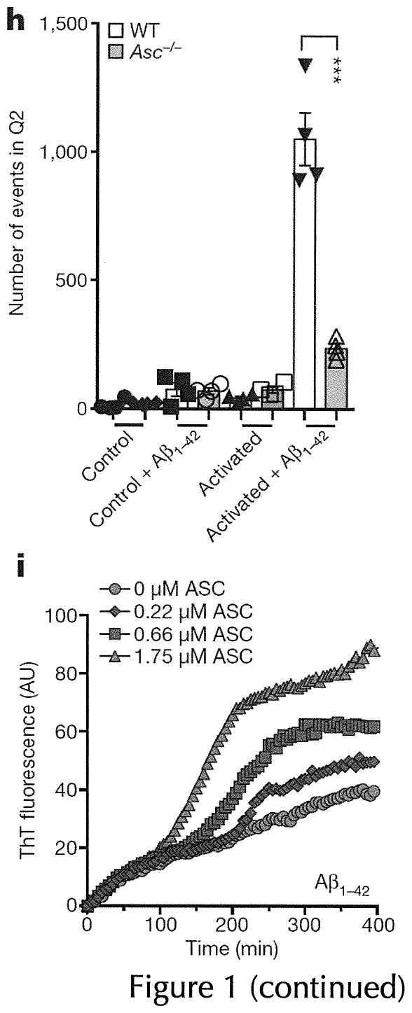

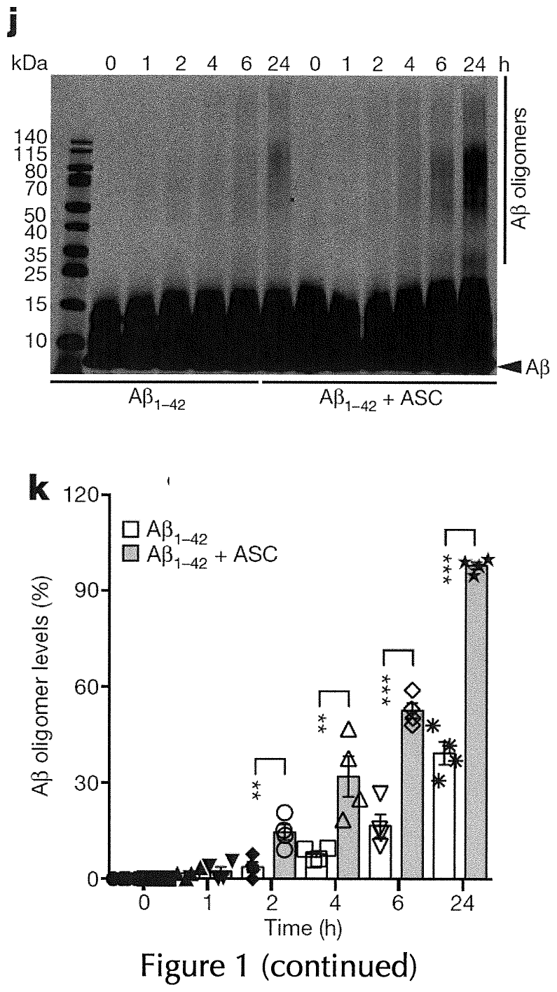

Turbidity assay: For turbidity measurements, sample aliquots collected at the end of the aggregation assays were used. Absorbance was measured using an Agilent 8453 UV spectrophotometer set at a wavelength of 403 nm.
Interaction of A with recombinant ASC protein: Recombinant ASC protein alone (without Aβ) and monomeric Aβ₁₋₄₀ and Aβ₁₋₄₂ solutions (50 μM) supplemented with or without recombinant ASC protein (2 μM) were incubated at 37° C. with shaking up to 96 hrs. Sample aliquots collected at various time intervals (0, 12, 24, 48, 72 and 96 h) were subjected to electron microscopy and SDS-PAGE electrophoresis. After SDS-PAGE, Western blot analysis was performed using anti-ASC speck and anti-Aβ antibodies employing the Odyssey Clx imaging system (Li-COR, Bad Homburg, Germany) Quantification was performed using Li-COR Image Studio Software (Li-COR, Bad Homburg, Germany).
Transmission electron microscopy: 1 mg of lyophilized amyloid- (1-42) peptide (PSL, Germany) was dissolved in 250 μl NaOH and 750 μl Tris/HCl (pH 7.6) buffer to a final concentration of 1 mg/ml. The sample was incubated for 2 h at 37° C. and afterwards centrifuged at 20,000 g for 5 min. A was then mixed with ASC protein and incubated in time course experiments up to 72 h. Samples of either ASC-mCherry, A, or ASC-mCherry together with A were bound to carbon-coated grids and stained with 1% uranyl acetate. Pictures were taken at 72,000× magnification at a CM120 microscope with a 4096×4096 pixel TemCam (Tietz, Gauting, Germany) in spotscan mode.
Immunoprecipitation experiments: Human or mouse brain samples were homogenized in NP-40 buffer with inhibitors (AEBSF, protease inhibitor cocktail (Sigma-Aldrich, Munich, Germany), NaF and NaVO₃). 60 μl of protein G magnetic beads were washed 3 times in 1 ml PBS, 0.1% Tween 20 and incubated with anti-ASC or 6E10 antibodies for 10 min at room temperature while rotating. Beads were washed 3 times in 1 ml 0.1% PBS-T. Samples were added and incubated for 1 h at room temperature while rotating. Samples were washed 3 times in PBS, 0.1% Tween 20, resuspended in 4× NuPAGE sample buffer, heated for 10 min at 70° C. and centrifuged at 14000×g for 5 min The supernatants were separated by 4-12% NuPAGE and analysed by Western blot.
Aβ-ASC specks co-sedimentation analysis: Aβ-ASC specks co-sedimentation analysis was performed employing purified ASC specks and synthetic Aβ peptide. Monomeric Aβ₁₋₄₀ and Aβ₁₋₄₂ solutions (50 μM) were incubated with or without ASC specks (1.75 μM) at 37° C. with shaking. ASC specks without Aβ in the respective buffers were used as controls. For quantitative sedimentation analysis, sample aliquots collected at different time intervals (0.25 h and 6 h) were fractionated into supernatants and pellets were subjected to ultracentrifugation (100,000×g, 1 h, 4° C.). The resulting pellets were resuspended in a volume of buffer corresponding to the volume of supernatant. The supernatant and pellet fractions were electrophoresed on 4-12% NuPAGE (Invitrogen, Karlsruhe, Germany) gradient gels under denaturing and reducing conditions. Western blot analysis was performed using anti-ASC speck and anti-Aβ antibodies employing Odyssey Clx imaging system (Li-COR, Bad Homburg, Germany) Quantification was performed using Li-COR Image Studio Software (Li-COR, Bad Homburg, Germany). The formation of -sheet rich oligomers/fibrils were quantified by ThT fluorescence assay. Fluorescence spectra of the Aβ₁₋₄₀ and Aβ₁₋₄₂ supernatants and pellet fractions with and without ASC specks were monitored at λemission between 460 and 605 nm with excitation at 446 nm. Excitation and Emission slit set at 10 nm. The λmax emission values (485 nm) of supernatants and pellet fractions at 0.25 h and 6 h intervals were used for the statistical analysis.
Behavioural phenotyping: Morris WaterMaze test. Spatial memory testing was conducted in a pool consisting of a circular tank (Ø1 m) filled with opacified water at 24° C. The water basin was dimly lit (20-30 lux) and surrounded by a white curtain. The maze was virtually divided into four quadrants, with one containing a hidden platform (15×15 cm), present 1.5 cm below the water surface. Mice were trained to find the platform, orientating by means of three extra maze cues placed asymmetrically as spatial references. They were placed into the water in a quasi-random fashion to prevent strategy learning. Mice were allowed to search for the platform for 40 s; if the mice did not reach the platform in the allotted time, they were placed onto it manually. Mice were allowed to stay on the platform for 15 s before the initiation of the next trial. After completion of four trials, mice were dried and placed back into their home cages. Mice trained 4 trials per day for 8 consecutive days. The integrated time or distance travelled was analyzed per animal with baseline levels set for area under the curve calculations (AUC, latency 10 s, distance 100 cm). For spatialprobe trials, which were conducted 24 h after the last training session (day 9), the platform was removed and mice were allowed to swim for 30 s. The drop position was at the border between the 3^{rd} and 4^{th} quadrant, with the mouse facing the wall at start. Data are given as percent of time spent in quadrant Q1, representing the quadrant where the platform had been located, and compared to the averaged time the animals spent in the remaining quadrants. In the afternoon of the same day, a visual cued testing was performed with the platform being flagged and new positions for the start and goal during each trial. All mouse movements were recorded by a computerized tracking system that calculated distances moved and latencies required for reaching the platform (Noldus, Ethovision 3.1).
Murine and human Aβ plaque analysis: Amyloid plaque cores were isolated according to a previously published method^{32XX31}. Briefly, mouse brain hemispheres or human brain samples were homogenized, boiled in 2% SDS, 50 mM Tris-HCl pH 7.5, 50 mM DTT, and centrifuged at 100,000×g for 1 h at 10° C. The pellet was solubilized in 1% SDS, 50 mM Tris-HCl pH 7.5, 50 mM DTT and centrifuged at 100,000×g for 1 h at 10° C. The pellet was suspended in 1% SDS, 50 mM Tris-HCl pH 7.5, 50 mM DTT and loaded on top of a discontinuous sucrose gradient (1.0, 1.2, 1.4 and 2.0 M sucrose in 50 mM Tris pH 7.5 containing 1% SDS), centrifuged at 220,000×g for 20 h at 10° C. and fractionated into 6 fractions. Amyloid plaque cores were found to be enriched at the 1.4/2 M interface. Samples were analyzed by immuno dot blot using antibodies 6E10 or Alz-177 (Invivogen, San Diego, Calif.) against ASC.
ELISA quantification of cerebral Aβ concentrations. Quantitative determination of Aβ was performed using an electrochemiluminescence ELISA for Aβ₁₋₃₈, Aβ₁₋₄₀ and Aβ₁₋₄₂ (Meso Scale Discovery, Gaithersburg, Md., USA). Signals were measured on a SECTOR Imager 2400 reader (Meso Scale Discovery, Gaithersburg, Md., USA). Plates were blocked with 5% blocker A (Meso Scale, Gaithersburg, Mass.), 0.1% mouse gamma globulin (Rockland, Gilbertsville, Pa.). SDS and FA fractions from mouse brain were diluted in 1% blocker A, 0.1% mouse gamma globulin 1:25 and 1:100, respectively. 30 μl samples were incubated for 4 h at RT, washed with Tris wash buffer (Meso Scale, Gaithersburg, Mass.) and incubated with 0.25 μg/ml MSD-tagged antibody 4G8 (Meso Scale, Gaithersburg, Mass.) diluted in 1% blocker A, 0.1% mouse gamma globulin for 1 h at RT. Wells were washed with Tris wash buffer. Detection wash conducted in 150 μl of 2× read buffer (Meso Scale, Gaithersburg, Mass.) was added.
Stereotaxic surgery Three-month old host mice were anesthetized with an intraperitoneal injection of ketamine (0.10 mg/g body weight) and xylazine (0.01 mg/g body weight). Animals were placed into a stereotactic mouse frame (Stoelting, Wood Dale, Ill., U.S.A.) equipped with a heating blanket to maintain body temperature at 37° C. throughout the procedure. Two small holes were drilled into the skull using a Dremel device adapted to the stereotactic frame. Thereafter host animals received a bilateral stereotaxic injection of either 2 μl ASC specks or control (Extended Data FIG. 11b,f). Exp I, host: APP/PS1 mice), brain extract prepared from APP/PS1 or WT mice (Extended Data FIG. 11b,f, Exp.II, host: ASC^{-/-}, APP/PS1, APP/PS1/ASC^{-/-}), brain extract prepared from APP/PS1 or APP/PS1/ASC^{-/-} mice (Extended Data FIG. 11b,f, Exp III, host: APP/PS1) or were injected with brain extract prepared from APP/PS1 mice containing either anti-ASC-IgG or isotype-IgG (Extended Data FIG. 11b,f, Exp IV, host: APP/PS1) using Hamilton syringes into the hippocampus at AP −2.5 mm, L +/−2 mm, DV −1.8 mm. Injection speed was pump controlled at 0.5 μl/min. The needle was kept in place for an additional 10 minutes before it was slowly withdrawn to avoid reflux up the needle tract. Skull holes were filled carefully with sterilized bone wax. Then, the operation field was again cleaned and the incision was sutured. All mice were monitored until complete recovery from anaesthesia. Subsequently, animals were housed under standard conditions until their sacrifice in IVC cages.
Animal perfusion: The animals were anaesthetized intraperitoneally with ketamine/xylazine (100 mg/kg and 10 mg/kg respectively) solution and then transcardially perfused with cold PBS (30 ml). The brains were removed from the animals and stored for 24 h in 4% paraformaldehyde (PFA) solution at 4° C. followed by washing 3 times with PBS and stored in PBS-NaN, until further use.
Tissue extracts: Mouse brain homogenates were prepared from APP/PS1, APP/PS1/ASC^{-/-} and WT forebrains (without cerebellum) of aged animals (16 months-old) following the method described by^{23,32} (see also Extended Data FIG. 11e). Brain tissue samples were snap-frozen in liquid nitrogen and stored at −80° C. until use. The tissue was homogenized (10% w/v) in sterile PBS. Aliquots of brain homogenates from APP/PS1 and APP/PS1/ASC^{-/-} mice were adjusted for equal amounts of Aβ by addition of wild-type mouse brain homogenate according to the results from ELISA measurements for Aβ₁₋₄₂. Aliquots were analyzed for Aβ content by immunoblot using antibody 82E1 and anti-actin antibody to normalize for protein loading. Homogenates were centrifuged at 3000 g for 5 min at 4° C., aliquoted and stored at −80° C. before use.
Analysis of Aβ plaque deposits: Free-floating 40-μm thick serial sections were cut on a vibratome (Leica, Wetzlar, Germany). Sections were stored in 0.1% NaN₃, PBS. For immunostaining, 8 sections per animal with defined distance to each other (Extended Data FIG. 11f) were fixed to slides and washed 3 times for 5 min in PBS, 10 min in PBS 0.1% Triton X-100, and 3% H₂O₂ in PBS for 15 min. They were washed for 5 min in PBS and blocked in 3% BSA, 0.1% Triton X-100, PBS (blocking buffer) for 1 h followed by overnight incubation with IC16 (1:400) antibody³³ in blocking buffer. Slides were washed 3 times in PBS and incubated with secondary antibody in blocking buffer for 2 h. Samples were washed 3 times for 5 min with PBS, and incubated with the A+B solution in PBS (1:50) (ABC Vectastain Elite Kit Lsg, Vector Laboratories, Burlingame, Calif.) for 30 min and washed 3 times for 5 min in PBS. Samples were incubated for 30 seconds in diaminobenzidine solution (0.17 mM diaminobenzidine, 0.01% H₂O₂ in PBS) and the reaction was stopped with water after 5 min. Sections were mounted using Immu-Mount (Thermo Scientific, Cheshire, UK). Bright field microscopy was conducted on an Olympus BX61 bright field microscope and images were processed with ImageJ.
Brain protein extraction: Snap-frozen brain hemispheres were extracted as previously described². Briefly, hemispheres were homogenized in PBS, 1 mM EDTA, 1 mM EGTA, 3 μl/ml protease inhibitor mix (Sigma, Munich, Germany). Homogenates were extracted in RIPA buffer (25 mM Tris-HCl, pH 7.5, 150 mM NaCl, 1% NP40, 0.5% NaDOC, 0.1% SDS), centrifuged at 100,000×g for 30 min and the pellet containing insoluble Aβ was solubilized in 2% SDS, 25 mM Tris-HCl, pH 7.5. In addition, the SDS-insoluble pellet was extracted with 70% formic acid in water. Formic acid was removed using a speed vac (Eppendorf, Hamburg, Germany) and the resulting pellet was solubilized in 200 mM Tris-HCl, pH 7.5.
IDE activity: IDE activity in mouse brain homogenates was measured using the SensoLyte® 520 IDE Activity Assay Kit (AnaSpec, Fremont, Calif.) according to the manufacturer's instructions, using the FRET (Fluorescence resonance energy transfer) substrate (5-FAM/QXL520). When active IDE cleaves the FRET substrate it results in an increase of 5-FAM (5-carboxyfluorescein) fluorescence, which was measured at an excitation wavelength of 490 nm and an emission wavelength of 520 nm, on an Infinite 200 PRO plate reader (Tecan, Männedorf, Switzerland). The total IDE activity was calculated using the equation,

$$IDE activity = \frac{A 1 - A 0}{C} \times D .$$

Where A1 is the concentration of 5-FAM at 30 min and A0 at 0 min; C is the total protein concentration and D is the dilution. The relative fluorescence units (RFU) of 5-FAM were normalized per mg of total protein that was determined using BCA reagent (Thermo Scientific, Rockford, USA).
Assessment of Aβ phagocytosis by FACS: To determine the phagocytic activity, 3 month-old APP/PS1 or APP/PS1/ASC^{-/-} were injected with APP/PS1 and WT lysate or ASC specks and control cell lysate. After 1 month, the animals were injected with 10 mg/kg Methoxy-XO4 (863918-78-9, TOCRIS bioscience, Bristol, UK) in 50% DMSO/50% NaCl (0.9%) pH=12 and 3 hours later they were analyzed as previously described¹². The microglia population was isolated from mice as previously described¹² and incubated with CD11b-APC (101212, BioLegend, Fell, Germany) and CD45-FITC (11-0451-82, eBioscience, Frankfurt, Germany) and Methoxy-XO4-positive, phagocytic microglia were determined by flow cytometry (FACS Canto II, BD Biosciences, Heidelberg Germany). Data were analyzed using FlowJo X 10.0.7 (FlowJo, Ashland, Oreg.).
Statistics: Data were analyzed either by one way ANOVA, followed by post hoc analysis where appropriate or by two-tailed, unpaired Student's t-test if not indicated otherwise, using Graph Pad Prism 6 for Mac OS or R. Statistical details are given in the respective figure legends.
Data availability: The datasets generated during and/or analysed during the current study have been made available as supplemental information (Supplemental FIG. 1-3) and xs.files. Further data are available on reasonable request to the corresponding author.

### Example 1: ASC Specks Enhance Aβ Aggregation

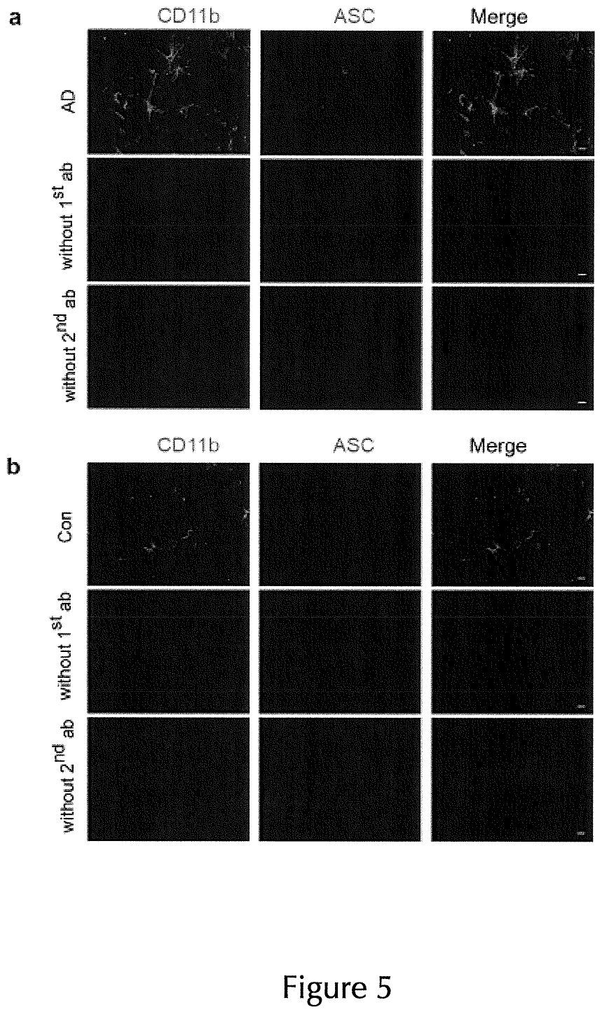

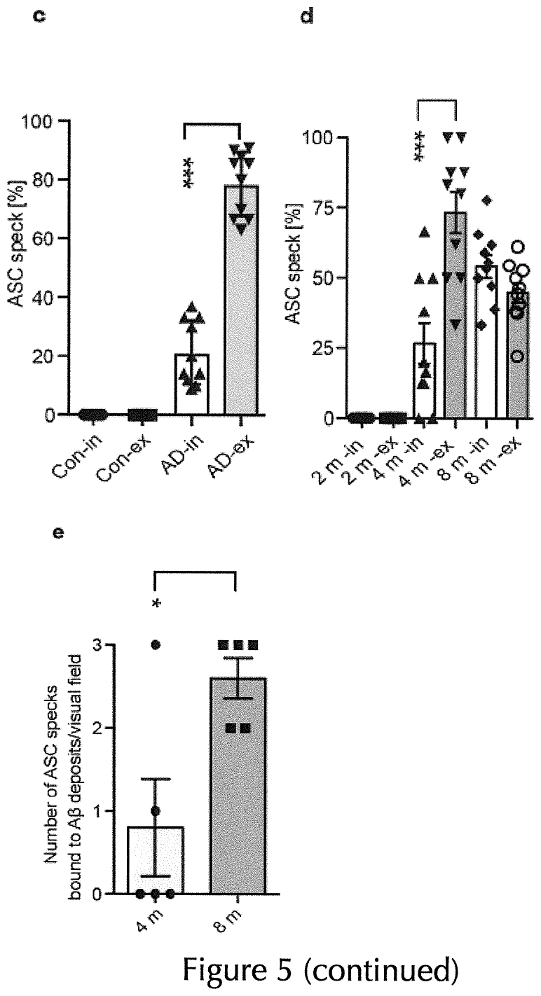

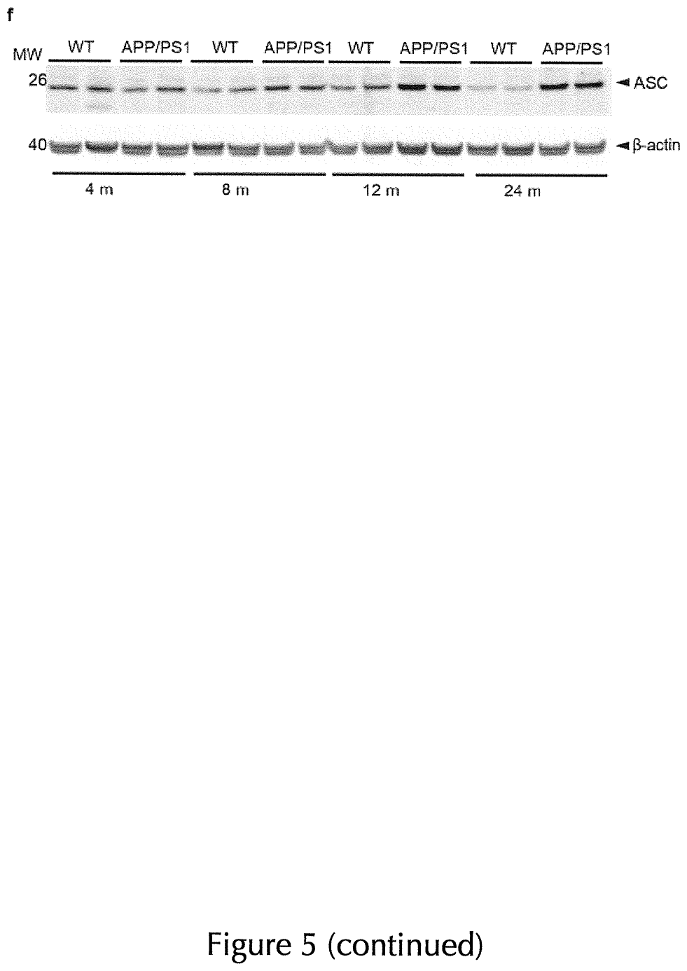

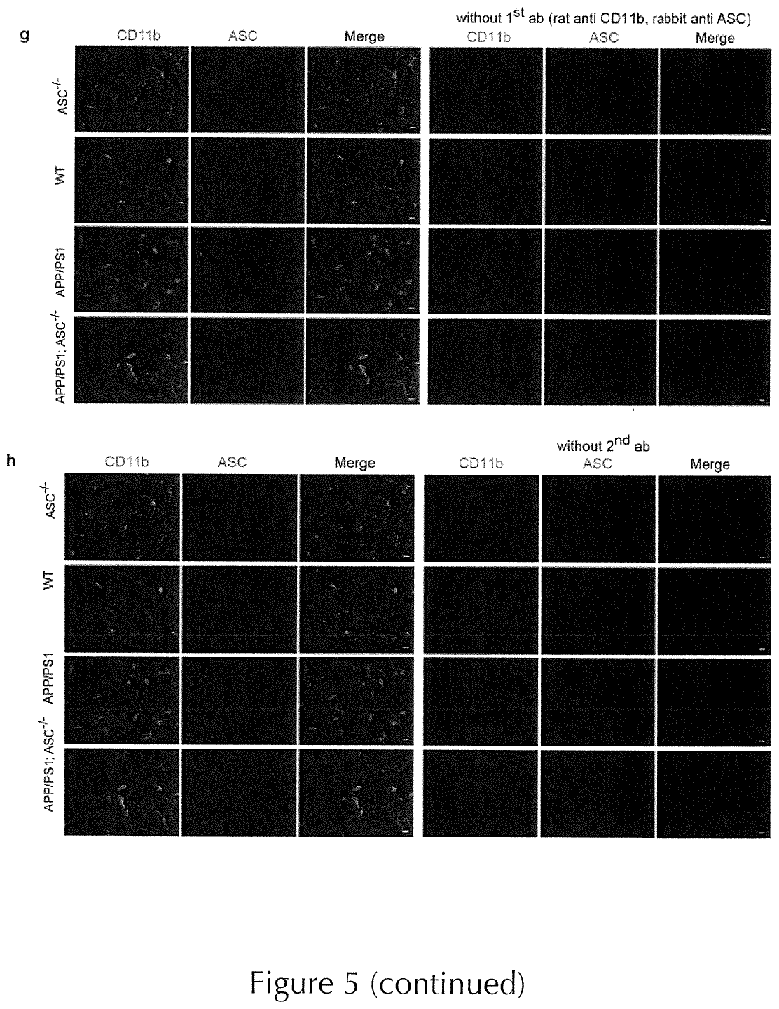

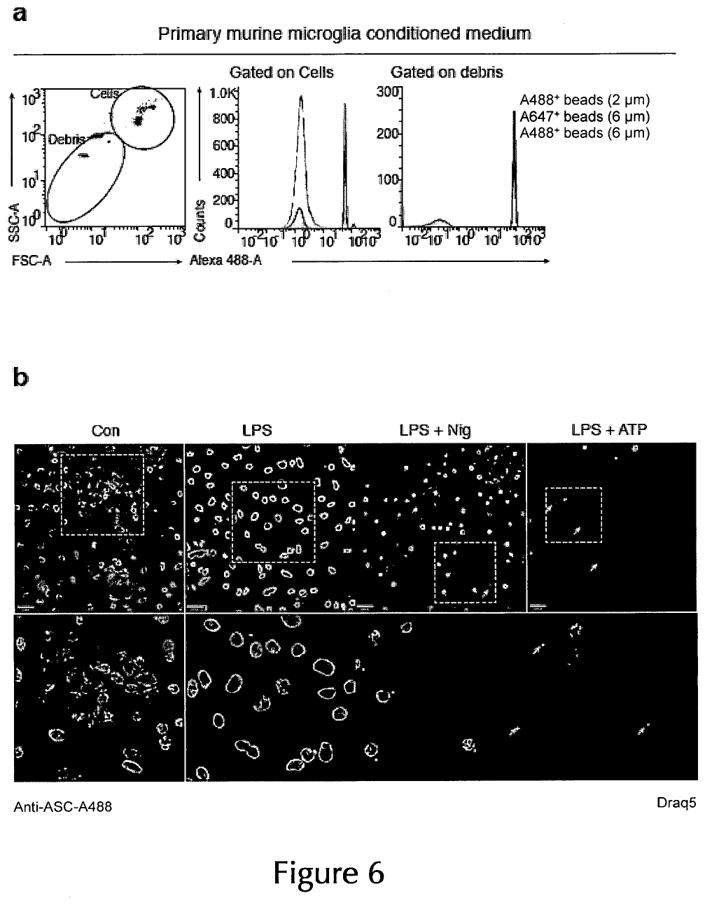

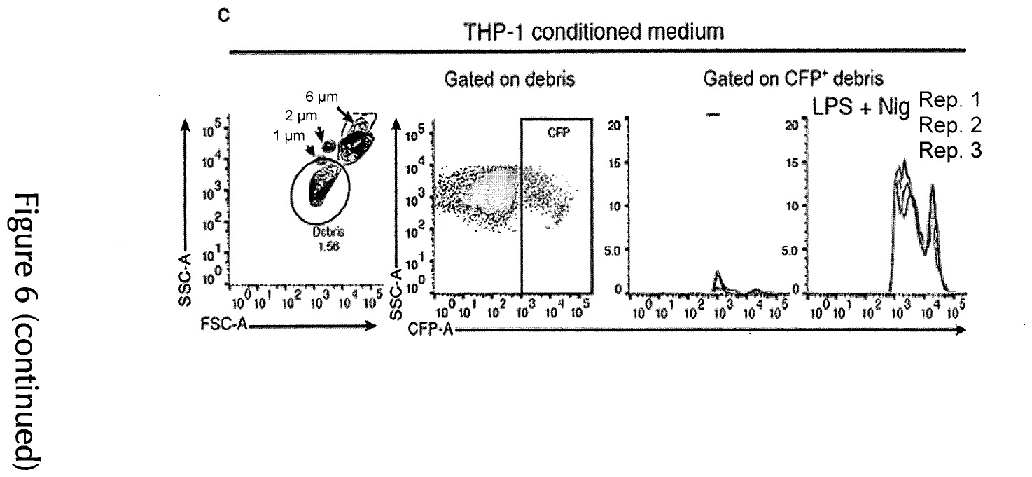

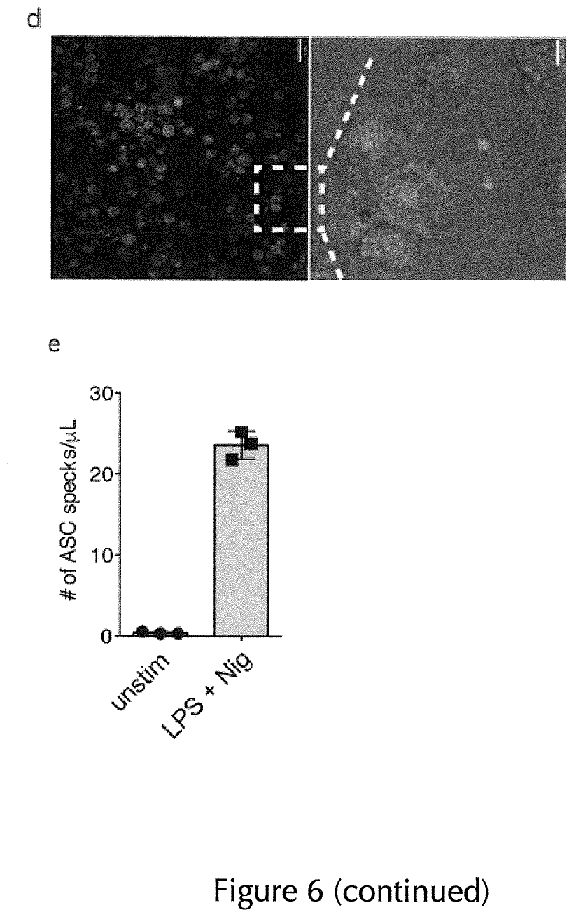

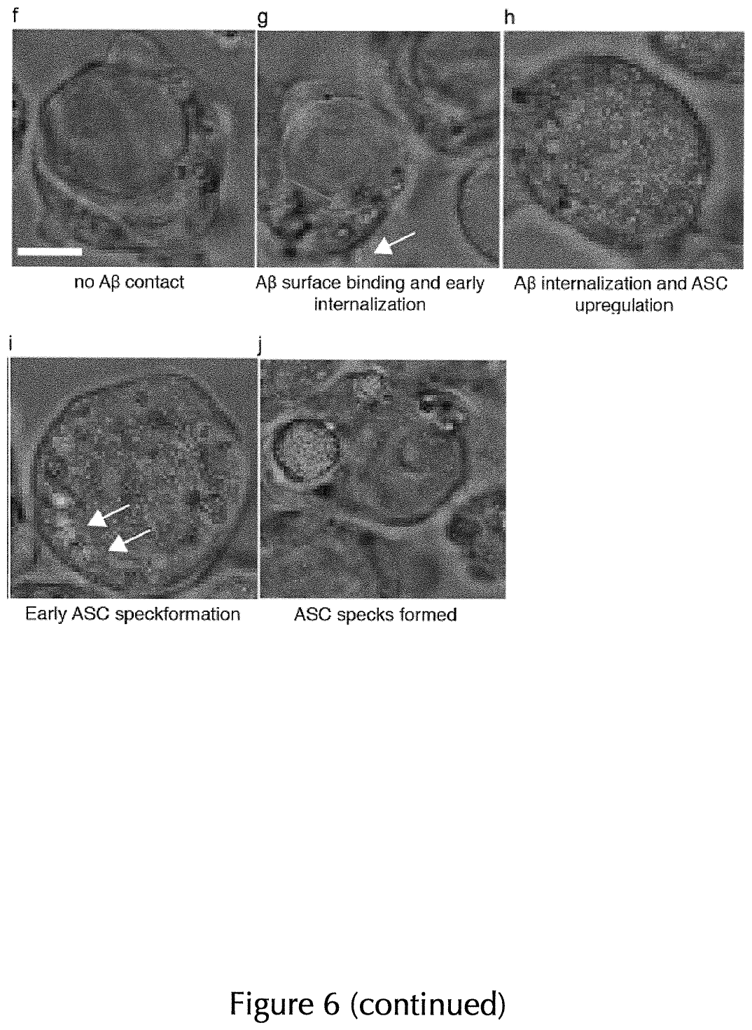

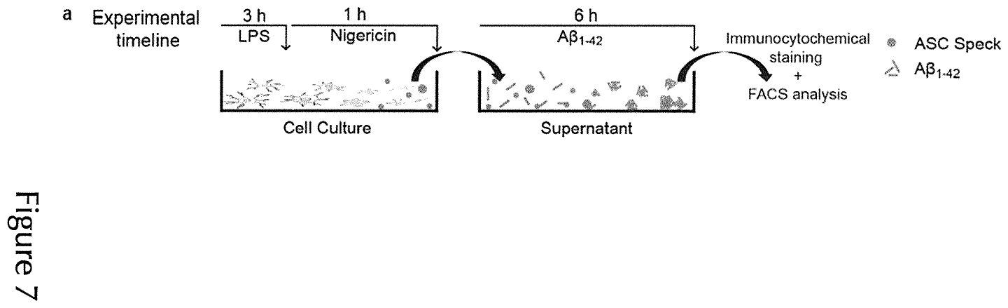

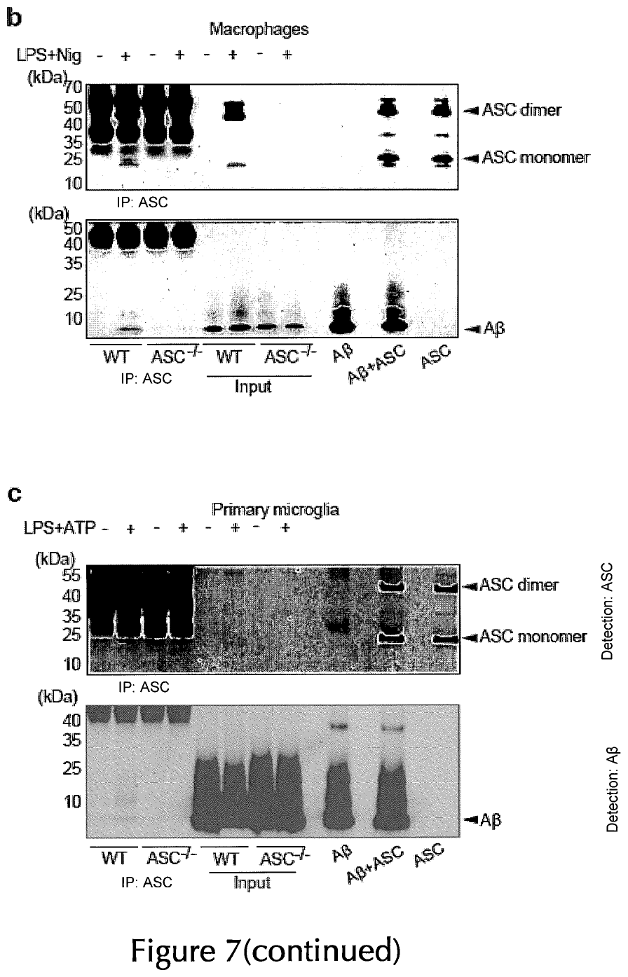

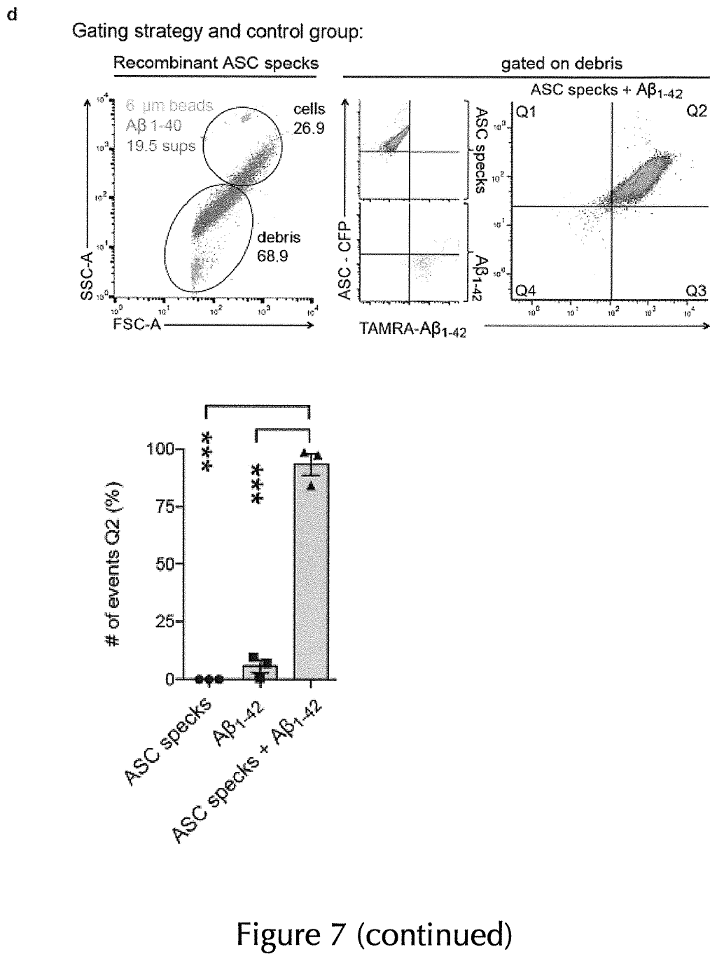

ASC specks can be visualized in brain sections of AD cases and APP/PS1 transgenic mice and are located within microglia and in the extracellular space and also bound to Aβ deposits (FIG. 1a,b, FIG. 5). In vitro, ASC speck formation and release can be induced in pre-stimulated murine microglia (FIG. 1c,d, FIG. 6a,b) or human THP-1 cells (FIG. 6c-j) by exposure to NLRP3 inflammasome activators. Exposure of microglia to Aβ₁₋₄₂ caused the formation and release of ASC specks. Dynamic imaging revealed that soon after their release, ASC specks bound to TAMRA-labelled Aβ₁₋₄₂ (FIG. 1e). This observation was further substantiated by incubation of supernatants derived from inflammasome-stimulated primary wild-type and ASC^{-/-} microglia (FIG. 1f-h) or macrophages with Aβ₁₋₄₂ and subsequent FACS analysis (FIG. 7a-e) or immunoprecipitation experiments (FIG. 7b,c). Supernatants from ASC^{-/-} microglia or macrophages failed to influence Aβ aggregation, which became only detectable in supernatants derived from ASC-producing cells (FIG. 1g,h, FIG. 4e,f).

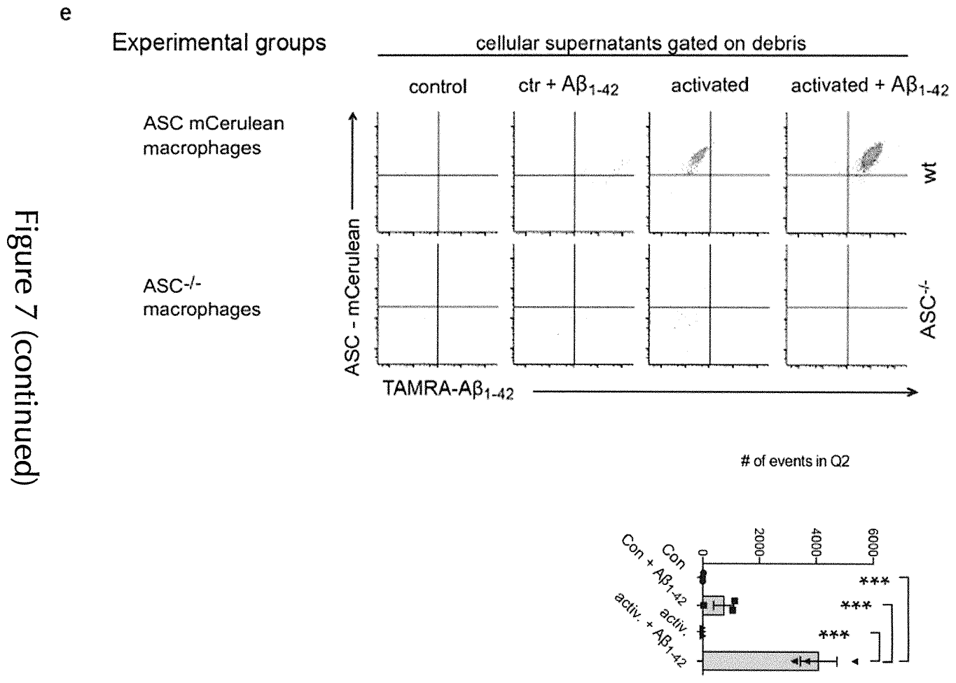

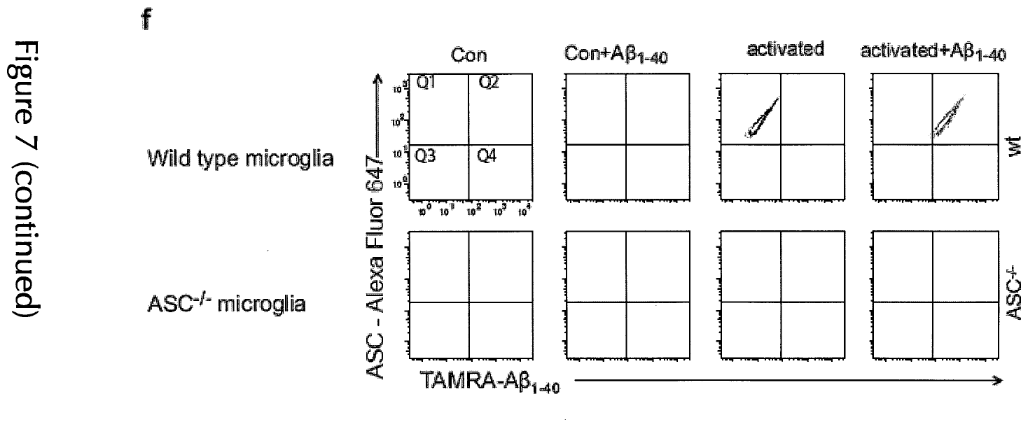

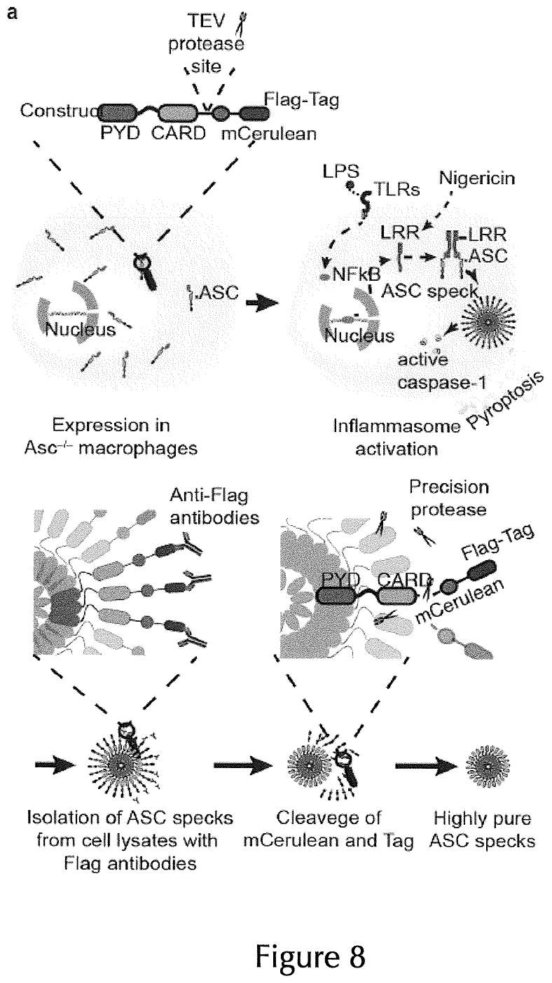

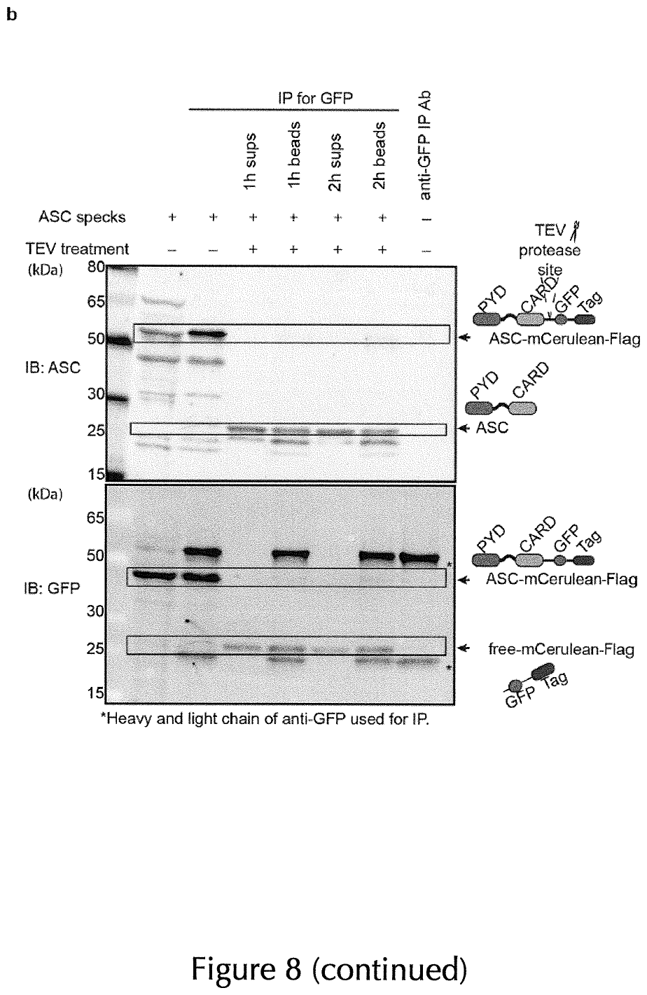

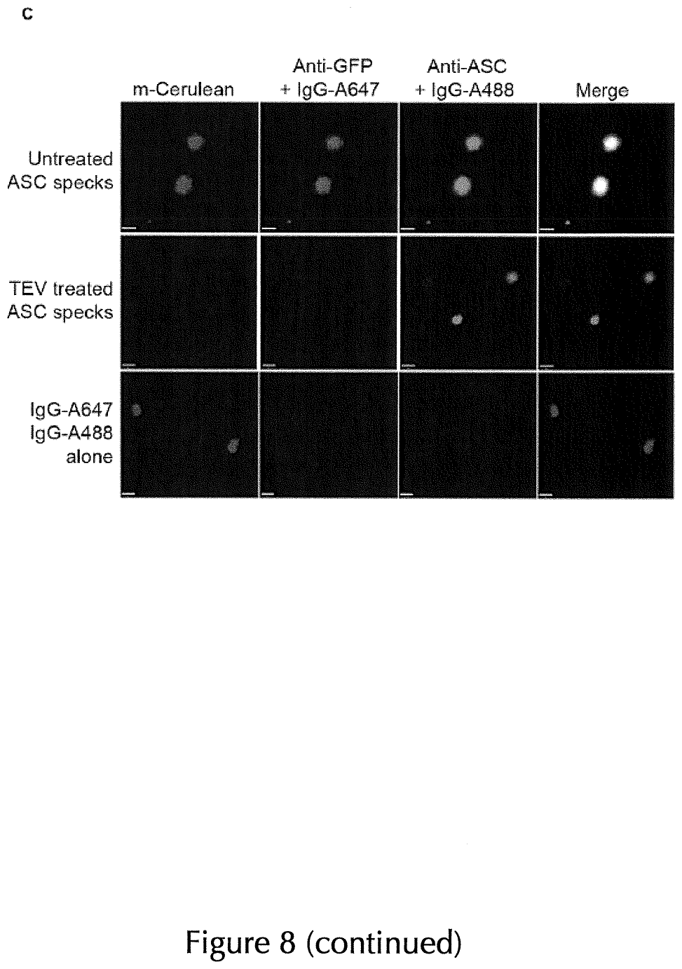

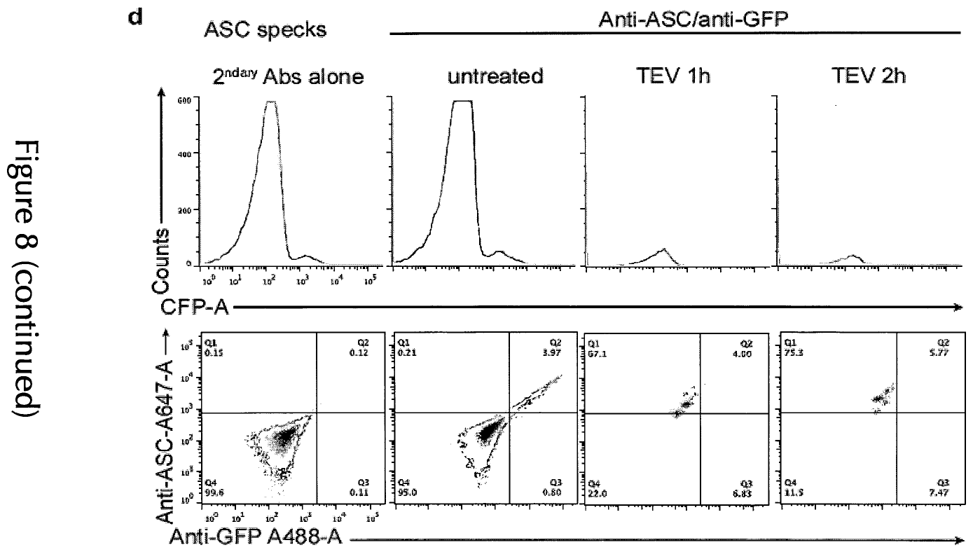

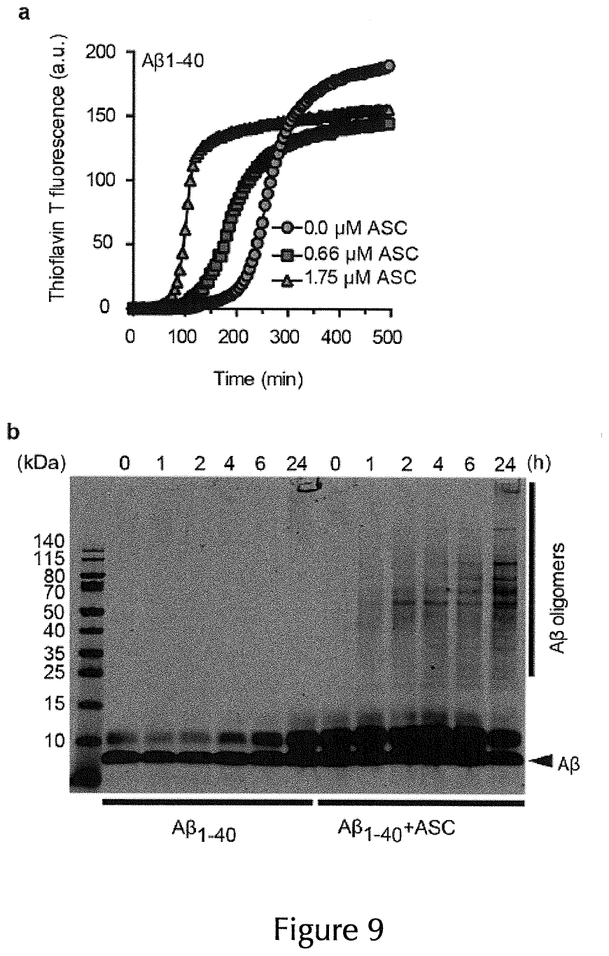

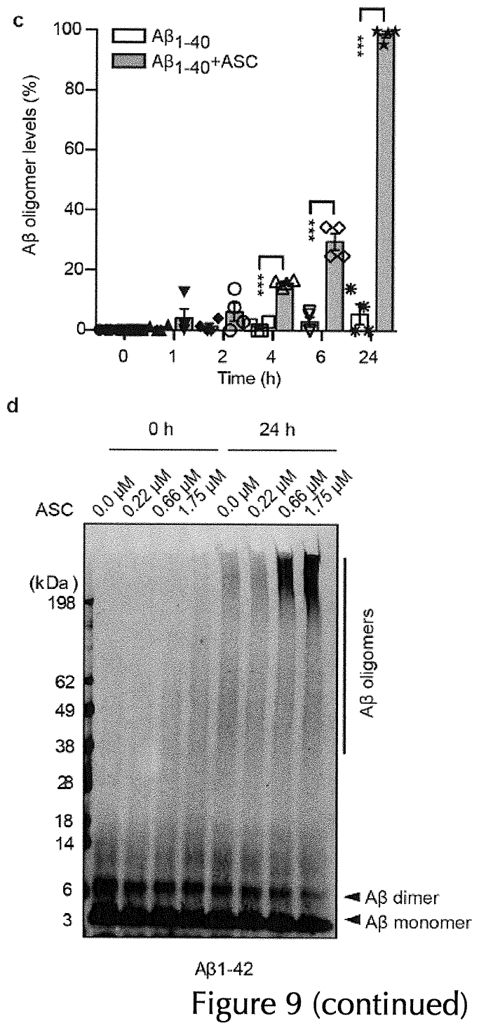

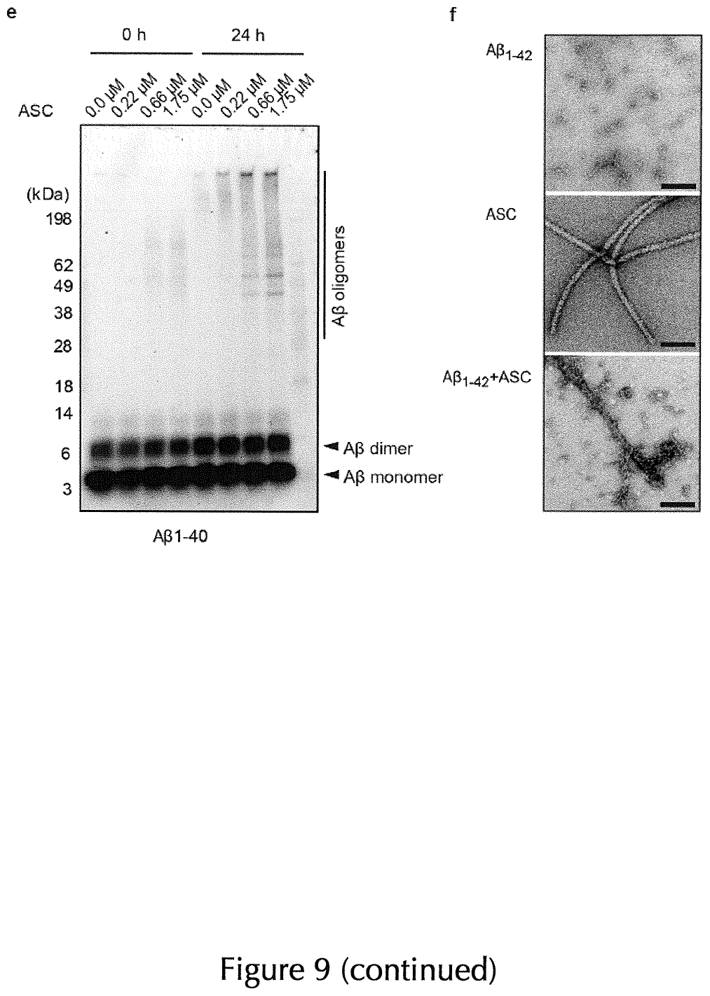

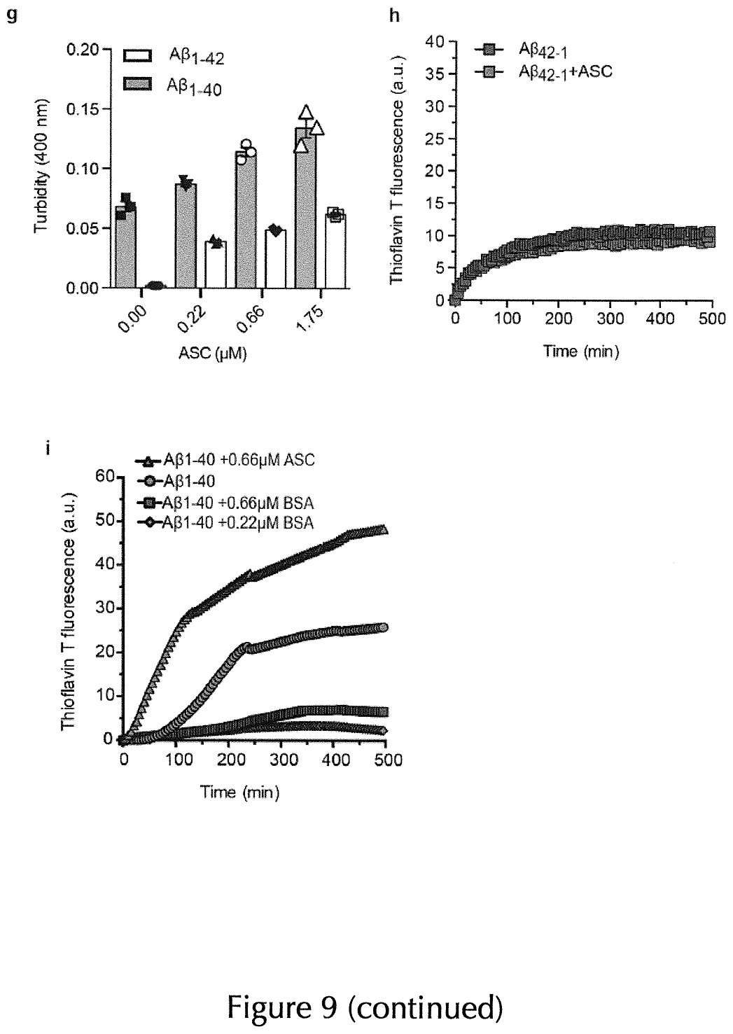

Thioflavin T fluorescence assay and Western blot analysis, using purified ASC specks generated by immunoprecipitation and enzymatic release (FIG. 8), further revealed that co-incubation with Aβ₁₋₄₂ (FIG. 1i-k, FIG. 9d) or Aβ₁₋₄₀ (FIG. 9a-c,e) accelerated Aβ aggregation in a time- and concentration-dependent manner. Here, the decreased lag phase of aggregation in the presence of ASC indicates an enhanced formation of seeding nuclei through the interaction of two different peptides/proteins, and thus a cross-seeding activity of ASC specks for Aβ aggregation (FIG. 1i, FIG. 9d,e). These results were further corroborated by turbidity assay measurements and transmission electron microscopy (FIG. 9f,g). Notably, control experiments showed that ASC specks did not induce the aggregation of the reverse sequence of Aβ₁₋₄₂ nor a control peptide (bovine serum albumin) (FIG. 9h,i).

### Example 2: Aβ Cross-Seeding Depends on ASC-PYD Domain

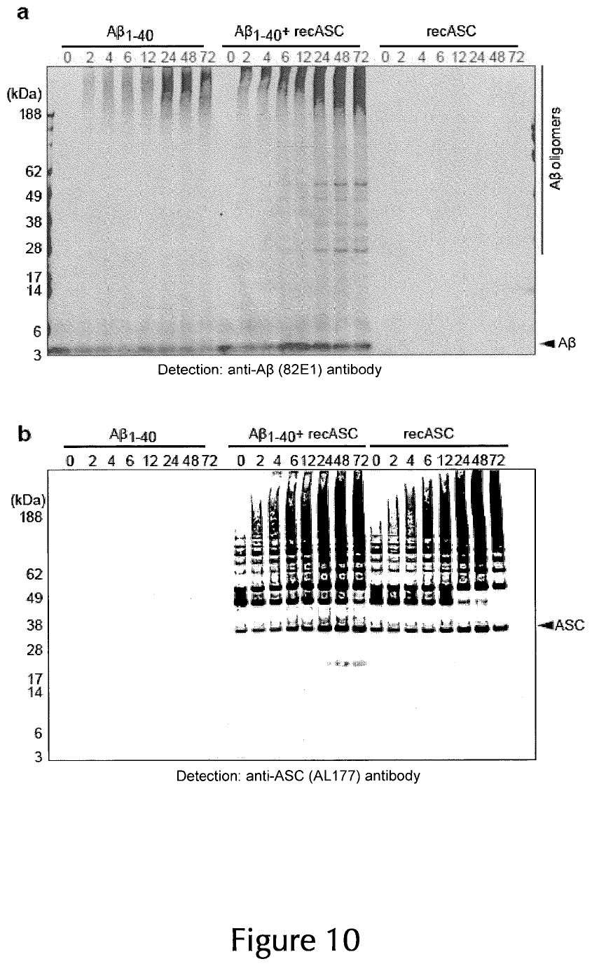

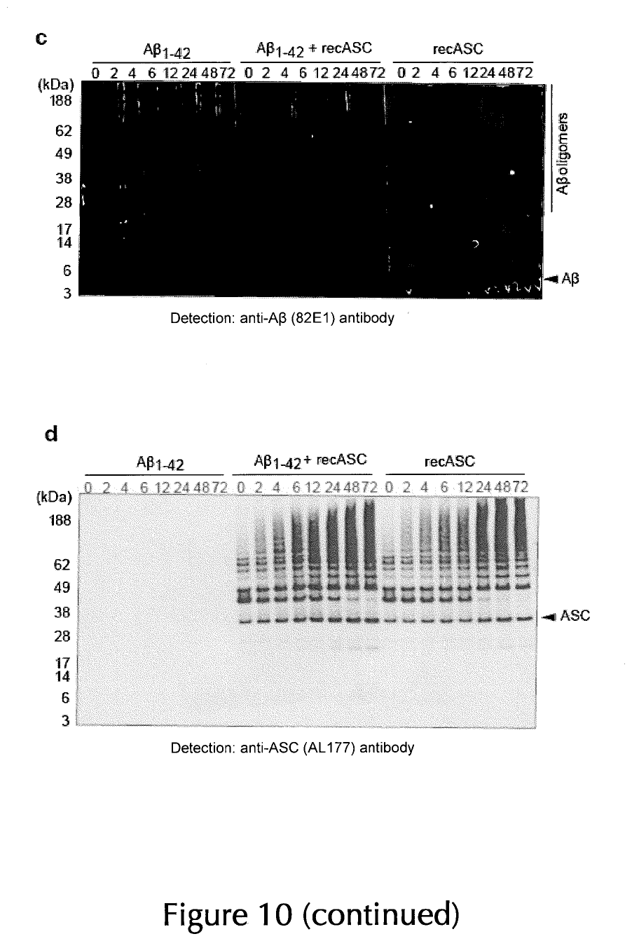

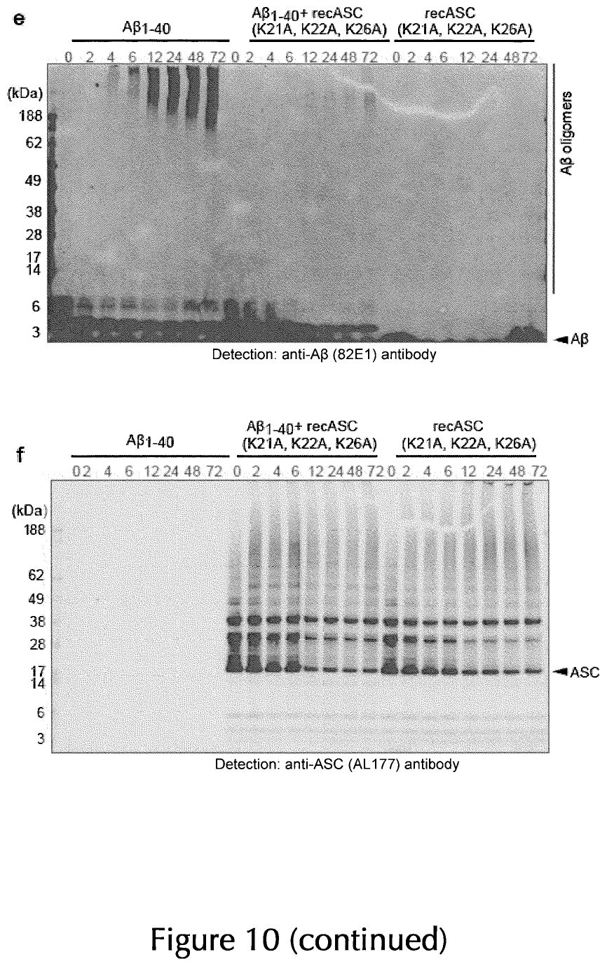

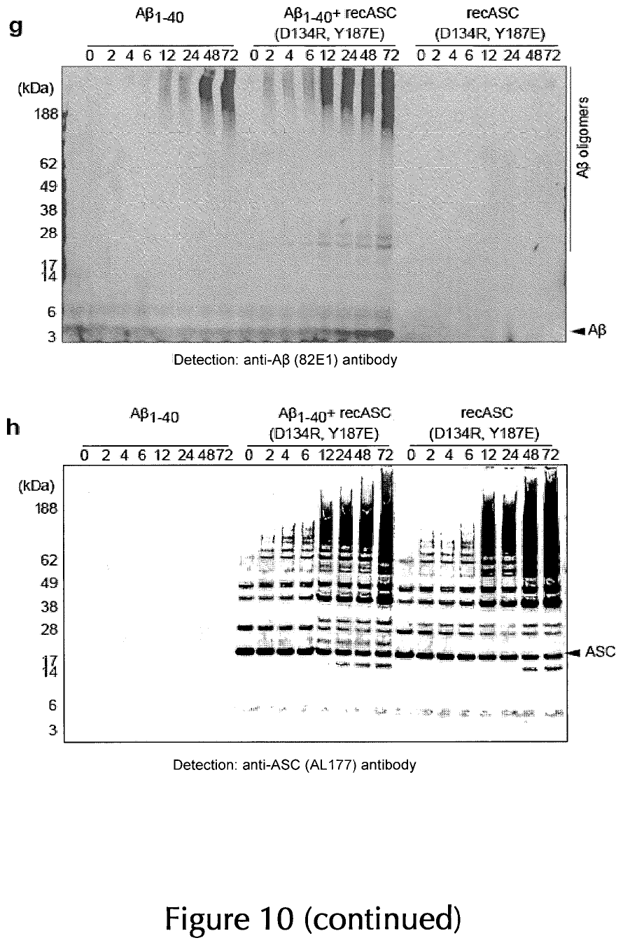

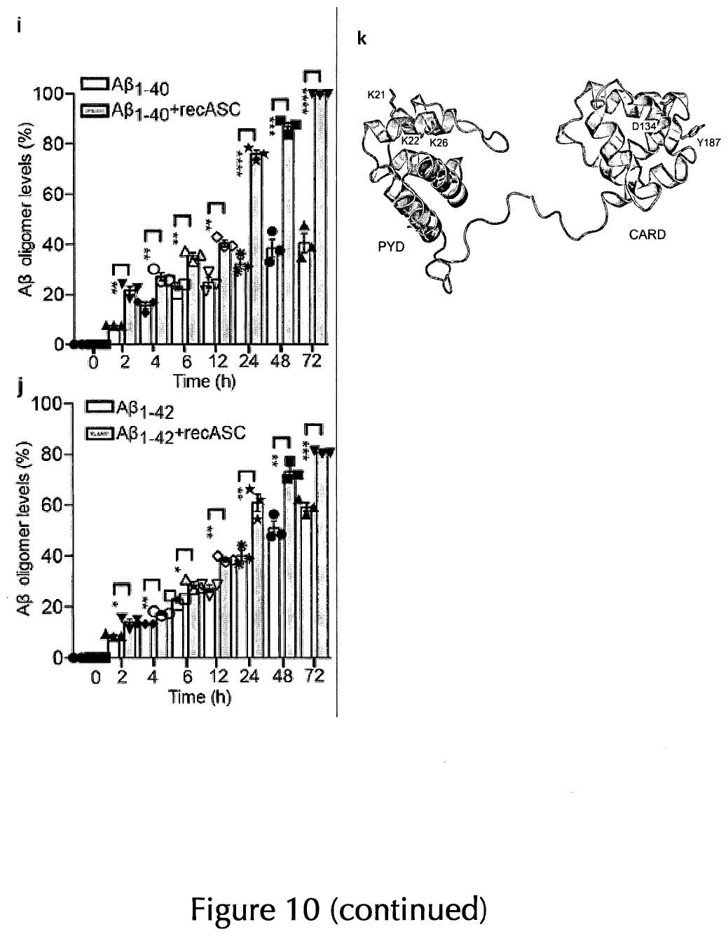

ASC specks derived from recombinant protein (recASC) likewise promoted Aβ₁₋₄₀ and Aβ₁₋₄₂ aggregation from early time points on, as detected by immunoblotting experiments (FIG. 10a-d, i, j) confirming the previous observations. To further support the specific interaction of ASC specks and Aβ, recASC carrying mutations either located in the PYD or CARD domain of ASC were tested. Mutations of the ASC-PYD domain at position 21, 22, and 26, which prevent ASC helical fibril assembly¹⁸, completely prevented the ASC speck promoting effect on Aβ aggregation (FIG. 10e). In contrast, point mutations in the ASC CARD domain, which prevent ASC fibril self-assembly which aids in ASC speck formation, did not substantially change ASC-speck mediated promotion of Aβ aggregation (FIG. 10f,g). The aggregation propelling action of ASC is reminiscent of several fAD causing mutations in genes coding for APP or presenilin, which increase the aggregation propensity of the Aβ peptide¹⁹⁻²¹. In particular, given the effect of ASC on Aβ₁₋₄₀ aggregation, microglial innate immune responses may accomplish a similar effect through ASC speck release. One may therefore speculate whether factors that increase the risk for sAD and are also known to involve inflammasome activation in the brain act through this mechanism²².

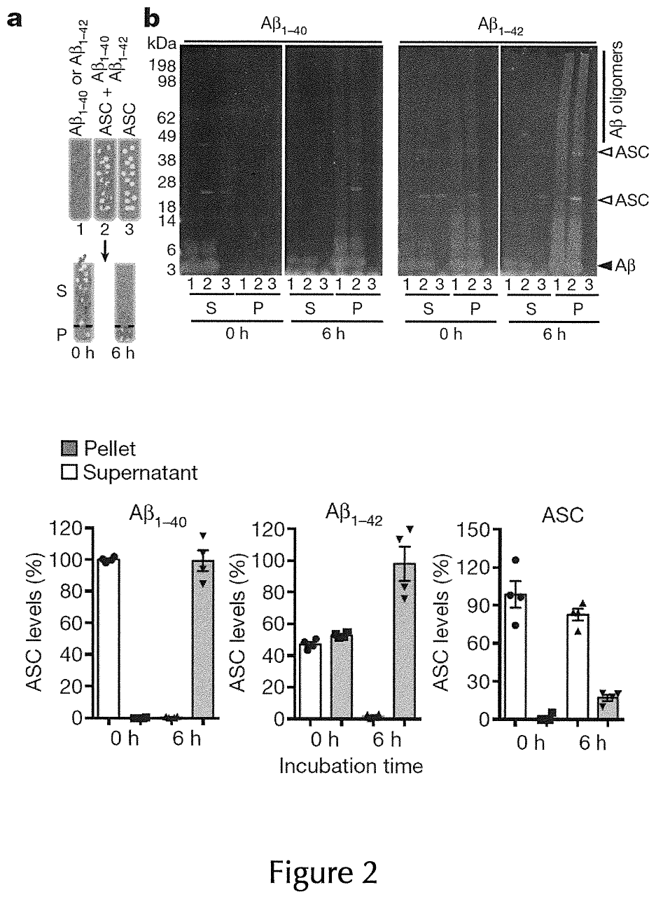

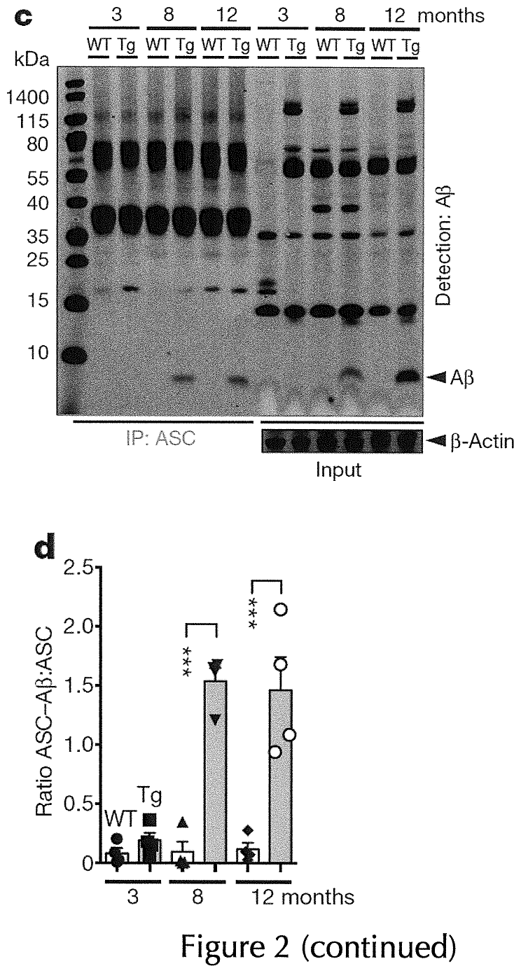

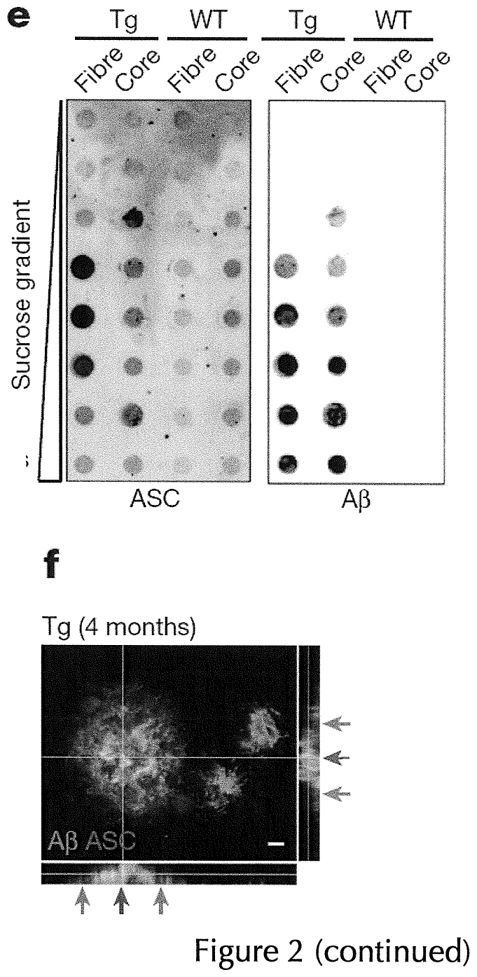

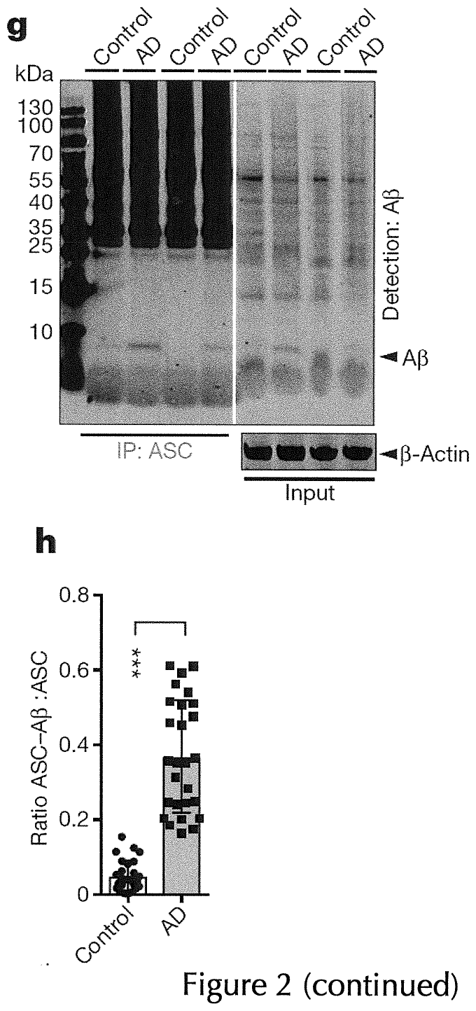

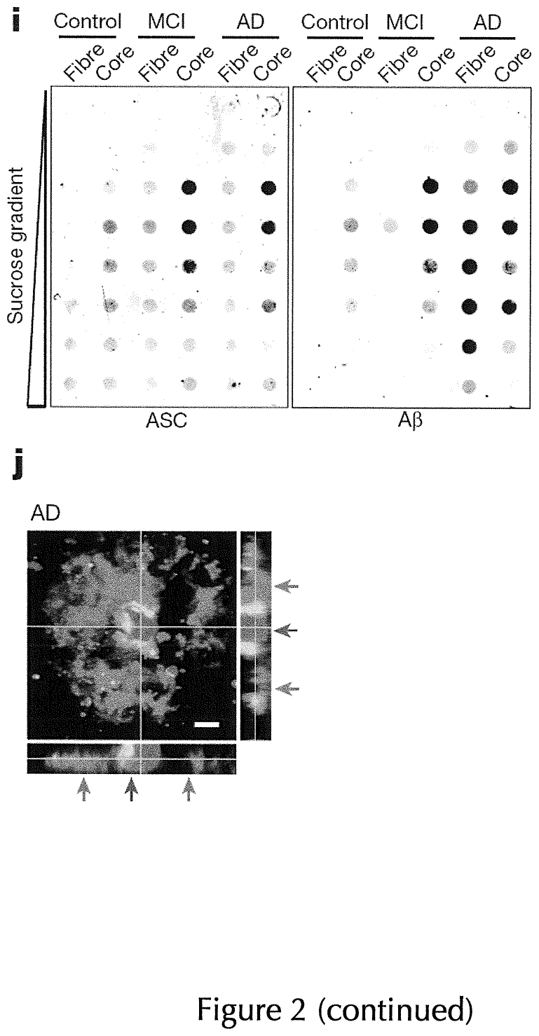

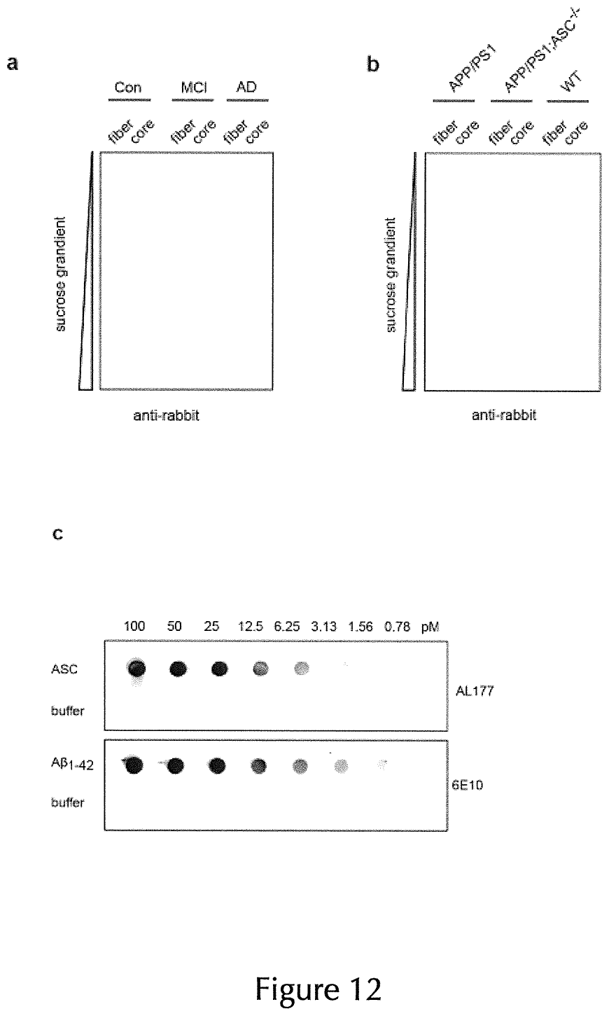

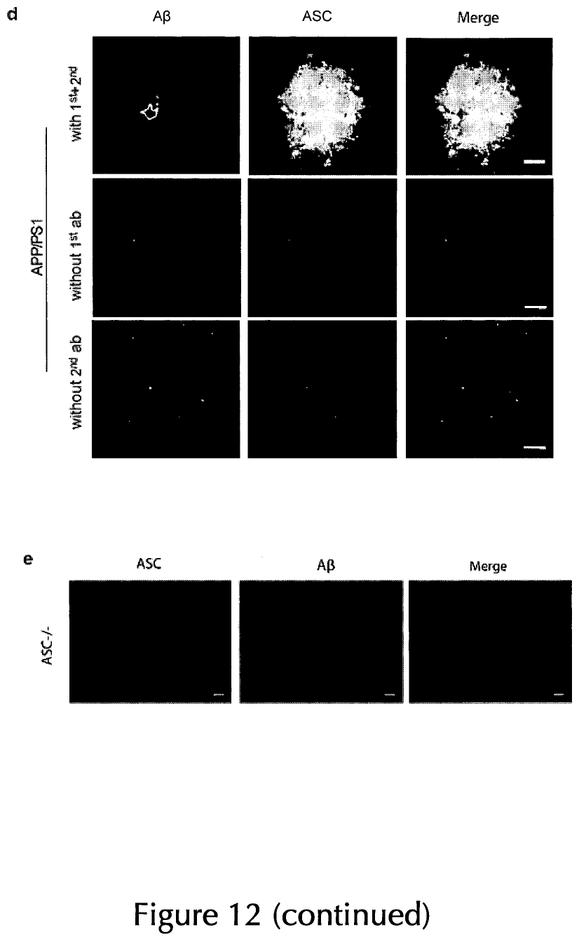

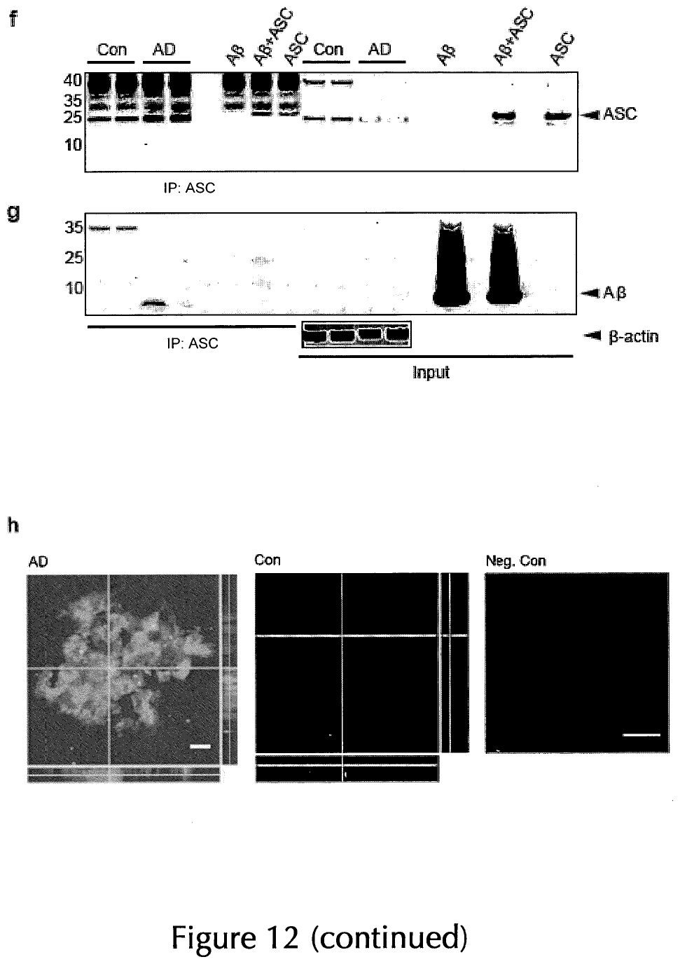

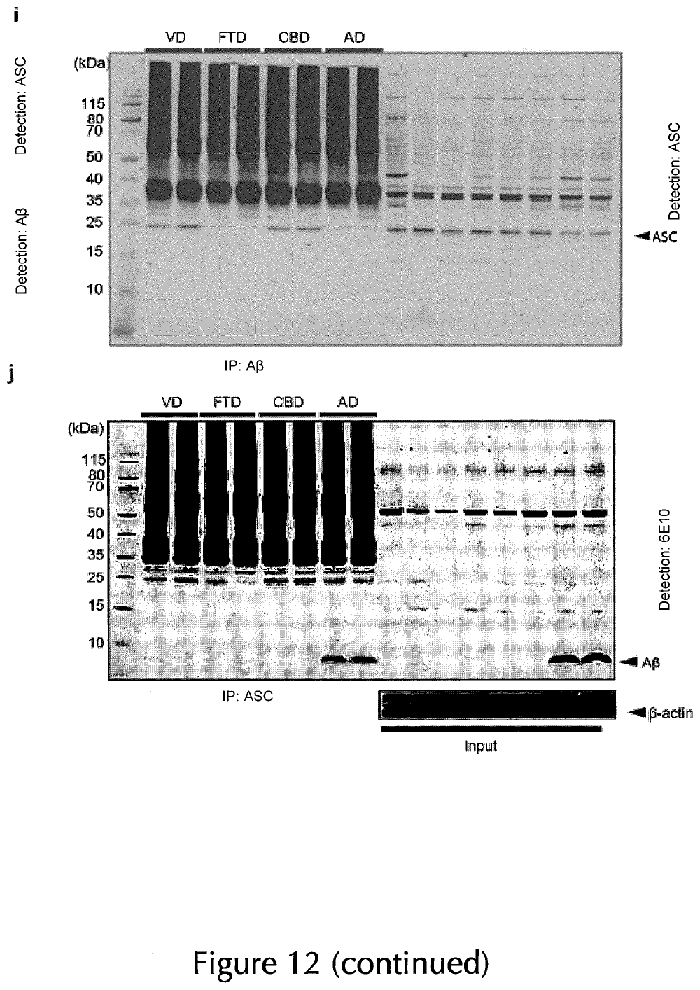

To further determine the physical interaction of ASC specks and Aβ, co-sedimentation assays were performed. ASC specks co-sediment in the pellet fraction within 6 h of incubation only in the presence of Aβ₁₋₄₀ and Aβ₁₋₄₂ but remained in the supernatant fraction at all time points in the absence of Aβ₁₋₄₀ and Aβ₁₋₄₂ peptide (FIG. 2a,b). Additional thioflavin T experiments on the supernatant and pellet fractions of the co-sedimentation assay samples demonstrated increased beta-sheet rich oligomer and fibrils in the presence of ASC (FIG. 11). Consistent with the ASC-Aβ interaction observed in the co-sedimentation experiments, ASC and Aβ co-immunoprecipitated from brain samples of APP/PS1 mice (FIG. 2c,d). Aβ binding to ASC increased with age and was absent in wild-type animals. Compartmental analysis of Aβ deposits isolated from the APP/PS1 brain by gradient centrifugation revealed the presence of ASC along with A1 in the core fraction, but also in the fiber fraction (FIG. 2e, FIG. 12a-c). In line with this, immunohistochemistry revealed that even the early Aβ deposits at 4 months of age show an ASC-immunopositive core, which is surrounded by antibody 6E10-immunopositive Aβ (FIG. 2f, FIG. 12d,e). This suggests that ASC speck-mediated innate immune responses may result in cross-seeding of Aβ at an early stage of Aβ aggregation and deposition in vivo. Similarly, ASC-bound Aβ was nearly absent in human brain samples from non-demented age-matched controls, but strongly increased in AD brains (FIG. 2g,h, FIG. 12f,g). Analysis of core and fiber compartments of Aβ deposits found that, in contrast to controls, patients suffering from mild cognitive impairment (MCI) due to AD, a clinical pre-phase of overt AD dementia, had ASC-Aβ co-localization in the core fractions, while the fiber fractions showed only minor immunoreactivity for both targets (FIG. 2i). Similarly, AD was characterized by the co-presentation of ASC and Aβ within the core, while the fiber fractions remained mainly immunopositive for Aβ, suggesting that ASC speck-Aβ cross-seeding occurs prior or during MCI (FIG. 2i), causing ASC immunostaining of the core surrounded by Aβ (FIG. 2j, FIG. 12h). Notably, ASC-bound Aβ was undetectable in post-mortem tissue of patients suffering from other neurodegenerative diseases including fronto-temporal dementia, cortico-basal degeneration and vascular dementia (FIG. 12i,j).

### Example 3: ASC Specks Promote aβ Deposition In Vivo

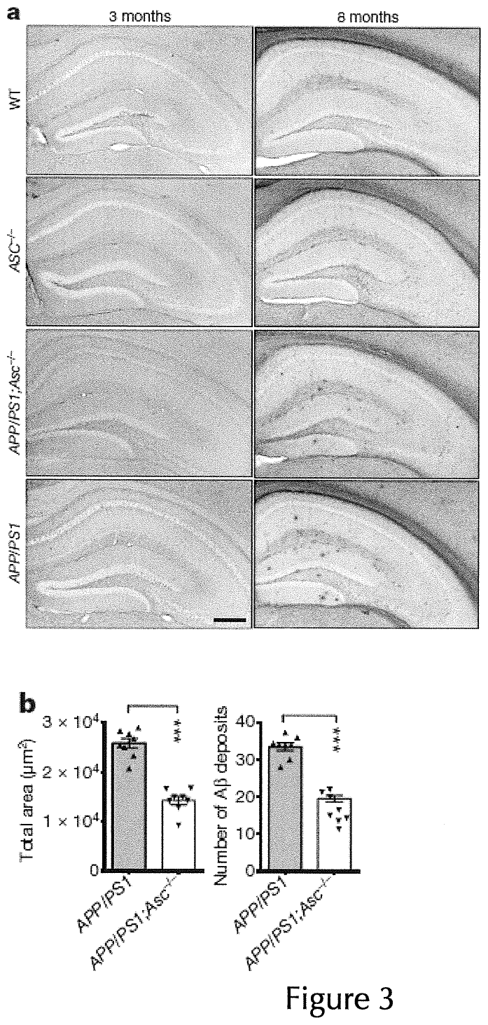

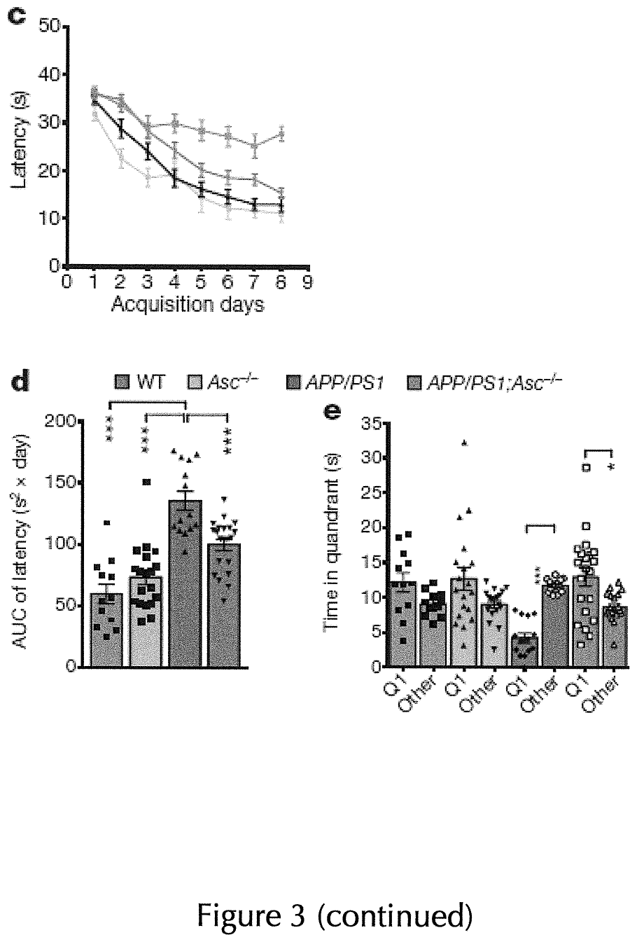

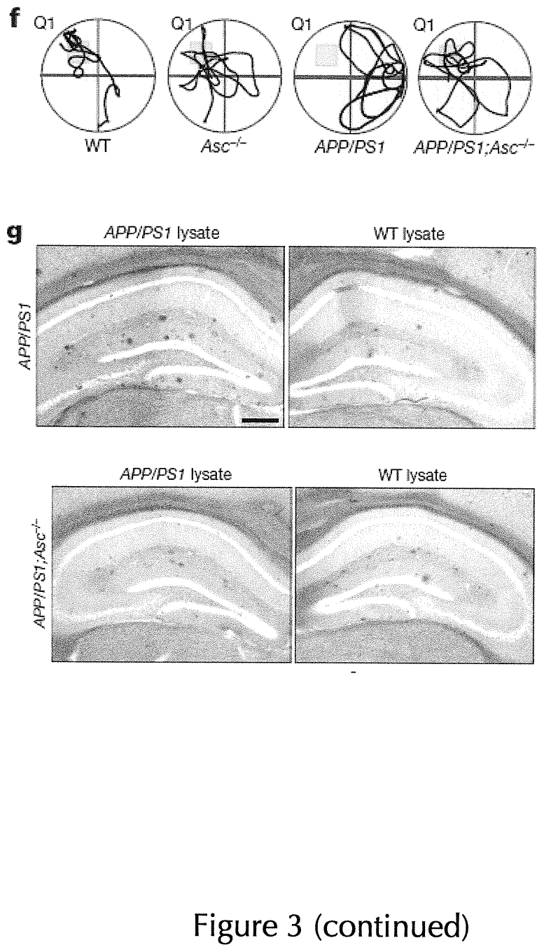

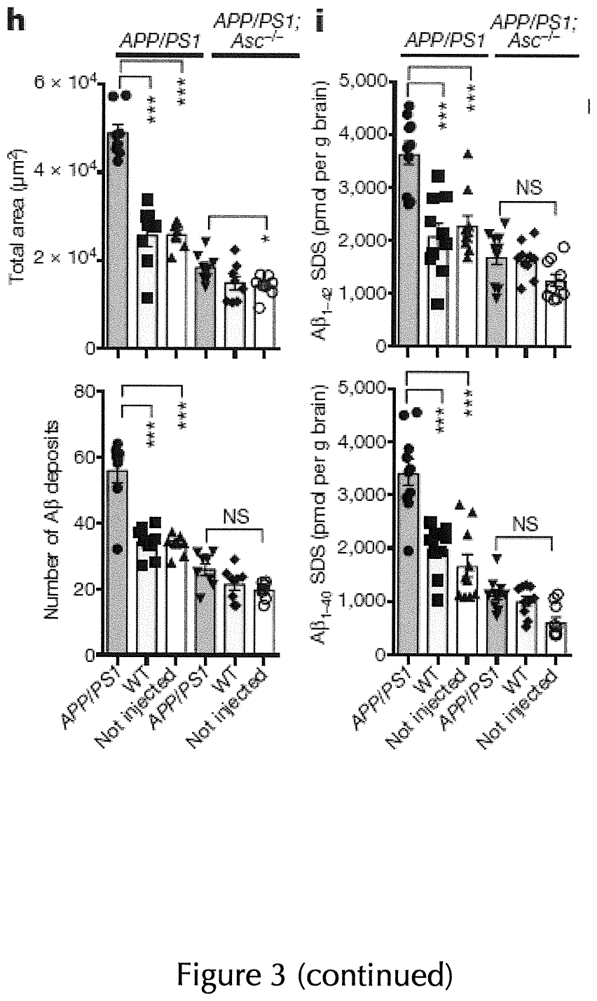

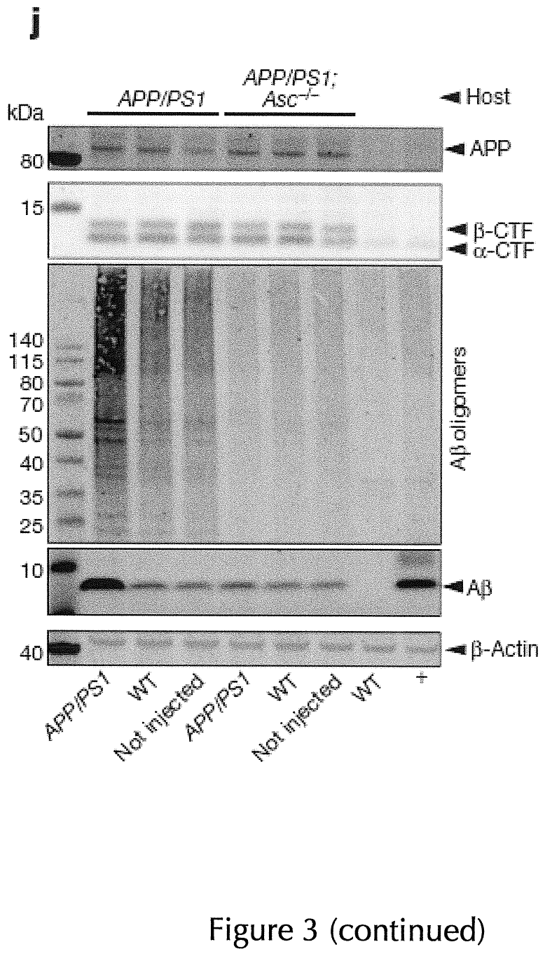

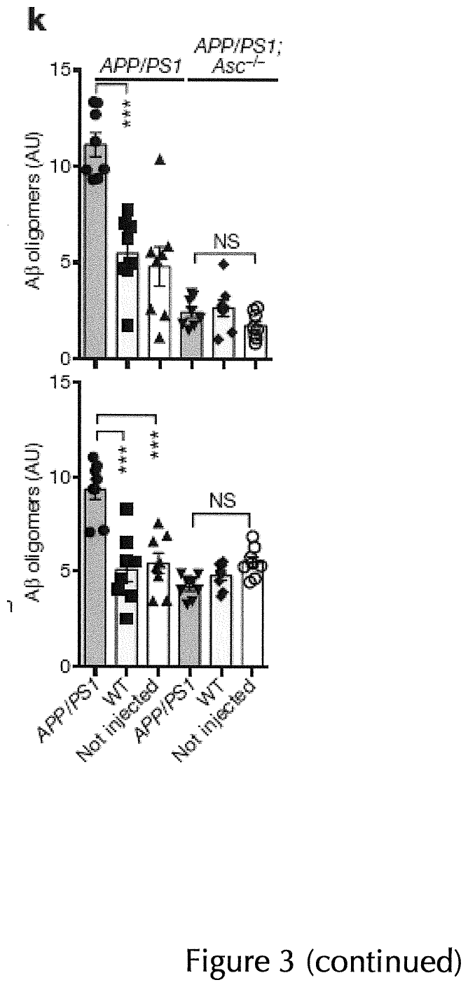

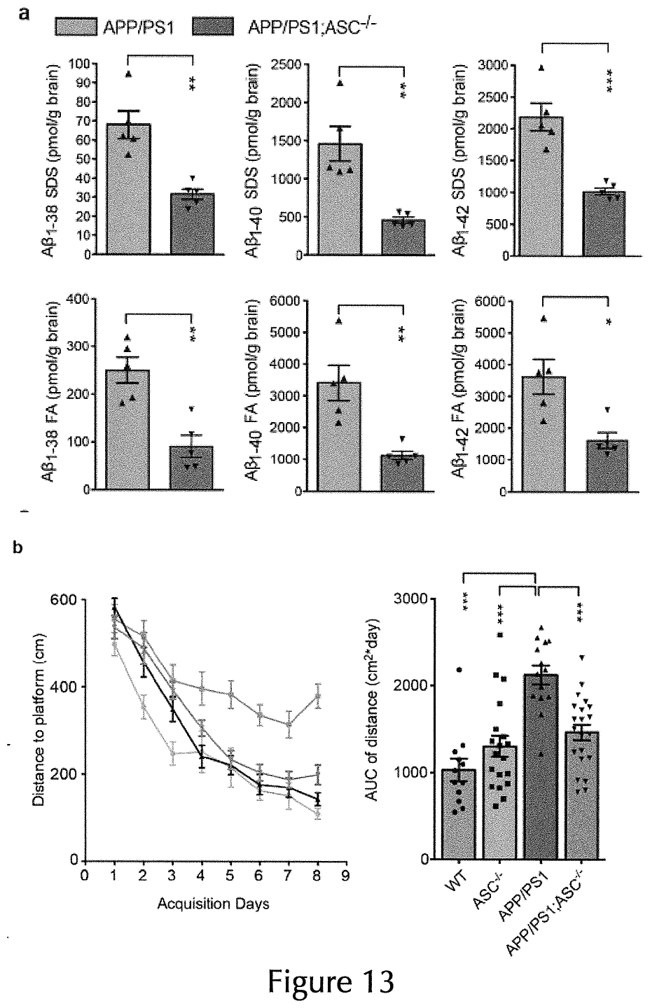

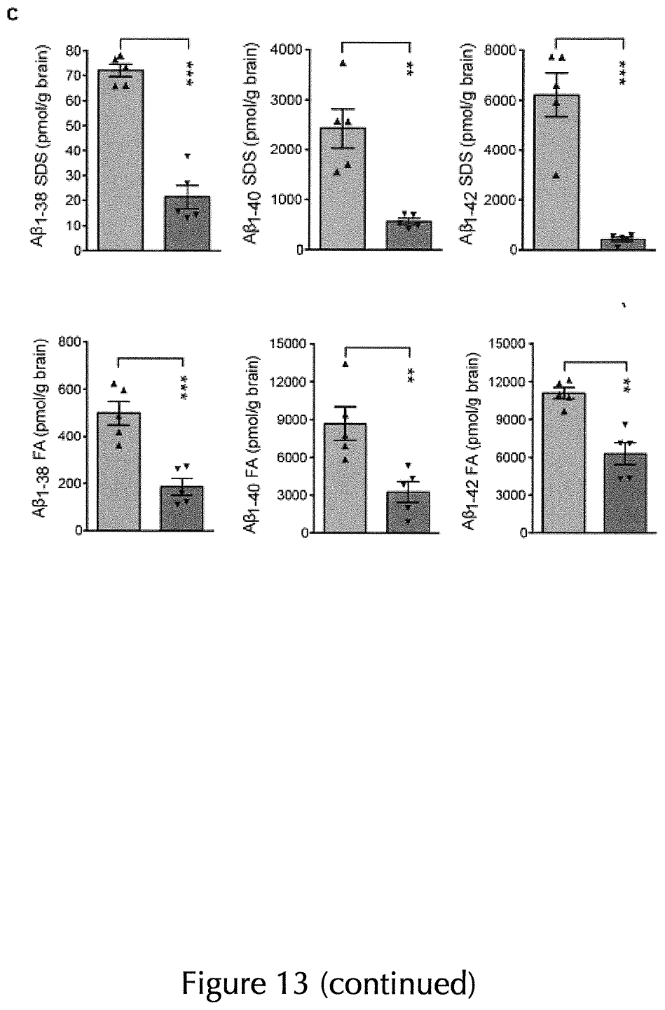

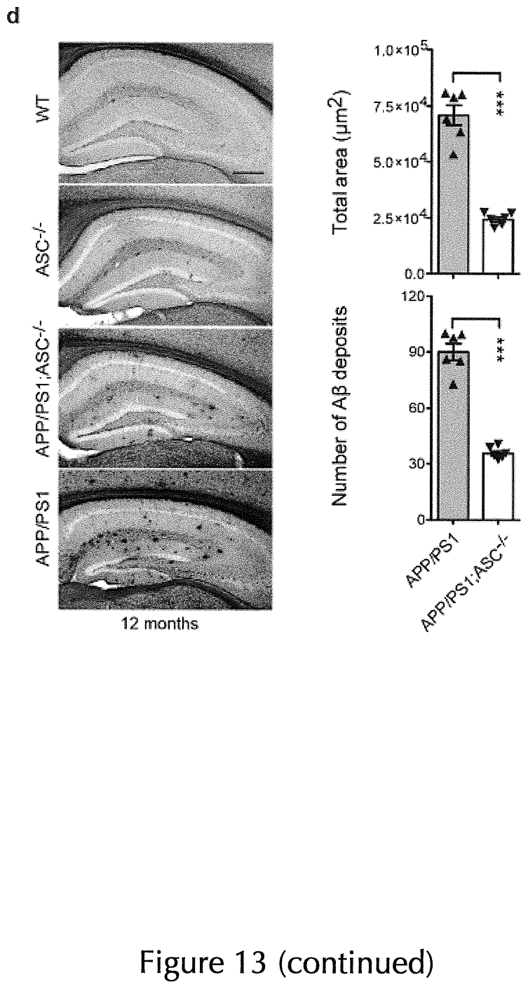

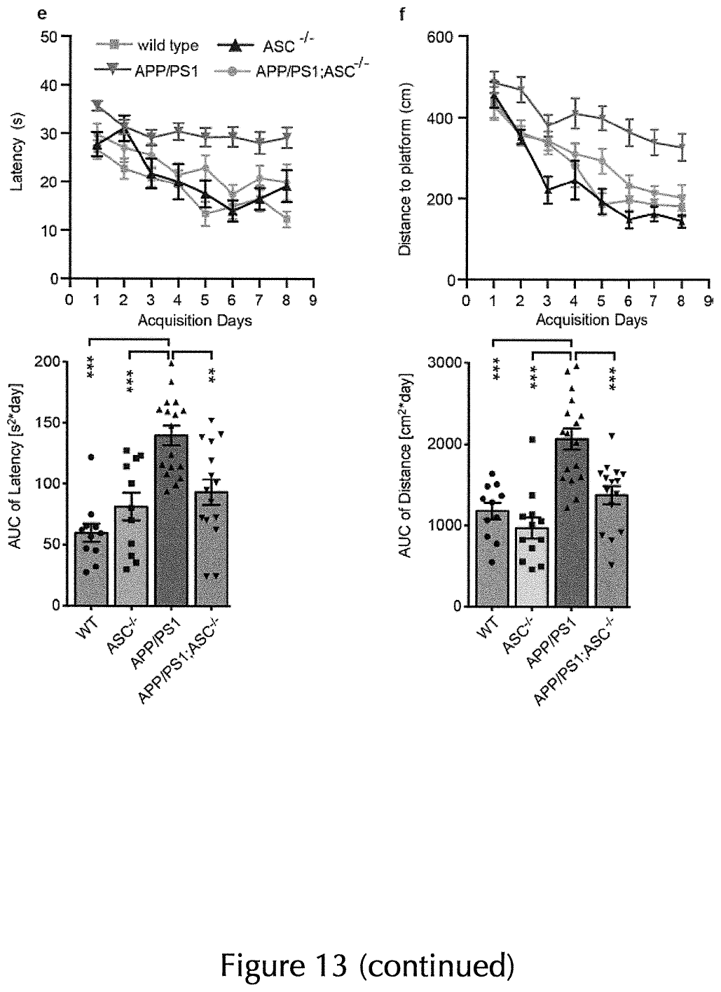

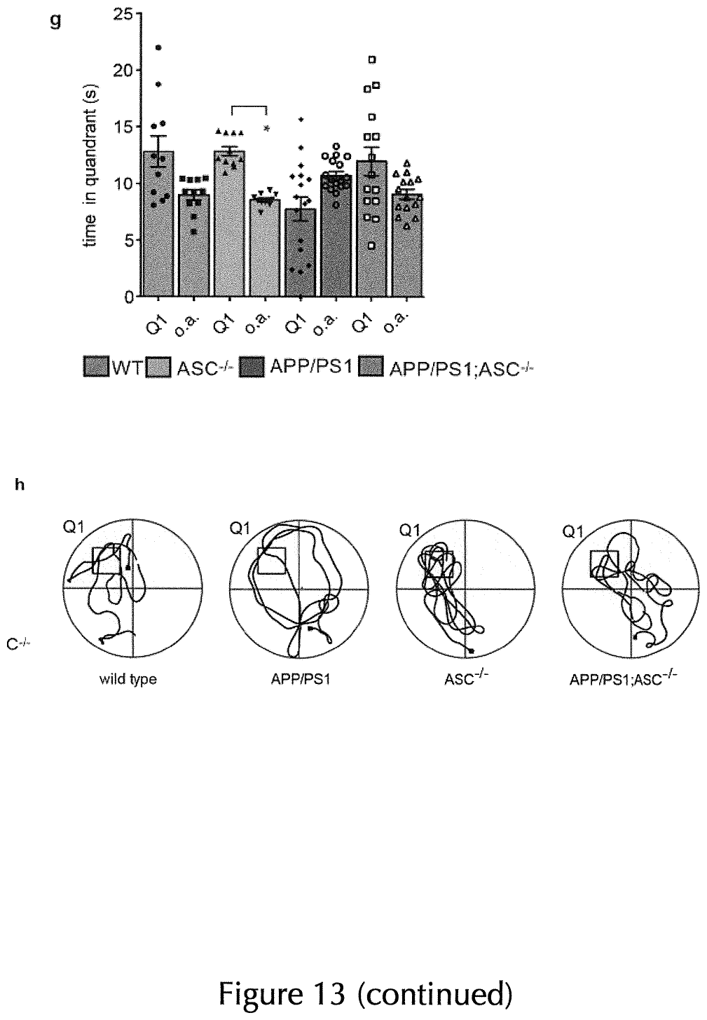

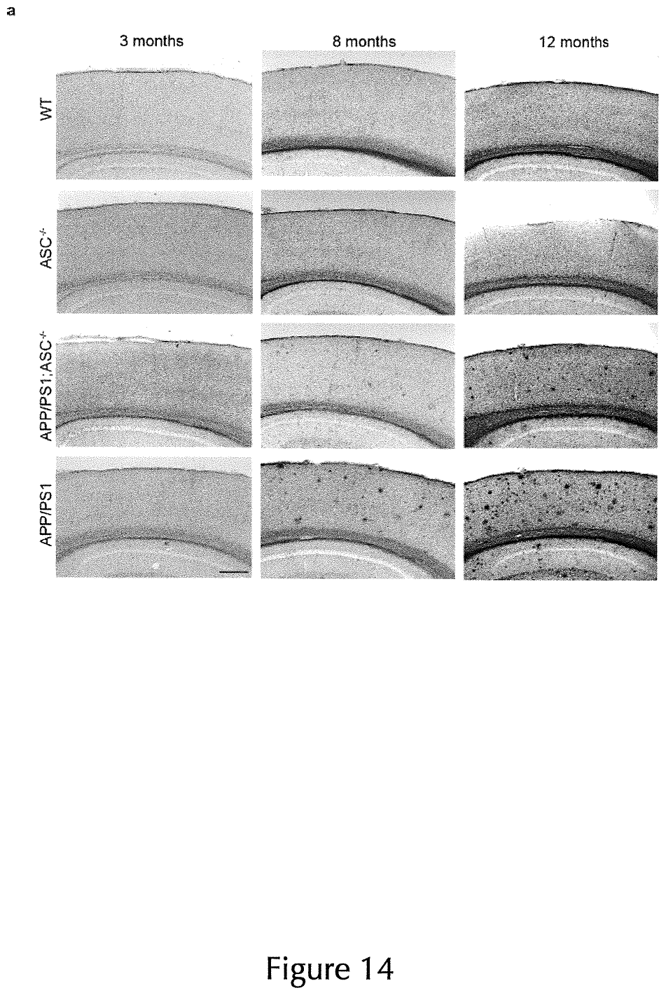

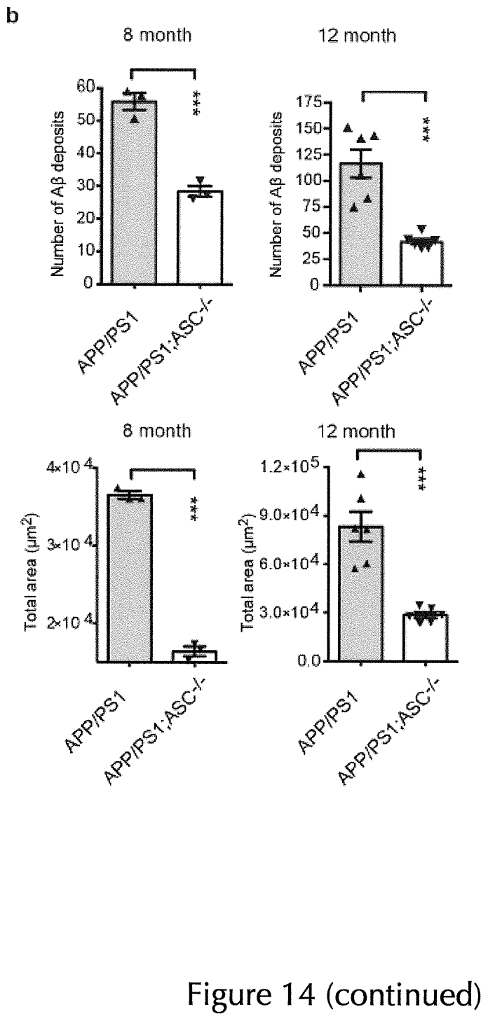

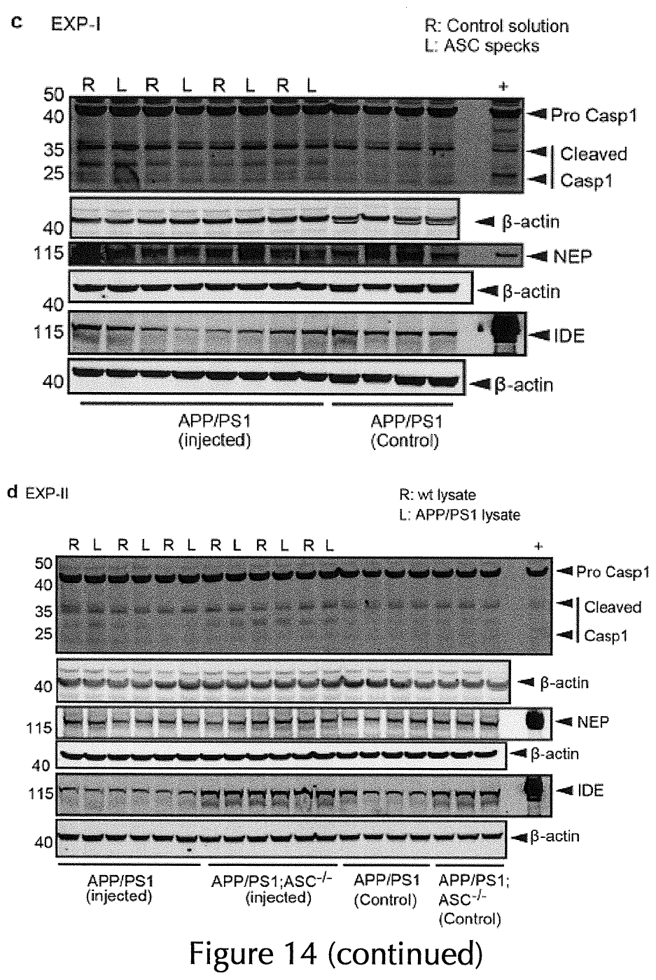

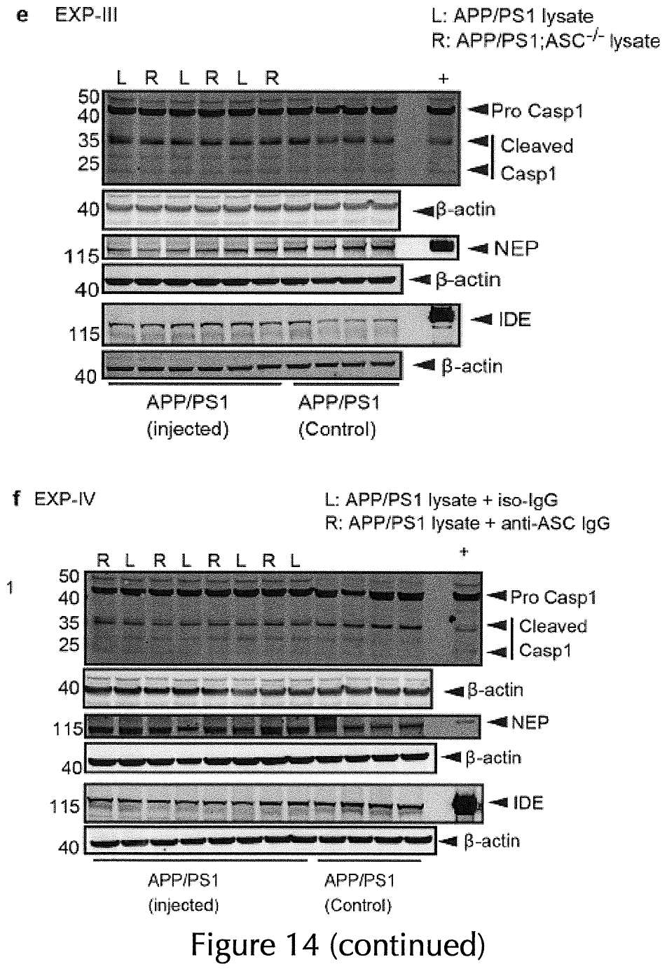

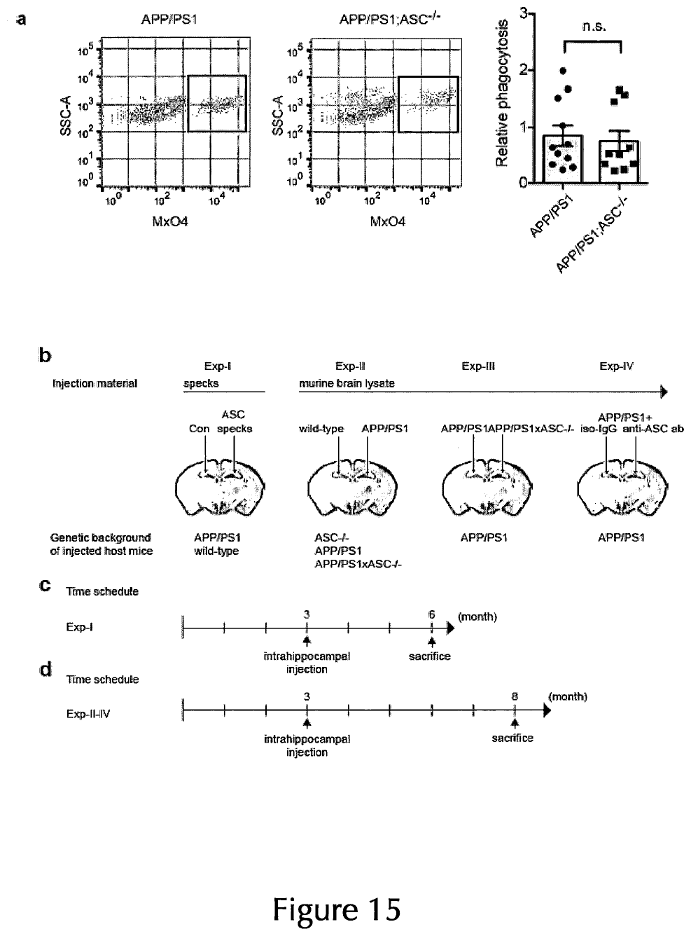

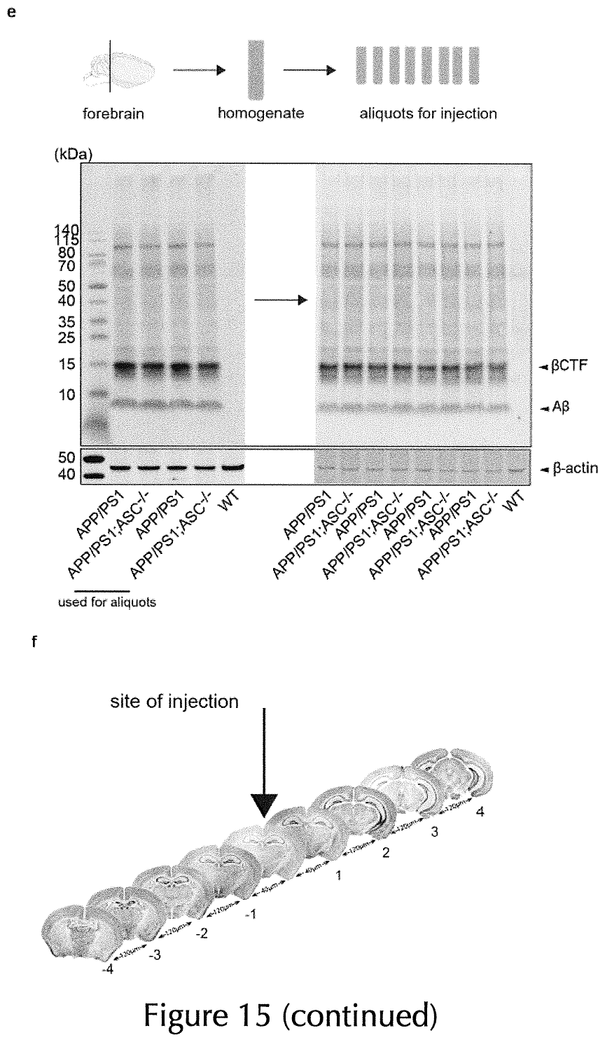

To characterize the overall impact of ASC on Aβ pathology and associated behavioural deficits, ASC knockout animals (ASC^{-/-}) were crossed to APP/PS1 transgenic mice and analyzed at 3, 8 or 12 months of age. While no differences were detectable at 3 months, APP/PS1/ASC transgenic mice had a significant reduction of cerebral Aβ load at 8 and 12 months (FIG. 3a,b, FIG. 13a,c,d, FIG. 14a,b). Of note, modulation of NLRP3-mediated immune mechanisms, previously described in aged 16-month old APP/PS1 transgenic mice, including caspase-1 activation (CASP1, FIG. 14c-f), generation of Aβ degrading enzymes neprilysin (NEP, FIG. 14c-f) and insulin-degrading enzyme (IDE, FIG. 14c-f) or phagocytosis (FIG. 15a) did not account for the observed changes in cerebral Aβ. Likewise APP/PS1/ASC^{-/-} animals showed substantially improved spatial memory performance (FIG. 3c-f, FIG. 13b). This protective effect of ASC deficiency remained detectable at 12 months of age (FIG. 13e-h).

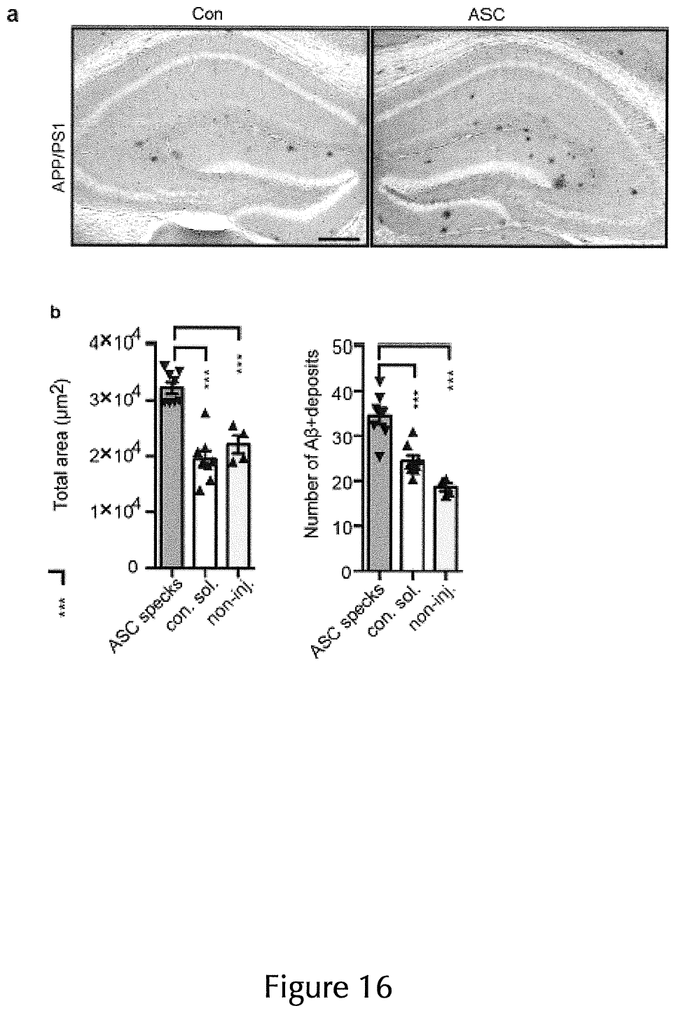

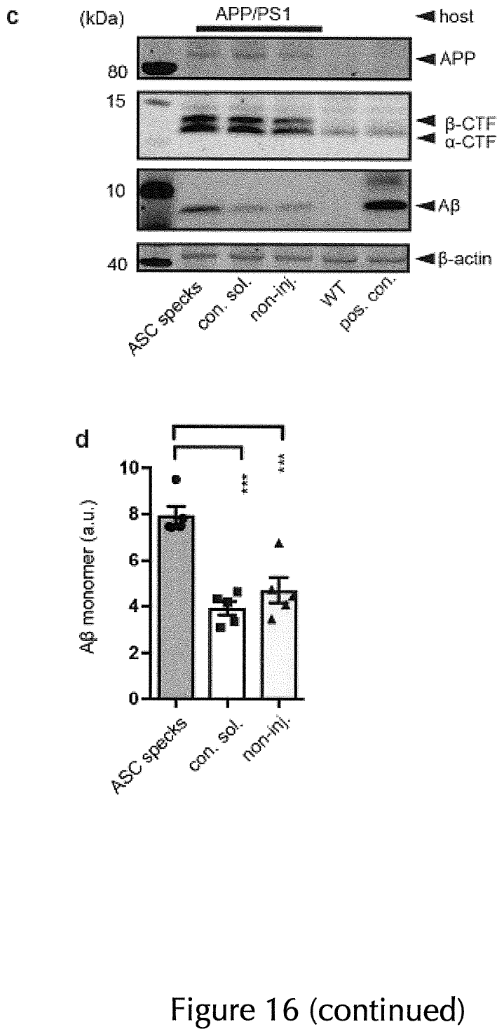

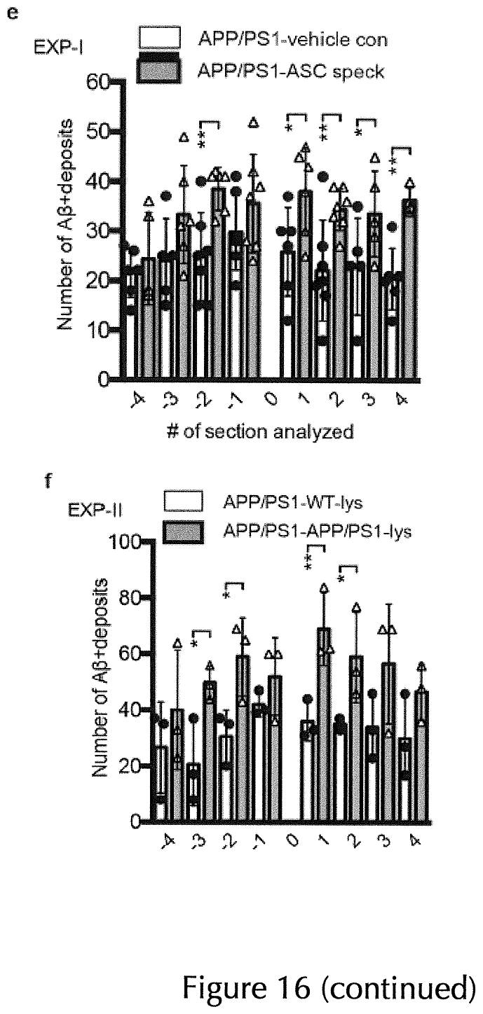

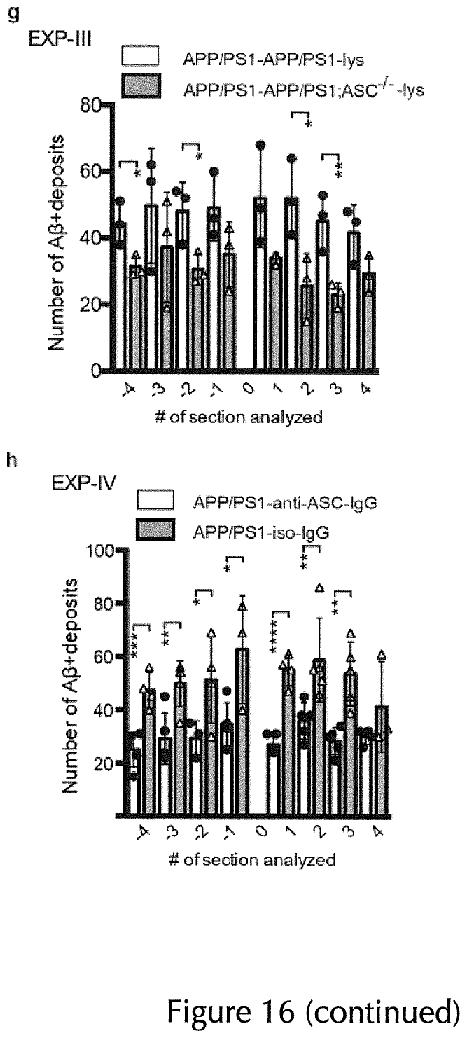

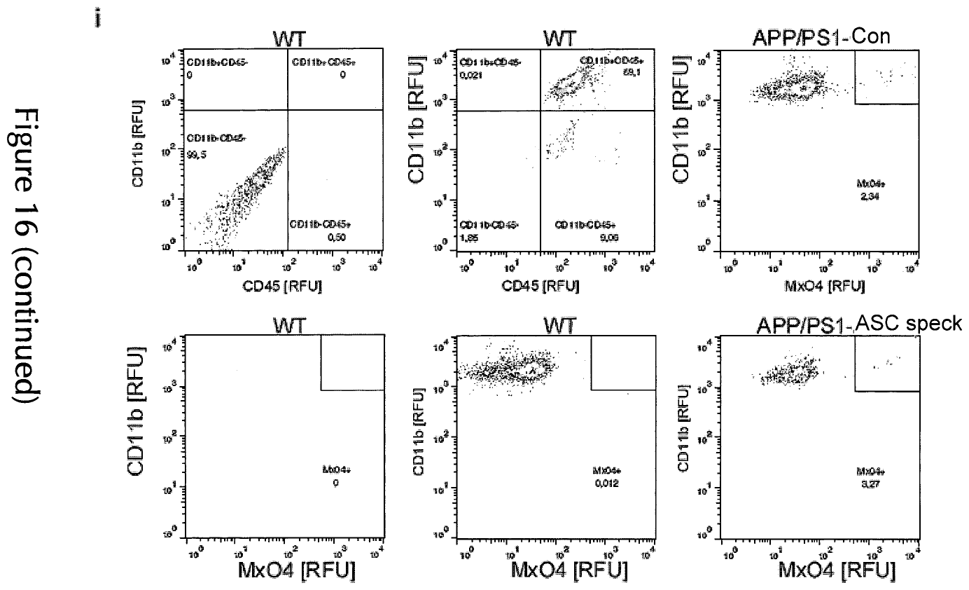

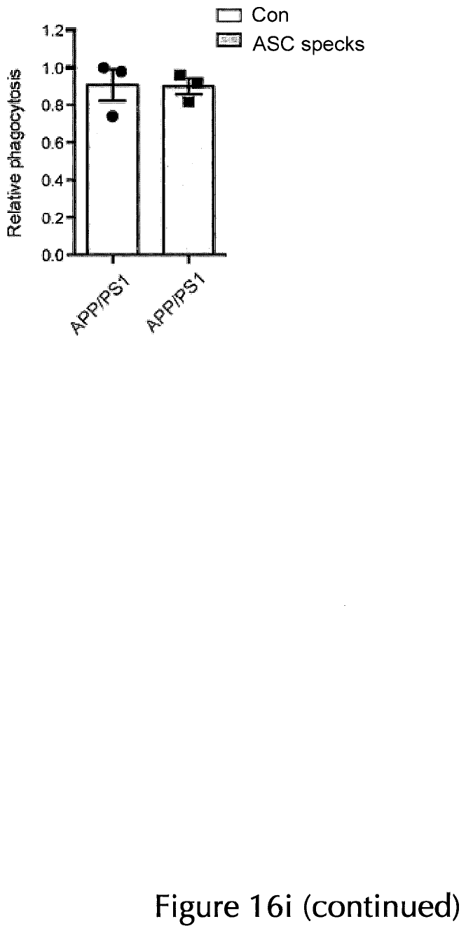

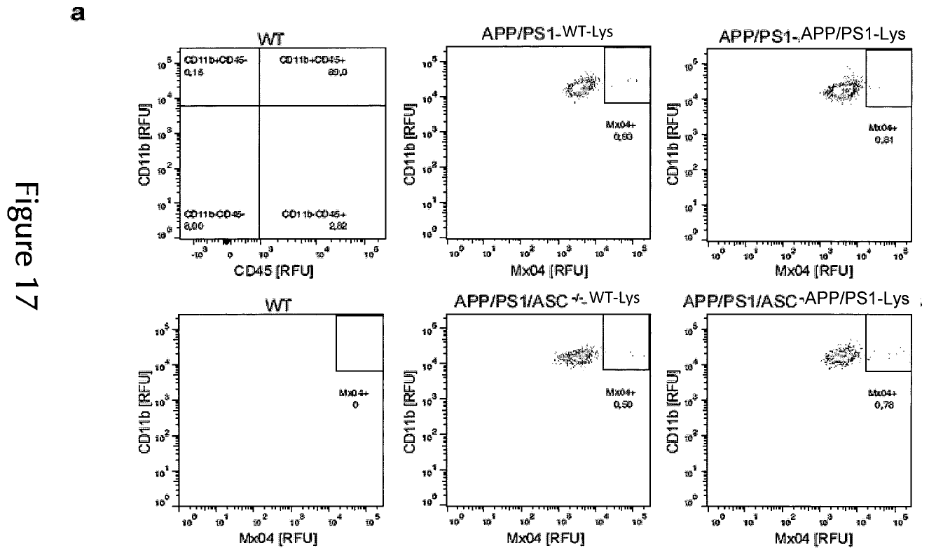

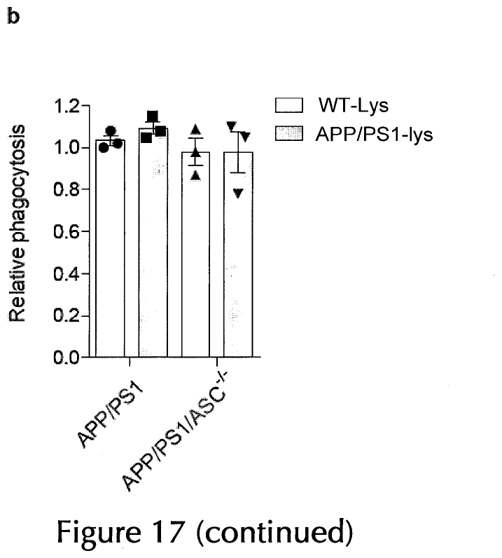

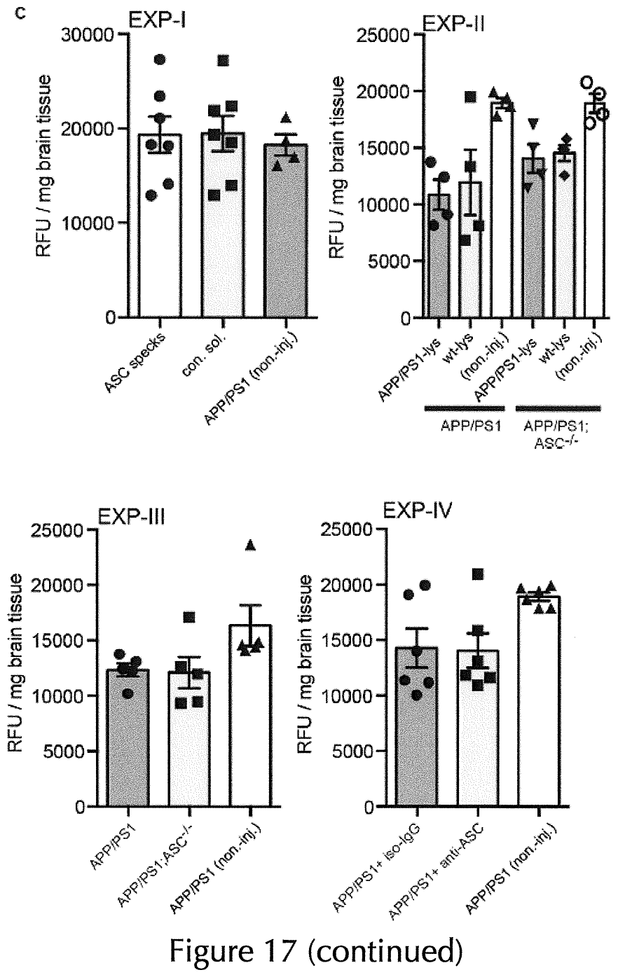

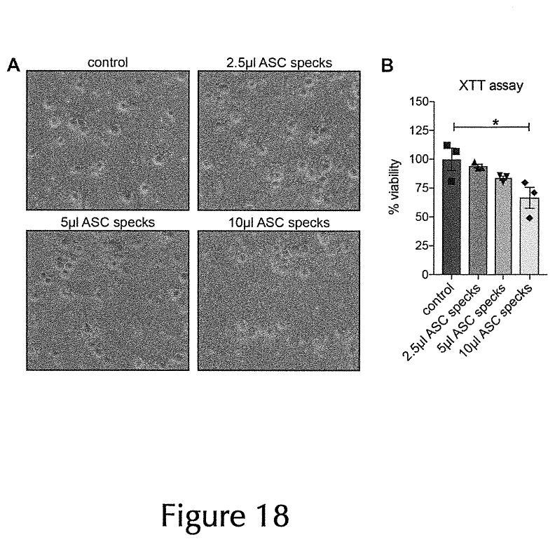

To investigate if ASC acts as an Aβ cross-seeding agent in vivo, we injected cell supernatant-derived or purified ASC specks into the hippocampus of 3-month old APP/PS1 mice and analyzed their brains at 6 months of age for Aβ deposition (FIG. 14b-f). Intrahippocampal ASC speck injection increased the number and total area of Aβ immunopositive deposits compared to the contralateral hippocampus receiving solvent control (FIG. 16a,b,e) without affecting phagocytosis (FIG. 16). This result was substantiated by immunoblot analysis of pooled brain homogenates generated from brain sections having a defined distance to the injection site, which showed a substantial increase of Aβ induced by ASC speck injection without changes in the APP expression or APP cleavage products (FIG. 16c,d). Previously, spreading of Aβ pathology was described in response to injection of APP transgenic animals with brain homogenates derived from APP or APP/PS1 transgenic animals^{9,23}. To test whether endogenous ASC contributes to this phenomenon, APP/PS1 or APP/PS1/ASC^{-/-} mice received intrahippocampal injections with an APP/PS1-derived brain homogenate, while the contralateral hippocampus was injected with a wild-type mouse brain homogenate. Animals were injected at 3 months and analyzed at 8 months of age (FIG. 15d). In APP/PS1 animals, the injection of APP/PS1 mouse brain-derived homogenates increased the number and total area of Aβ-positive deposits compared with the contralateral injection of wild-type mouse brain, confirming previous results (FIG. 3g,h)³. Importantly, this effect was completely absent in APP/PS1/ASC mice. Moreover, a comparison of the hemispheres of APP/PS1 and APP/PS1/ASC^{-/-} mice that had received APP/PS1 mouse brain homogenates revealed a strong difference in the number of Aβ deposits, their surface area, as well as their rostro-caudal spreading (FIG. 160. This immunohistochemical result was confirmed by ELISA for SDS soluble Aβ₁₋₄₀ and Aβ₁₋₄₂ or immunoblot analysis of brain homogenates and quantification of the Aβ monomer and oligomer fractions (FIG. 3i-k) without any changes in APP expression or cleavage products (FIG. 3j). We evaluated phagocytosis (FIG. 17 a,b), CASP1 activation or generation of Aβ degrading enzymes NEP and IDE (FIG. 14d). Results were equivalent for all parameters in the two genotypes, with the exception of IDE, which was increased in injected and non-injected ASC animals (FIG. 14d). Although this phenomenon was not paralleled by a significant increase of IDE activity (FIG. 17c) in the same brain tissue, we cannot exclude that an increase of IDE contributed to the overall effect. Nevertheless, all other in vivo experiments did not show significant differences of IDE levels or activity, but ASC speck-mediated modulation of Aβ pathology suggest the in vivo findings are, in large part, based on ASC-induced seeding. Together these experiments suggest that endogenous ASC represents a potential mechanism for induced Aβ spreading in this model.

### Example 4: ASC Speck Antibody Reduces Aβ Deposition

Next, the contribution of the endogenous ASC present in the injected brain homogenate was tested for its potential influence on Aβ spreading. In these experiments, 3-month old APP/PS1 mice received an intrahippocampal injection of mouse brain lysates either derived from APP/PS1 or APP/PS1/ASC^{-/-} animals (FIG. 15b,d) that were adjusted for equal amounts of Aβ (FIG. 15e). In line with the above findings, APP/PS1/ASC^{-/-} derived brain lysates showed a reduced capacity to increase the overall cerebral Aβ load and to induce rostro-caudal spreading of Aβ pathology when analyzed at 8 months of age (FIG. 4a-d, FIG. 16g). Thus, the combined evidence suggests that the ASC contained in the APP/PS1 brain homogenate is a contributing factor for the spreading of Aβ pathology. To verify a pathogenic role for ASC specks in vitro and in vivo, experiments targeting ASC specks by antibody co-incubation were performed. Employing ThT fluorescence spectroscopy, a specific anti-ASC-speck antibody was found to prevent ASC speck-induced aggregation of A1 in a concentration-dependent manner (FIG. 4e) without affecting Aβ aggregation perse (FIG. 4e). To further substantiate whether ASC specks were the mediating component responsible for the observed effect on Aβ spreading in vivo and to exclude the potential confounder of a difference in the gut microbiome in ASC-deficient mice²⁴, APP/PS1 animals received either an ASC speck-specific IgG or an isotype-specific IgG co-injected along with APP/PS1 brain homogenate (FIG. 4f-i). Targeting ASC specks by a single co-injection reduced rostro-caudal Aβ deposition (FIG. 16h). This effect was accompanied by a reduction of Aβ monomer and oligomers (FIG. 4g,i). Neither APP expression, APP cleavage products (FIG. 4c,i) nor IDE, NEP and CASP1 showed any changes (FIG. 14e,f) in the above described experiments.

Together these data suggest that ASC specks contribute to Aβ aggregation and spreading. Previous experiments reported that synthetic Aβ does not efficiently induce Aβ plaque formation, suggesting a need for a co-factor driving Aβ assembly and deposition. ASC specks released upon innate immune activation of microglia may represent such a cofactor, suggesting that inflammasome activation in the brain is connected to the progression of Aβ plaque formation in AD. Contrary to this putative mechanism, prion-related disease progression was unaffected by genetic deficiency for ASC or NLRP3 in a murine model of Scrapie²⁵, suggesting that mechanisms driving spreading differ between neurodegenerative disorders. The pathophysiological linkage of inflammasome responses with Aβ plaque spreading suggests that pharmacological targeting of inflammasomes could represent a novel treatment modality for AD.

Human anti-ASC speck antibodies could prevent cross-seeding of beta-amyloid peptides in the brain during aging. Aging associated immune senescence is characterized by compromised antibody generation and immune surveillance. Thus, immunesenescence may be associated with the reduced levels of autoantibodies directed against ASC specks. We propose to use endogenous anti ASC speck antibody titers as possible markers of disease progression, in particular during the clinically silent pre-stages of neurodegenerative disease such as Alzheimer's disease. Since beta-amyloid deposition also takes place in Lewy body dementia, the following mechanisms may in particular be used for the diagnosis and differential diagnosis of all forms of dementia.

Furthermore, ASC speck formation may occur as part of a well described innate immune reaction in other neurodegenerative disease such as Parkinson's disease, Multiple System Atrophy, Huntington's disease, Amyotrophic Lateral sclerosis and Sinocerebellar ataxias.

### Example 5: Primary Hippocampal Neurons Exposed to Increasing Concentrations of ASC Specks

Purification of ASC Specks

For the purification of ASC specks immortalized macrophages were used with an overexpression of NLRP3 and ASC. Two days following the seeding, the cells were harvested and pelleted. Cell lysis was carried out by a two-fold pellet volume CHAPS buffer (20 mM HEPES, 5 mM MgCl₂, 0.5 mM EGTA, 0.1% CHAPS, 0.1 mM PMSF, 1× protease inhibitor mix (Roche)) by a 25 fold filling by a 20 G needle on a syringe on ice. Upon centrifugation of the lysate for 8 minutes at 18.500×g the supernatants were collected and again centrifuged for 30 minutes at 100.000×g at 4° C. The resulting supernatants were taken away and filtered by using a 0.22 μm PVDF filter by centrifugation (5 minutes 18.500×g at 4° C.). Subsequently, ASC speck formation in the filtrates was induced by incubation for 30 minutes at 37° C. The ASC specks were centrifugated for 8 minutes at 600×g at 4° C. The pellet was dissolved in sterile PBS. The ASC speck containing solution was stored at 4° C.

Treatment of Primary Neurons by ASC Specks.

Hippocampal neurons were prepared from C57BU6N mice at E15-16. 70.000 cells per well were seeded at a 24 multiwell-plate. 12 days after seeding, the neurons were treated with various volumes of ASC speck solution for 24 hours. For measuring cell viability, the neurons were incubated—following the ASC speck treatment—by an XTT-solution (Cell Signalling) corresponding to the manufacturer's protocol. The read-out of the results was done after three hours on a plate read-out device (TECAN) at an absorption of 450 nm.

As confirmed by the inventors, the major inflammatory event observed e.g. in Alzheimer patients results from aggregation of beta-amyloid. That effect was found to be the primary cause for e.g. Morbus Alzheimer. Inflammation following AD aggregation is successfully overcome by using anti ASC speck ligands, in particular antibodies which counteract the effect of ASC specks contributing to A-beta-aggregation and spreading. It was thus the inventive finding (as shown by the in vivo-experiments of FIGS. 9F to 91) that an anti-ASC speck antibody effectively reduces ASC speck-based aggregation. The findings of Franklin et al. describe a distinct setting with an in vivo inflammasome activation triggered by the injection of silica crystals. Anti-ASC speck antibodies under such circumstances bind to ASC speck appearing after silica crystal triggered in vivo inflammasome activation.

### REFERENCES

1. Heneka, M. T., Kummer, M. P. & Latz, E. Innate immune activation in neurodegenerative disease. Nat. Rev. Immunol. 14, 463-477 (2014).
2. Lambert, J. C. et al. Meta-analysis of 74,046 individuals identifies 11 new susceptibility loci for Alzheimer's disease. Nat. Genet. 45, 1452-1458 (2013).
3. Yokoyama, J. S. et al. Association Between Genetic Traits for Immune-Mediated Diseases and Alzheimer Disease. JAMA Neurol (2016). doi:10.1001/jamaneurol.2016.0150
4. Gjoneska, E. et al. Conserved epigenomic signals in mice and humans reveal immune basis of Alzheimer's disease. Nature 518, 365-369 (2015).
5. Zhang, B. et al. Integrated Systems Approach Identifies Genetic Nodes and Networks in Late-Onset Alzheimer's Disease. Cell 153, 707-720 (2013).
6. Raj, T. et al. Polarization of the effects of autoimmune and neurodegenerative risk alleles in leukocytes. Science 344, 519-523 (2014).
7. Karch, C. M., Cruchaga, C. & Goate, A. M. Alzheimer's disease genetics: from the bench to the clinic. Neuron 83, 11-26 (2014).
8. Musiek, E. S. & Holtzman, D. M. Three dimensions of the amyloid hypothesis: time, space and ‘wingmen’. Nat. Neurosci. 18, 800-806 (2015).
9. Jucker, M. & Walker, L. C. Self-propagation of pathogenic protein aggregates in neurodegenerative diseases. Nature 501, 45-51 (2013).
10. Jack, C. R., Jr et al. Tracking pathophysiological processes in Alzheimer's disease: an updated hypothetical model of dynamic biomarkers. Lancet Neurol 12, 207-216 (2013).
11. Heneka, M. T., Golenbock, D. T. & Latz, E. Innate immunity in Alzheimer's disease. Nat. Immunol. 16, 229-236 (2015).
12. Heneka, M. T. et al. NLRP3 is activated in Alzheimer's disease and contributes to pathology in APP/PS1 mice. Nature 493, 674-678 (2013).
13. Lu, A. et al. Unified polymerization mechanism for the assembly of ASC-dependent inflammasomes. Cell 156, 1193-1206 (2014).
14. Masumoto, J. et al. ASC, a novel 22-kDa protein, aggregates during apoptosis of human promyelocytic leukemia HL-60 cells. J. Biol. Chem. 274, 33835-33838 (1999).
15. Cai, X. et al. Prion-like polymerization underlies signal transduction in antiviral immune defense and inflammasome activation. Cell 156, 1207-1222 (2014).
16. Franklin, B. S. et al. The adaptor ASC has extracellular and ‘prionoid’ activities that propagate inflammation. Nat. Immunol. 15, 727-737 (2014).
17. Baroja-Mazo, A. et al. The NLRP3 inflammasome is released as a particulate danger signal that amplifies the inflammatory response. Nat. Immunol. 15, 738-748 (2014).
18. Dick, M. S., Sborgi, L., Ruhl, S., Hiller, S. & Broz, P. ASC filament formation serves as a signal amplification mechanism for inflammasomes. Nat Commun 7, 11929 (2016).
19. Tomiyama, T. et al. A new amyloid beta variant favoring oligomerization in Alzheimer's-type dementia. Ann. Neurol. 63, 377-387 (2008).
20. Borchelt, D. R. et al. Familial Alzheimer's disease-linked presenilin 1 variants elevate Abeta1-42/1-40 ratio in vitro and in vivo. Neuron 17, 1005-1013 (1996).
21. Scheuner, D. et al. Secreted amyloid beta-protein similar to that in the senile plaques of Alzheimer's disease is increased in vivo by the presenilin 1 and 2 and APP mutations linked to familial Alzheimer's disease. Nat. Med. 2, 864-870 (1996).
22. Youm, Y.-H. et al. Canonical Nlrp3 inflammasome links systemic low-grade inflammation to functional decline in aging. Cell Metab. 18, 519-532 (2013).
23. Meyer-Luehmann, M. et al. Exogenous induction of cerebral beta-amyloidogenesis is governed by agent and host. Science 313, 1781-1784 (2006).
24. Elinav, E. et al. NLRP6 inflammasome regulates colonic microbial ecology and risk for colitis. Cell 145, 745-757 (2011).
25. Nuvolone, M., Sorce, S., Schwarz, P. & Aguzzi, A. Prion pathogenesis in the absence of NLRP3/ASC inflammasomes. PLoS ONE 10, e0117208 (2015).
26. Yamanaka, M. et al. PPARγ/RXRα-induced and CD36-mediated microglial amyloid-β phagocytosis results in cognitive improvement in amyloid precursor protein/presenilin 1 mice. J. Neurosci. 32, 17321-17331 (2012).
27. Fernandes-Alnemri, T. & Alnemri, E. S. Assembly, purification, and assay of the activity of the ASC pyroptosome. Meth. Enzymo. 442, 251-270 (2008).
28. Fernandes-Alnemri, T. et al. The pyroptosome: a supramolecular assembly of ASC dimers mediating inflammatory cell death via caspase-1 activation. Cell Death Differ. 14, 1590-1604(2007).
29. Wahle, T. et al. GGA1 is expressed in the human brain and affects the generation of amyloid beta-peptide. J. Neurosci. 26, 12838-12846 (2006).
30. Kumar, S. et al. Extracellular phosphorylation of the amyloid β-peptide promotes formation of toxic aggregates during the pathogenesis of Alzheimer's disease. EMBO J. 30, 2255-2265(2011).
31. Rostagno, A. & Ghiso, J. Isolation and biochemical characterization of amyloid plaques and paired helical filaments. Curr Protoc Cell Biol Chapter 3, Unit 3.33 3.33.1-33 (2009).
32. Fritschi, S. K. et al. As seeds resist inactivation by formaldehyde. Acta Neuropathol. 128, 477-484 (2014).
33. Jager, S. et al. alpha-secretase mediated conversion of the amyloid precursor protein derived membrane stub C99 to C83 limits Abeta generation. J. Neurochem. 111, 1369-1382 (2009).

1. A method of treatment or prevention of neurodegenerative diseases comprising: administering a ligand of apoptosis-associated speck-like protein containing a CARD (ASC).

2. The method according to claim 1, wherein said neurodegenerative diseases are associated with the formation of ASC aggregates and/or amyloid-β aggregates.

3. The method according to claim 1, wherein said neurodegenerative diseases are characterized and/or accompanied by dementia.

4. The method according to claim 1, wherein said neurodegenerative diseases are selected from Alzheimer's Disease, Parkinsons's Disease, Huntington's disease, Multiple System Atrophy, Amyotrophic Lateral Sclerosis, Sinocerebellar ataxia, Frontotemporal Dementia, Frontotemporal Lobar Degeneration, Mild Cognitive Impairment, Parkinson-plus syndromes, Pick disease, Progressive isolated aphasia, Grey-matter degeneration [Alpers], Subacute necrotizing encephalopathy, and Lewy body dementia.

5. The method according to claim 1, wherein said ligand modulates, preferably prevents, reduces, inhibits or blocks the biological functions and activities of ASC.

6. The method according to claim 5, wherein said biological functions and activities of ASC include its capability of forming aggregates and/or inducing or promoting the formation of amyloid-β aggregates.

7. The method according to claim 1, herein said ligand specifically interacts with, preferably binds to, ASC.

8. The method according to claim 1, wherein ASC comprises or consists of an amino acid sequence corresponding to SEQ ID NO: 1, or a homolog, isoform, variant or fragment thereof.

9. The method according to claim 1, wherein said ASC ligand specifically interacts with, preferably binds to, a PYD domain of ASC or an epitope located within said PYD domain, and/or to a CARD domain or an epitope located within said CARD domain.

10. The method according to claim 9, wherein said epitope located in the PYD domain comprises amino acids K21, K22 and/or K26 of an amino acid sequence corresponding to SEQ ID NO: 1.

11. The method according claim 1, wherein said ligand is selected from an antibody, a protein, a peptide, a nucleic acid, and a small molecule organic compound.

12. The method according to claim 11, wherein said ligand is a monoclonal or polyclonal antibody, or a variant, fragment or derivative thereof.

13. The method according to claim 12, wherein said antibody variant is selected from a chimeric antibody variant and a humanized antibody variant.

14. The method according to claim 12, wherein said derivative is selected from an scFv, a diabody, a linear antibody, a single-chain antibody, a bi- or multispecific antibody, an antibody-drug conjugate and a chimeric antigen receptor.

15. The method according to claim 11, wherein said antibody is selected from 653902 clone TMS-1 (BioLegend, San Diego, Calif., U.S.A.); AL177 (AdipoGen, AG-25B-0006-C100, Liestal, Switzerland), LS-C331318-50 (LifeSpan BioSciences); AF3805 (R&D Systems); NBP1-78977 (Novus Biologicals); 600-401-Y67 (Rockland Immunochemicals, Inc.); AF3805-SP (R&D Systems); orb160033 (Biorbyt); orb223237 (Biorbyt); 676502 (BioLegend); 653902 (BioLegend); MBS150936 (MyBioSource.com); MBS420732 (MyBioSource.com); MBS9401386 (MyBioSource.com); MBS9404874 (MyBioSource.com); MBS8504703 (MyBioSource.com); MBS841111 (MyBioSource.com); AB3607 (Merck); 04-147 clone 2EI-7 (Merck); NB300-1056 (Novus Biologicals); NB100-56075 (Novus Biologicals); NBP1-78978 (Novus Biologicals); NBP1-78977SS (Novus Biologicals); NBP1-78978SS (Novus Biologicals); NBP1-77297 (Novus Biologicals); AP07343PU-N (OriGene Technologies); AP06792PU-N (OriGene Technologies); AM26452AF-N (OriGene Technologies); AP32825PU-N (OriGene Technologies); AP23602PU-N (OriGene Technologies); TA306044 (OriGene Technologies); 3291-100 (BioVision); 3291-30T (BioVision); STJ25245 (St John's Laboratory); STJ91730 (St John's Laboratory); LS-C180180-100 (LifeSpan BioSciences); LS-C48292-100 (LifeSpan BioSciences); STJ70108 (St John's Laboratory); STJ113135 (St John's Laboratory); LS-C155196-100 (LifeSpan BioSciences); GTX22236 (GeneTex); GTX102474 (GeneTex); GTX28394 (GeneTex); D086-3 (MBL International); 13833S (Cell Signaling Technology); CAE04552 (Biomatik); ADI-905-173-100 (Enzo Life Sciences, Inc.); 40618 (Signalway Antibody LLC); E-AB-30582 (Elabscience Biotechnology Inc.); ab180799 (Abcam); 168-10230 (Raybiotech, Inc.); ER-03-0001 (Raybiotech, Inc.); A3598-05B-100ug (United States Biological); A3598-05N-50ug (United States Biological); AP5631 (ECM Biosciences); ABIN1001824 (antibodies-online); 2287 (ProSci, Inc); 70R-11744 (Fitzgerald Industries International); AHP1606 (Bio-Rad); PA1-41405 (Invitrogen Antibodies); PA5-19957 (Invitrogen Antibodies); PA5-27715 (Invitrogen Antibodies); PA1-9010 (Invitrogen Antibodies); 10500-1-AP (Proteintech Group Inc); sc-514414 (Santa Cruz Biotechnology, Inc.); and sc-514559 (Santa Cruz Biotechnology, Inc.), and a variant, fragment or derivative thereof.

16. The method according to claim 11, wherein said protein or said peptide is selected from a soluble receptor, an adnectin, an anticalin, a DARPin, an avimer, an affibody, a peptide aptamer and a variant, fragment or derivative thereof.

17. The method according to claim 11, wherein said nucleic acid is selected from an aptamer, an antisense nucleic acid, a miRNA, a siRNA and a shRNA.

18. A nucleic acid molecule encoding an ASC ligand according to claim 1.

19. A vector comprising the nucleic acid molecule according to claim 18.

20. A host cell comprising the nucleic acid molecule according to claim 18.

21. A pharmaceutical composition comprising at least one ASC ligand according to claim 1 and at least one pharmaceutically acceptable excipient.

22. (canceled)

23. The pharmaceutical composition according to claim 21, further comprising at least one additional active agent selected from nootropic agents, neuroprotectants, antiparkinsonian drugs, amyloid protein deposition inhibitors, beta amyloid synthesis inhibitors, antidepressants, anxiolytic drugs, antipsychotic drugs and anti-multiple sclerosis drugs.

24.-42. (canceled)
